(19) United States
(12) Patent Application Publication
(10) Pub. No.: US 2025/0066478 A1
(43) Pub. Date: Feb. 27, 2025

(54) HIGH-EFFICIENCY, CONDITIONALLY-ACTIVATED ANTIBODY DISCOVERY AND MASKED ANTIBODIES

(71) Applicant: iBio, Inc., Bryan, TX (US)

(72) Inventors: Matthew P. Greving, Rancho Santa Fe, CA (US); Alexander T. Taguchi, San Diego, CA (US); Cody Allen Moore, Del Mar, CA (US)

(21) Appl. No.: 18/813,515

(22) Filed: Aug. 23, 2024

Related U.S. Application Data
(60) Provisional application No. 63/578,767, filed on Aug. 25, 2023.

(51) Int. Cl. C07K 16/28; C07K 16/30; G16B 15/00

(52) U.S. Cl. CPC C07K 16/2809; C07K 16/3092; G16B 15/00; C07K 2317/565; C07K 2317/73

(57) ABSTRACT

Provided herein are methods for making, and epitope-targeted, conditionally-activated, pro-drug, antibody, comprising: complementarity determining regions (CDRs) from an antibody identified from an in vivo, in vitro, or in silico antibody library; one or more engineered epitope masks connected to a linker, wherein the linker comprises a peptide, a polymer, or a chemical-linker that is cleaveable in vivo at a target site by an enzyme or cleaved chemically, and wherein the one or more engineered epitope masks binds the epitope-specific antibody at the CDRs of the epitope-specific antibody; and wherein the one or more engineered epitope masks linked to the antibody via the linker, wherein the masks are conditionally bound to the CDRs of the epitope-specific antibody, and wherein cleavage of the linker releases the one or more engineered epitope masks from the antigen binding site.

### CROSS-REFERENCE TO RELATED APPLICATIONS

This application claims priority to U.S. Provisional Application Ser. No. 63/578,767, filed Aug. 25, 2023, the entire contents of which are incorporated herein by reference.

### STATEMENT OF FEDERALLY FUNDED RESEARCH

Not applicable.

### INCORPORATION-BY-REFERENCE OF MATERIALS FILED ON COMPACT DISC

The application contains a Sequence Listing which has been submitted electronically in .XML format and is hereby incorporated by reference in its entirety. Said .XML copy, created on Oct. 10, 2024, is named “IBIO1042.xml” and is 579,916 bytes in size. The sequence listing contained in this .XML file is part of the specification and is hereby incorporated by reference herein in its entirety.

### BACKGROUND

Without limiting the scope of the disclosure, its background is described in connection with masked antibodies.

One such antibody is a probody in which the antibody and masking peptide are two separate discovery cycles, and the masking peptide discovered using random peptide library phage display. An example of this antibody is made by Cytomx.

Another conditional active biologic is an antibody discovered under the condition that affinity is significantly stronger in the acidic conditions of the tumor microenvironment. An example of this conditional active biologic is made by BioAtla.

Another example is hemibody that is (inactive) when there is a variable heavy chain (VH) and a variable light chain (VL) pairing that is inhibited in normal conditions, but becomes active when the VH:VL pairs to a functional antibody under disease conditions. One example is the hemibody in which the antibody binds to two disease-specific antigens concurrently. The other is known as PrecisionGATE in which a blocking moiety is cleaved by, e.g., a tumor protease.

Yet another example is the coiled-coil mask, in which the coiled-coil domain inhibits antigen binding via steric hindrance, and the antibody is activated upon proteolytic cleavage of the coiled-coil domain. An example of this antibody is made by Seagen.

Another example is known as Protect, in which, PD-L1:PD-1 heterodimer inhibits antigen binding via steric hindrance and the antibody is activated upon proteolytic cleavage of the PD-L1 domain. The PD-1 domain increases binding avidity for PD-L1 positive tumors along with tumor antigen (TAA) binding arm. An example of this antibody is made by Zymeworks.

Another example is an anti-idiotypic conditionally activate antibody. In this example, an anti-idiotype anti-CD3 mask blocks CD3 T cell engager arm and the antibody is activated upon proteolytic cleavage of the anti-idiotype anti-CD3 mask. An example of this antibody is made by Roche.

Yet another example is known as XTEN, in which a disordered polymer mask inhibits antigen binding via steric hindrance. The antibody is activated upon proteolytic cleavage of the polymer mask. An example of this antibody is made by Amunix/Sanofi.

Another example is known as Cobra. In this system, inactive VL and VH domains pair with anti-CD3 VL and VH domains to block active anti-CD3 antibody VH:VL pairing. The antibody is activated upon proteolytic cleavage of the inactive VL and VH domains, enabling active anti-CD3 antibody VH:VL pairing. An example of this antibody is made by Maverick/Takeda.

Another example is known as ProTriTAC. In this construct an anti-albumin plus peptide masks the anti-CD3 domain in the inactive prodrug. The antibody is proteolytically cleavaged and the anti-albumin plus peptide mask activates the anti-CD3 domain. An example of this antibody is made by Harpoon.

Finally, another example is known as ATP Switch. In this example, the antibody is discovered under the condition that affinity is significantly stronger in the presence of ATP. When ATP is high in the tumor-microenvironment, the antibody is activated. An example of this antibody is made by Chugai.

Despite these advances, a need remains for an efficient method to produce highly-specific, prodrug antibody compositions that are conditionally activated under specific conditions at a target site, but remain blocked in non-target tissue.

### SUMMARY

As embodied and broadly described herein, an aspect of the present disclosure relates to a method of making an epitope-targeted, conditionally-activated, pro-drug, antibody in a single discovery cycle comprising: obtaining an epitope-specific antibody comprising an antigen binding site with complementarity determining regions (CDRs) from an in vivo, in vitro, or in silico antibody library; concurrently designing or obtaining in silico one or more engineered epitope masks connected to linker, wherein the linker comprises a peptide, a polymer, or a chemical-linker that is cleaved in vivo at a target site by an enzyme or cleaved chemically, or a structural-switch linker that switches to an active state at a target site by one or more environmental condition(s), and wherein the one or more engineered epitope masks binds the epitope-specific antibody at the CDRs of the epitope-specific antibody; and making the epitope-targeted, conditionally-activated, pro-drug, antibody as a fusion protein or an antibody with the one or more engineered epitope masks linked to the antibody via the linker, wherein the one or more engineered epitope masks are conditionally bound to the CDRs of the epitope-specific antibody, wherein cleavage of the linker releases the one or more engineered epitope masks from the antigen binding site. In one aspect, the peptide, polymer, or chemical-linker epitope are not obtained by steric inhibition in vitro. In another aspect, the antibody and the peptide, polymer, or chemical-linker epitope are selected concurrently in a single discovery cycle. In another aspect, the linker is a cleavable peptide, polymer, or chemical linker. In another aspect, the linker is a structure-switch peptide, polymer, or chemical linker. In another aspect, the method further comprises concurrently designing in silico the peptide, polymer, or epitope and the cleavable or structure-switch peptide, polymer, or chemical link. In another aspect, the method further comprises designing a cleavable or structure-switch peptide, polymer, or chemical linker to at least one of: increase or decrease proteolytic or cleavage activity of the antibody, one or more engineered-epitopes, the linker, or all three; increase or decrease an on/off tissue conditional-activation of the antibody, one or more engineered-epitopes, the linker, or all three; increase or decrease solubility of the antibody, one or more engineered-epitopes, the linker, or all three; increase or decrease expression of the antibody; increase or decrease stability of the antibody, one or more engineered-epitopes, the linker, or all three; increase or decrease immunogenicity of the antibody, one or more engineered-epitopes, the linker, or all three; mask the complementarity determining regions (CDRs) that generate anti-drug antibodies; add an inhibitory or stimulatory activity in the one or more engineered-epitopes, the linker, or both; or add a cell or tissue localization in the one or more engineered-epitopes, the linker, or both. In another aspect, the peptide, polymer, or epitope and the linker are not designed concurrently. In another aspect, the method further comprises modifying the peptide, polymer, or chemical linker of the linker to increase or decrease cleavage of the linker at the target site. In another aspect, the one or more engineered epitope masks and the linker are not obtained from a random library of peptides, polymers, or chemical linkers. In another aspect, the one or more engineered epitope masks and the linker are modeled in silico to fit an antigen binding site of a specific antibody. In another aspect, the method further comprises obtaining the epitope-specific antibody comprising and the one or more engineered epitope masks and the linker further comprises: training a machine learning model based on an epitope-specific antibody record and one or more engineered epitopes, or representations thereof, and a first plurality of scores, each epitope-specific antibody record from the first plurality of epitope-specific antibody record and one or more engineered epitopes associated with each score from the first plurality of scores; and executing, after the training, the machine learning model to generate a second plurality of epitope-specific antibody records and one or more engineered epitopes having at least one desired score; the second plurality of epitope-specific antibody records configured to be received as input in computational protein modeling to generate the one or more engineered epitopes based on a second plurality of epitope-specific antibody record with the one or more engineered epitopes in an antigen binding site of the epitope-specific antibody. In another aspect, the method further comprises receiving a representation of the one or more engineered epitope masks and the linker; and generating the first plurality of epitope-specific antibody records from a predetermined portion of the one or more engineered epitope masks and the linker, each epitope-specific antibody records from the first plurality of epitope-specific antibody records comprising target residue positions and linker residue positions, each target residue position corresponding to one target residue from a plurality of target residues of the one or more engineered epitope masks and the linker. In another aspect, the method further comprises labeling a first plurality of epitope-specific antibody records by, for each epitope-specific antibody records from the first plurality of epitope-specific antibody records: performing computational protein modeling on that epitope-specific antibody record to generate a polypeptide structure, calculating a score for the polypeptide structure, and associating the score with that epitope-specific antibody record.

As embodied and broadly described herein, an aspect of the present disclosure relates to an epitope-targeted, conditionally-activated, pro-drug, antibody comprising: an antibody binding site with complementarity determining regions (CDRs) from an epitope-specific antibody identified from an in vivo, in vitro, or in silico antibody library; and one or more engineered epitope masks connected to a linker, wherein the linker comprises a peptide, a polymer, or a chemical-linker that is cleavable in vivo at a target site by an enzyme or cleaved chemically, and wherein the one or more engineered epitope masks binds the epitope-specific antibody at the CDRs of the epitope-specific antibody; and wherein the epitope-targeted, conditionally-activated, pro-drug, antibody is a fusion protein or an antibody with the one or more engineered epitope masks linked to the antibody via the linker, wherein the one or more engineered epitope masks are conditionally bound to the CDRs of the epitope-specific antibody, and wherein cleavage of the linker releases the one or more engineered epitope masks from the antigen binding site from the epitope-targeted, conditionally-activated, pro-drug, antibody. In one aspect, the antibody comprises: a heavy chain variable domain (VH) complementarity determining region (CDRs) 1 to CDR3 comprising an amino acid sequence of any one of the following SEQ ID NOS: 1, 2, 3; 15, 16, 17; 29, 30, 31; 32, 33, 34; 35, 36, 37; 38, 39, 40; 41, 42, 43; 44, 45, 46; 47, 48, 49; 50, 51, 52; 53, 54, 55; 56, 57, 58; 59, 60, 61; 62, 63, 64; 65, 66, 67; 68, 69, 70; 71, 72, 73; 74, 75, 76; 77, 78, 79; 80, 81, 82; 83, 84, 85; 86, 87, 88; 89, 90, 91; 92, 93, 94; 95, 96, 97; 98, 99, 100; 101, 102, 103; 104, 105, 106; 107, 108, 109; 110, 111, 112; 113, 114, 115; 116, 117, 118; 119, 120, 121; 122, 123, 124; 125, 126, 127; 128, 129, 130; 131, 132, 133; 134, 135, 136; 137, 138, 139; 140, 141, 142; 143, 144, 145; 146, 147, 148; 149, 150, 151; 152, 153, 154; 155, 156, 157; 158, 159, 160; 161, 162, 163; 164, 165, 166; 167, 168, 169; 170, 171, 172; 173, 174, 175; 176, 177, 178; 179, 180, 181; 182, 183, 184; 185, 186, 187; 188, 189, 190; 191, 192, 193; 194, 195, 196; 197, 198, 199; 200, 201, 202; 203, 204, 205; 206, 207, 208; 209, 210, 211; 212, 213, 214; 215, 216, 217; 218, 219, 220; 221, 222, 223; 224, 225, 226; 227, 228, 229; 230, 231, 232; 233, 234, 235; 236, 237, 238; 239, 240, 241; 242, 243, 244; 245, 246, 247; 248, 249, 250; 251, 252, 253; 254, 255, 256; 257, 258, 259; 260, 261, 262; 263, 264, 265; 266, 267, 268; 269, 270, 271; 272, 273, 274; 275, 276, 277; 278, 279, 280; or 281, 282, 283; a light chain variable domain (VL) CDR1 to CDR3 comprising the amino acid sequence of any one of the following SEQ ID NOs: 4, 5, 6; 18, 19, 20; 284, 285, 286; 287, 288, 289; 290, 291, 292; 293, 294, 295; 296, 297, 298; 299, 300, 301; 302, 303, 304; 305, 306, 307; 308, 309, 310; 311, 312, 313; 314, 315, 316; 317, 318, 319; 320, 321, 322; 323, 324, 325; 326, 327, 328; 329, 330, 331; 332, 333, 334; 335, 336, 337; 338, 339, 340; 341, 342, 343; 344, 345, 346; 347, 348, 349; 350, 351, 352; 353, 354, 355; 356, 357, 358; 359, 360, 361; 362, 363, 364; 365, 366, 367; 368, 369, 370; 371, 372, 373; 374, 375, 376; 377, 378, 379; 380, 381, 382; 383, 384, 385; 386, 387, 388; 389, 390, 391; 392, 393, 394; 395, 396, 397; 398, 399, 400; 401, 402, 403; 404, 405, 406; 407, 408, 409; 410, 411, 412; 413, 414, 415; 416, 417, 418; 419, 420, 421; 422, 423, 424; 425, 426, 427; 428, 429, 430; 431, 432, 433; 434, 435, 436; 437, 438, 439; 440, 441, 442; 443, 444, 445; 446, 447, 448; 449, 450, 451; 452, 453, 454; 455, 456, 457; 458, 459, 460; 461, 462, 463; 464, 465, 466; 467, 468, 469; 470, 471, 472; 473, 474, 475; 476, 477, 478; 479, 480, 481; 482, 483, 484; 485, 486, 487; 488, 489, 490; 491, 492, 493; 494, 495, 496; 497, 498, 499; 500, 501, 502; 503, 504, 505; 506, 507, 508; 509, 510, 511; 512, 513, 514; 515, 516, 517; 518, 519, 520; 521, 522, 523; 524, 525, 526; 527, 528, 529; 530, 531, 532; 533, 534, 535; or 536, 537, 538. In another aspect, the antibody comprises a VH and VL pair comprising the amino acid sequence of any one of the following SEQ ID NOS: 7 and 8, 9 and 10, 21 and 22, or 23 and 24. In another aspect, the antibody is a monoclonal antibody. In another aspect, the antibody is a full-length antibody. In another aspect, the antibody is an antibody fragment. In another aspect, the antibody is fused to an Fc domain of any one of the following: human IgG1, human IgG2, human IgG3, and human IgG4. In another aspect, the antibody specifically binds to: 4-1BBL, AFP, AKAP-4, ALK, androgen receptor, bcr-abl, Clorf186, CA-125, CA6, CA6, CA19-9, CAMPATH-1, Carbonic anhydrase IX, carcinoembryonic antigen (CEA), CCR8, CD1, CD2, CD3, CD4, CD5, CD8, CD16A, CD19, CD1A, CD20, CD204, CD204, CD204, CD206, CD206, CD206, CD25, CD276, CD28, CD30, CD301, CD301, CD32A, CD32B, CD33, CD36, CD37, CD39, CD40, CD45, CD47, CD5, CD64, CD73, CEA, CLEC4C, CLDN16, CLDN6, CLDN18.2, CLEC10A, CLEC12A, CLECSA, CLEC9A, CMET, CTCFL, cyclin B1, CYP1B1, DC-SIGN, DEC-205, Dectin1, Dectin2, DLL3, EGFR, EGFRvIII, Endoglin, endosialin, EPCAM, EphA2, epidermal growth factor, ERG, ETV6-AML, ferritin, fibroblast activation protein (FAP), FLT3, folate-binding protein, Fos-related antigen 1, FRA, fucosyl GM1, G250, GD2, GD3, Glycoprotein A33, GloboH, GLP-3, GM2, GM3, gp100, HER2, HER2/neu, HER3, HLD-DR, HMWMAA, HPV E6, HPV E7, hTERT, HVEM, IL-2 receptor, IV1, Latent-TGFB, LCK, Legumain, Ley, LIV1, LMP2, LY6E, MAD-CT-1, MAD-CT-2, MAGE A1, MAGE A3, mannose scavenger receptor1, MARCO, MelanA/MART1, mesothelin (MSLN), metalloproteinase, ML-IAP, MSLN, MUC1, MUC15, MUC16, MYCN, NA17, NAPI2B, NAPI2B, NY-BR-1, NY-ESO-1, OX40L, OY-TES1, p185HER2, P53 mutant, P53 nonmutant, PAGE4, PAP, PAX3, PAX5, PDGFR-B, PDL, PDL1, PLAV1, polysialic acid, PR1, PSA, PSCA, PSMA, PTK7, Ras mutant, RGS5, RhoC, RON, ROR1, ROR2, RRC15, SART3, SIRPA, Sperm protein 17, SSX2, STn, survivin, TAG-72, tenascin, TGFB, Tie 3, TMEM238, TMPRSS3, TMPRSS4, Tn, TRA6, TROP2, TRP-2, tyrosinase, UPK1B, vascular endothelial growth factor, VEGFR, VEGFR2, VISTA, VTCN1, WT1, XAGE 1, or Y6E. In another aspect, the antibody is ABT806 (EGFRvIII), adecatumumab (EPCAM), alemtuzumab (CD33), AMG595 (EGFRvIII), anetumab (MSLN), anti-ACE2, anti-EphA2 (EphA2), anti-hyaluronidase, anti-neuraminidase, anti-NY-ESO-1, anti-PTK7 (PTK7), cetuximab (EGFR), cirmtuzumab (ROR1), clivatuzumab (MUC1), CT-011 (PD1), DS-8895a variant 1 (EphA2), DS-8895a variant 2 (EphA2), durvalumab (PDL1) anti-MAGE-A3, edrecolomab (EPCAM), farletuzumab (FRA/folate receptor alpha), Gemtuzumab ozogamicin (CD33), huDS6 (CA6), humanized Ab 2-3 (CEA), humanized PR1A3 (CEA), ibritumomab tiuxetan (CD52), IMAB362/claudiximab (Claudin18.2), ipilimumab (CTLA4), J591 variant 1 (PSMA), J591 variant 2 (PSMA), ladiratuzumab (LIV1), lifastuzumab (NAPI2B), MEDI-547 (EphA2), mirvetuximab (FRA), narnatumab (RON), nimotuzumab (EGFR), nivolumab (PD1), onartuzumab (c-MET), panitumumab (EGFR), patritumab (HER3), pembrolizumab (PD1), pertuzumab (HER2/neu), PF-06647020 (PTK7), RG7841 (LY6E), rituximab (CD20), rovalpituzumab (DLL3), sacituzumab (TROP2), sibrotuzumab (FAP), sofituzumab (MUC16), tositumomab (CD20), trastuzumab (HER2/neu), tremelimumab (CP-675,206)(CTLA4), or zalutumumab (EGFR). In another aspect, the linker is a peptide cleaved by actinidain, activated protein C, ADAM10, ADAM12, ADAMS, bromelain, bromelain, calpain, Caspase, caspase-3, Cathepsin, Cathepsin B, Cathepsin C, Cathepsin D, Cathepsin E, cathepsin G, Cathepsin K, Cathepsin L, Chymase, chymosin, chymotrypsin-like protease, CMV protease, collagenase, DESC1, dipeptidyl peptidase, dipeptidyl peptidase IV (DPPIV/CD26), disintegrin and metalloproteinase (ADAM), DPP-4, Elastase, elastase-like protease, enterokinase, Factor Xa, FAP, FAP (FAP-α), Granzyme B, Guanidinobenzoatase, Hepsin, HIV-1 protease, hK1, hK15, hK3, HSV protease, HtrAl, HumNeutrophil Elastase, interleukin-1β converting enzyme, kallikrein, kallikrein-related peptidase (KLK), Lactoferrin, legumain, Marapsin, mast cell chymase, mast cell tryptase, matriptase, Matriptase-2, matrix metalloprotease (MMP), metalloendopeptidase, metalloexopeptidase, Mirl-CP, metalloproteases (MMP1, MMP2, MMP3, MMP7, MMP8, MMP9, MMP10, MMP11, MMP12, MMP13, MMP14), nepenthesin, neutrophil elastase, neutrophil serine protease 4, NS3/4A, PACE4, papain, pepsin, plasmepsin, plasmin, prostate-specific antigen (PSA), proteinase 3, renin, secretase, stromelysin, subtilisin-like protease, thrombin, tissue plasminogen activator (tPA), transmembrane Serine Protease (TMPRSS), trypsin, trypsin-like protease, tryptase, type II transmembrane serine protease (TTSP), Type IV collagenase, urokinase plasminogen activator (uPA), or urokinase plasminogen activator receptor (uPAR). In another aspect, the linker comprises non-amino acid cleavage polymers or linkers selected from nucleic acids, lipids, carbohydrates, or chemical linkers, such as those that are cleaved or dissociated by radiation, electromagnetic, pH, chemically, or enzymatically. In another aspect, wherein the linker comprises a structure-switch linker that includes amino acid or non-amino acid polymers that adopt an inactivated prodrug conformation with the mask blocking the antibody-antigen binding site and upon exposure to specific microenvironmental conditions the linker switches to the activated antibody conformation. In another aspect, wherein the microenvironmental conditions are selected from pH, enzyme(s), or metabolite(s).

As embodied and broadly described herein, an aspect of the present disclosure relates to a method of treating a disease in a subject in need thereof, comprising administering to the subject a therapeutically effective amount of the epitope-targeted, conditionally-activated, pro-drug, antibody described hereinabove. In one aspect, the disease is an autoimmune disease, an inflammatory disease, an infectious disease, or a cancer. In another aspect, the cancer is selected from: acute lymphoblastic leukemia, acute myelogenous leukemia, adrenal glands cancer, bladder cancer, bone marrow cancer, breast cancer, chronic lymphocytic leukemia, chronic myeloid leukemia, central nervous system cancer, colorectal cancer, colorectal carcinoma, endometrial cancer, endometrial carcinoma, gastric cancer, gut carcinoma, head and neck squamous cell carcinoma, hematopoietic malignancies, liver cancer, lung carcinoma, lymph node cancer, melanoma, metastatic colorectal cancer, ovarian cancer, pancreatic adenocarcinoma, pituitary tumor, prostate cancer, prostate carcinoma, renal cell carcinoma, retinal cancer, sarcoma, skin cancer, spleen cancer, stomach cancer, thymus cancer, or thyroid cancer. In another aspect, the subject is human.

As embodied and broadly described herein, an aspect of the present disclosure relates to a nucleic acid encoding an epitope-targeted, conditionally-activated, pro-drug, antibody comprising: a nucleic acid encoding an antigen binding site with complementarity determining regions (CDRs) from an antibody identified from an in vivo, in vitro, or in silico antibody library; and one or more engineered epitope masks connected to a linker, wherein the linker comprises a peptide, a polymer, or a chemical-linker that is cleavable in vivo at a target site by an enzyme or cleaved chemically, and wherein the one or more engineered epitope masks binds the epitope-specific antibody at the CDRs of the epitope-specific antibody; and wherein the epitope-targeted, conditionally-activated, pro-drug, antibody is a fusion protein or an antibody with the one or more engineered epitope masks linked to the antibody via the linker, wherein the one or more engineered epitope masks are conditionally bound to the CDRs of the epitope-specific antibody, and wherein cleavage of the linker releases the one or more engineered epitope masks from the antigen binding site. In one aspect, the nucleic acid comprises: a first polynucleotide encoding a heavy chain variable domain having at least 95, 96, 97, 98, 99, or 100% sequence identity to SEQ ID NOS: 11, 13, 25, 27; and second polynucleotide encoding a light chain variable domain encoding polynucleotide having at least 95, 96, 97, 98, 99, or 100% sequence identity to SEQ ID NOS: 12, 14, 26, 28. In another aspect, the antibody is a monoclonal, bispecific, multivalent, multi-specific, diabody, chimeric, scFv antibody, or fragments thereof. In another aspect, an antibody binding domain is fused to an Fc domain of any one of the following: human IgG1, human IgG2, human IgG3, and human IgG4. In another aspect, the nucleic acid sequence is optimized for expression in a bacterial, fungal, mammalian, insect, or plant cell.

As embodied and broadly described herein, an aspect of the present disclosure relates to a vector comprising the nucleic acid described hereinabove. As embodied and broadly described herein, an aspect of the present disclosure relates to a host cell comprising nucleic acid the vector described hereinabove.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present application can be understood by reference to the following description taking in conjunction with the accompanying figures.

FIGS. 1A and 1B illustrate the dual approaches used to generate a diverse panel of anti-CD3 antibodies. FIG. 1A shows the use of the AI discovery engine applied to either a structure-epitope immunization and screen, or the use of a StableHu optimizer. FIG. 1B shows that the results of the structure-epitope immunization and screen, or the use of a StableHu optimizer are then screened for binding or T cell activation.

FIG. 2 shows that the engineered epitopes guide immunization to TCR-accessible CD3 epitopes and are potential masks for conditionally-activated antibodies.

FIG. 3A is a graph showing that engineered epitope 1 is a conditionally-activated mask for anti-CD3 clone UCHT1, measuring engineered epitope 1 binding to UCHT1.

FIG. 3B is a graph showing that engineered epitope 1 is a conditionally-activated mask for anti-CD3 clone UCHT1 which shows the conditionally-activated UCHT1 Ab binding to T cells.

FIG. 4 shows that engineered epitope 2 is a conditionally-activated mask for anti-CD3 clones SP34 and 6-F05.

FIG. 5 shows the method of immunizations steered to a MUC16 epitope that avoids epitope shedding.

FIG. 6 summarizes MUC16 engineered epitope is a cleavable mask for the 21G6 Anti-MUC16 antibody.

FIGS. 7A and 7B show that the conditionally-activated 21G6 Anti-MUC16 Clone Masked with engineered epitope only binds MUC16 membrane-proximal region after protease treatment. FIG. 7A shows the results from the construct with the long linker mask on VH domain. FIG. 7B shows the results from the construct with the long linker mask on VL domain.

FIG. 8 shows the results from a CD3 epitope masking construct by ELISA. CD3 ELISA: Coat plate with antibody, detect with RP-labeled CD3. A greater than 100-fold shift observed when comparing the masked CD3 antibody before (hexagon) and after (square) digestion with MMP9.

FIG. 9 shows T cell killing with masked CD3 bispecific. PBMC coculture with MDA-MB-231 breast cancer cell line. Antibodies added undigested, MMP9/other proteases produced by cancer cells. Bispecific with CD3 arm masked and cleavable linker had >>100-fold greater cell killing potency than the same molecule with an uncleavable linker, and was within 10-fold potency of the unmasked variant of the bispecific.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

As used herein, the term “antibody” is used in the broadest sense and includes a monoclonal antibody, polyclonal antibody, human antibody, humanized antibody, non-human antibody, chimeric antibody, a monovalent antibody, an antibody fragment, and a tandem scFv-Fc antibody. Antibody fragments of the disclosure retain antigen-binding specificity. Antibody fragments include antigen-binding fragments (Fab), variable fragments (Fv) containing VH and VL sequences, single chain variable fragments (scFv) containing VH and VL sequences linked together in one chain, single chain antibody fragments (scAb) or other antibody variable region fragments, such as retaining antigen binding specificity.

As used herein, the term “epitope-targeted, conditionally-activated, pro-drug, antibody” refers to an antibody having a cognate target (epitope targeted) to which the antigen-binding site binds specifically, however, that binding is conditioned on the removal of a mask that was designed in the form of an engineered epitope to specifically block the antigen-binding site of the antibody. The engineered epitope is connected to the antibody via a linker that is cleavable, wherein cleavage of the linker releases the engineered epitope, thereby unmasking the antigen-binding site and thus “activating” the antibody. Prior to activation, the antibody is a “pro-drug”, that is, it is not effective to bind its cognate target.

As used herein, a “target region” refers to a location in vivo or ex vivo in which a linker of the epitope-targeted, conditionally-activated, pro-drug, antibody is cleaved to release the one or more engineered epitope masks from the antigen binding site of the antibody binding domain of the antibody or antigen-binding fragment thereof, thereby activating the antibody.

As used herein, an “engineered epitope mask” refers to a peptide, polymer, or compound that is bound specifically to the antibody binding site of the antibody or antigen-binding fragment thereof, thereby blocking the antibody from binding to its cognate target.

As used herein, a “linker” refers to a peptide, polymer, or chemical linker that connects the engineered epitope mask with the antibody or antigen-binding fragment thereof. The linker may provide structure to the engineered epitope mask. The linker provides for the conditional release of the engineered epitope mask from the antibody or antigen-binding fragment thereof, such as exposure to an enzyme (e.g., peptidase, nuclease, etc.) or conditions at the target sites (e.g., pH, temperature, chemical environment, presence of an activation signal or physical effect on the linker, such as light, electromagnetic, ultrasound, etc.). Cleavage of the linker leads to the release of the engineered epitope mask from the antibody or antigen-binding fragment thereof, thereby making available the activating the antigen-binding domain of the antibody or binding fragment thereof.

The linker can also include a peptide flexible linker, which is often comprised of helix- and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their serum half-life by using modified Fc regions or mutations to the various constant regions, as are known in the art.

As used herein, the term “masked” refers to having an engineered epitope that blocks the binding site of an antigen-binding portion of the epitope-targeted, conditionally-activated, pro-drug, antibody. The “mask” can be removed from the antigen-binding portion of an antibody thereby “activating” the ability of the antigen-binding portion of the antibody to binding its cognate target. The unmasking of the antigen-binding portion of the antibody provides the “conditional” nature of the binding from masked to active.

As used herein, a “subject” may be a mammalian subject. Mammalian subjects include humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

All publications, patents, and patent applications mentioned in this specification are herein incorporated by reference to the same extent as if each individual publication, patent, or patent application was specifically and individually indicated to be incorporated by reference.

### Antibodies.

As used herein, the term “antibody” refers to an intact antibody or a binding fragment thereof that binds specifically to a target antigen. Binding fragments are produced by recombinant DNA techniques, or by enzymatic or chemical cleavage of intact antibodies.

Binding fragments include Fab, Fab′, F(ab′)2, Fv, and single-chain variable fragment (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)2, Fv, scFv) so long as they exhibit the desired biological activity. Antibodies (Abs) and immunoglobulins (Igs) are glycoproteins having the same structural characteristics. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial or eukaryotic cells, including plant cells.

As used herein, the term “antibody fragment” refers to a portion of a full-length antibody, generally the antigen-binding or variable region, and include Fab, Fab′, F(ab′)2, Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)2 fragment that has two antigen-binding fragments which are capable of cross-linking antigen, and a residual other fragment (which is termed pFc′). As used herein, “functional fragment” with respect to antibodies, refers to Fv, F(ab) and F(ab′)2 fragments.

As used herein, the “Fv” fragment is the minimum antibody fragment that contains a complete antigen recognition and binding site. This region consists of a dimer of one heavy and one light chain variable domain in a tight, non-covalent association (VH-VL dimer). It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)2 pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (VH) followed by the constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.). The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for the target is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)2, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fv framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

Non-limiting examples of antigens that are the targets of the antibody include, e.g., 4-1BBL, AFP, AKAP-4, ALK, androgen receptor, bcr-abl, Clorf186, CA-125, CA6, CA6, CA19-9, CAMPATH-1, Carbonic anhydrase IX, carcinoembryonic antigen (CEA), CCR8, CD1, CD2, CD3, CD4, CD5, CD8, CD16A, CD19, CD1A, CD20, CD204, CD204, CD204, CD206, CD206, CD206, CD25, CD276, CD28, CD30, CD301, CD301, CD32A, CD32B, CD33, CD36, CD37, CD39, CD40, CD45, CD47, CD5, CD64, CD73, CEA, CLEC4C, CLDN16, CLDN6, CLDN18.2, CLEC10A, CLEC12A, CLEC5A, CLEC9A, CMET, CTCFL, cyclin B1, CYP1B1, DC-SIGN, DEC-205, Dectin1, Dectin2, DLL3, EGFR, EGFRvIII, Endoglin, endosialin, EPCAM, EphA2, epidermal growth factor, ERG, ETV6-AML, ferritin, fibroblast activation protein (FAP), FLT3, folate-binding protein, Fos-related antigen 1, FRA, fucosyl GM1, G250, GD2, GD3, Glycoprotein A33, GloboH, GLP-3, GM2, GM3, gp100, HER2, HER2/neu, HER3, HLD-DR, HMWMAA, HPV E6, HPV E7, hTERT, HVEM, IL-2 receptor, IV1, Latent-TGFB, LCK, Legumain, Ley, LIV1, LMP2, LY6E, MAD-CT-1, MAD-CT-2, MAGE A1, MAGE A3, mannose scavenger receptor1, MARCO, MelanA/MART1, mesothelin (MSLN), metalloproteinase, ML-IAP, MSLN, MUC1, MUC15, MUC16, MYCN, NA17, NAPI2B, NAPI2B, NY-BR-1, NY-ESO-1, OX40L, OY-TES1, p185HER2, P53 mutant, P53 nonmutant, PAGE4, PAP, PAX3, PAX5, PDGFR-B, PDL, PDL1, PLAV1, polysialic acid, PR1, PSA, PSCA, PSMA, PTK7, Ras mutant, RGS5, RhoC, RON, ROR1, ROR2, RRC15, SART3, SIRPA, Sperm protein 17, SSX2, STn, survivin, TAG-72, tenascin, TGFB, Tie 3, TMEM238, TMPRSS3, TMPRSS4, Tn, TRA6, TROP2, TRP-2, tyrosinase, UPK1B, vascular endothelial growth factor, VEGFR, VEGFR2, VISTA, VTCN1, WT1, XAGE 1, or Y6E.

Non-limiting examples of antibodies that can be modified to include the conditional masks of the present invention include, e.g., ABT806 (EGFRvIII), adecatumumab (EPCAM), alemtuzumab (CD33), AMG595 (EGFRvIII), anetumab (MSLN), anti-ACE2, anti-EphA2 (EphA2), anti-hyaluronidase, anti-neuraminidase, anti-NY-ESO-1, anti-PTK7 (PTK7), cetuximab (EGFR), cirmtuzumab (ROR1), clivatuzumab (MUC1), CT-011 (PD), DS-8895a variant 1 (EphA2), DS-8895a variant 2 (EphA2), durvalumab (PDL1) anti-MAGE-A3, edrecolomab (EPCAM), farletuzumab (FRA/folate receptor alpha), gemtuzumab ozogamicin (CD33), huDS6 (CA6), humanized Ab 2-3 (CEA), humanized PR1A3 (CEA), ibritumomab tiuxetan (CD52), IMAB362/claudiximab (Claudin18.2), ipilimumab (CTLA4), J591 variant 1 (PSMA), J591 variant 2 (PSMA), ladiratuzumab (LIV1), lifastuzumab (NAPI2B), MEDI-547 (EphA2), mirvetuximab (FRA), narnatumab (RON), nimotuzumab (EGFR), nivolumab (PD1), onartuzumab (c-MET), panitumumab (EGFR), patritumab (HER3), pembrolizumab (PD1), pertuzumab (HER2/neu), PF-06647020 (PTK7), RG7841 (LY6E), rituximab (CD20), rovalpituzumab (DLL3), sacituzumab (TROP2), sibrotuzumab (FAP), sofituzumab (MUC16), tositumomab (CD20), trastuzumab (HER2/neu), tremelimumab (CP-675,206)(CTLA4), or zalutumumab (EGFR).

Non-limiting examples of protease cleavage sites for use in the linker, when the linker is a peptide, are those that are cleaved by, e.g., actinidain, activated protein C, ADAM10, ADAM12, ADAMS, bromelain, bromelain, calpain, Caspase, caspase-3, Cathepsin, Cathepsin B, Cathepsin C, Cathepsin D, Cathepsin E, cathepsin G, Cathepsin K, Cathepsin L, Chymase, chymosin, chymotrypsin-like protease, CMV protease, collagenase, DESC1, dipeptidyl peptidase, dipeptidyl peptidase IV (DPPIV/CD26), disintegrin and metalloproteinase (ADAM), DPP-4, Elastase, elastase-like protease, enterokinase, Factor Xa, FAP, FAP (FAP-α), Granzyme B, Guanidinobenzoatase, Hepsin, HIV-1 protease, hK1, hK15, hK3, HSV protease, HtrAl, HumNeutrophil Elastase, interleukin-1β converting enzyme, kallikrein, kallikrein-related peptidase (KLK), Lactoferrin, legumain, Marapsin, mast cell chymase, mast cell tryptase, matriptase, Matriptase-2, matrix metalloprotease (MMP), metalloendopeptidase, metalloexopeptidase, Mirl-CP, metalloproteases (MMP1, MMP2, MMP3, MMP7, MMP8, MMP9, MMP10, MMP11, MMP12, MMP13, MMP14), nepenthesin, neutrophil elastase, neutrophil serine protease 4, NS3/4A, PACE4, papain, pepsin, plasmepsin, plasmin, prostate-specific antigen (PSA), proteinase 3, renin, secretase, stromelysin, subtilisin-like protease, thrombin, tissue plasminogen activator (tPA), transmembrane Serine Protease (TMPRSS), trypsin, trypsin-like protease, tryptase, type II transmembrane serine protease (TTSP), Type IV collagenase, urokinase plasminogen activator (uPA), or urokinase plasminogen activator receptor (uPAR).

Non-amino acid cleavage polymers or linkers can include, e.g., nucleic acids, lipids, carbohydrates, or chemical linkers, such as those that are cleaved or dissociated by, e.g., light, radiation, electromagnetic, pH, chemically, or enzymatically.

Structural-switch linkers can include amino acid or non-amino acid polymers that adopt an inactivated prodrug conformation with the mask blocking the antibody-antigen binding site. Upon exposure to specific microenvironment conditions such as pH, enzyme(s), or metabolite(s)—the linker switches to the activated antibody conformation where the mask no longer blocks antibody-antigen binding.

As used herein, the term “treatment” refers to both therapeutic treatment and prophylactic or preventative measures. Those in need of treatment include those already with the disorder as well as those in which the disorder is to be prevented.

As used herein, the term “disorder” refers to any condition that would benefit from treatment with the polypeptide. This includes chronic and acute disorders or diseases including those infectious or pathological conditions that predispose the mammal to the disorder in question.

An antibody or antibody fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in WO/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved Clq binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “epitope” refers to a specific amino acid sequence or molecule (such as a carbohydrate, small molecule, lipid, etc.) that when present in the proper conformation, provides a reactive site for an antibody (e.g., B cell epitope) or in the case of a peptide to a T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715.

As used herein, the term “substantially purified” refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

As used herein, a “target” may be an antigen (or putative antigen) from a pathogenic organism; a protein or carbohydrate involved in cellular functions associated with disease; an enzyme; a signaling molecule; or any protein, lipid, nucleic acid, or other molecular structure to which an antibody will biding. The target may be an engineered polypeptide recapitulating a portion of a desired target protein, nucleic acid, carbohydrate, lipid, etc. A “target” may, in a variation, be more than one protein, such as a multimeric protein complex, nucleic acid, carbohydrate, lipid, etc. For simplicity, the disclosure refers to a target, but the methods apply to multimeric structures as well. In a variation, a target protein is a single, or two or more distinct proteins or protein complexes. For example, the methods disclosed herein may be used to design engineered peptides, nucleic acids, carbohydrates, lipids, etc., that mimic common attributes of targets from diverse species—e.g., to target a conserved epitope for antibody selection.

A computational record of the topology of the protein is derived, termed here a “reference target structure.” The reference target structure may be a conventional protein structure or a structural model, represented for example by 3D coordinates for all (or most) atoms in the protein or 3D coordinates for select atoms (e.g., coordinates of the CP atoms of each protein residue). Optionally the reference target structure may include dynamic terms derived either computationally (e.g., from molecular dynamics simulation) or experimentally (e.g., from spectroscopy, crystallography, or electron microscopy).

The predetermined portion of the target protein is converted into a blueprint having target-residue positions and scaffold-residue positions. Each position may be assigned either a fixed amino-acid residue identity or a variable identity (e.g., any amino acid, or an amino acid with desired physiochemical properties—polar/non-polar, hydrophobicity, size, etc.). In a variation, each amino acid from the predetermined portion of the target protein is mapped to one target-residue position, which is assigned to have the same amino-acid identity as found in the target protein. The target-residue positions may be continuous and/or ordered. An advantage, however, in some variations, is that the target-residue position may be discontinuous (interrupted by scaffold-residue positions) and not ordered (in a different order from the target protein). Unlike grafting approaches, in some variations, the order of residues is not constrained. Similarly, the disclosed methods can accommodate discontinuous portions of the target protein (e.g., discontinuous epitopes where different portions of the same protein or even different protein chains contribute to one epitope).

The scaffold-residue positions of the blueprint may be assigned to have any amino acid at that position (i.e., an X representing any amino acid). In variations, the scaffold-residue position is assigned by selection from a subset of possible natural or unnatural amino acids (e.g., small polar amino acid residue, large hydrophobic amino-acid residue, etc.). The blueprint may also accommodate optional target- and/or scaffold-residue positions. Similarly stated, the blueprint may tolerate insertion or deletion of residue positions. For example, a target- or scaffold-residue position may be assigned to be present or absent; or the position may be assigned to be 0, 1, 2, 3, 4, 5, 6, 7, 8, 9, 10, or more residues.

A subset of the blueprints may then be used to perform computational modeling to generate corresponding polypeptide structures, using, e.g., energy terms(s) and topological constraint(s) derived from the reference target structure, with a score calculated for each polypeptide structure. A machine learning (ML) model may be trained using the scores and the blueprints, or representations of the blueprints (e.g., vectors that represent the blueprints), and the ML model may be executed to generate further blueprints. An advantage of this method is that the topological space covered by vastly more blueprints may be explored by the ML model than could be explored by iterative computational modeling of many blueprints.

The disclosure further provides methods and related devices to convert output blueprints to sequences and/or structures of engineered polypeptides, and to compare these engineered polypeptides to the target protein—using static comparison, dynamic comparison or both—and to filter the polypeptides using these comparisons.

### Engineered Epitope Mask Design.

Peptide scaffolds were computationally designed to support the native epitope sequence and structure with the iBio Engineered Epitope machine learning engine. The design process starts with an initial set of blueprints indicating the positions of the scaffold residues to design and the positions of the epitope residues to hold fixed. The machine learning engine optimizes the scaffold residues in these initial blueprints for [1] structural match to the native epitope structure, [2] overall structural stability of the molecule, and [3] solubility of the peptide design. The number of scaffold residues in the blueprints are then iteratively reduced until the machine learning engine can no longer satisfy all three of the loss functions described above. The final design is based on the blueprint that satisfies all loss functions using the minimal number of scaffold residues.

Engineered epitopes are then tested for binding to their corresponding antibodies by Octet measurements. Binders are identified as potential candidates for use in antibody masking.

### Masked Ab Expression & Purification.

Masked and unmasked antibodies were expressed in either IgG or scFv format using the HEK293, CHO, or ExpiCHO cell lines. Cell supernatant was collected by centrifugation of the mammalian cells at 300 g followed by centrifugation at 2000 g. Antibodies were purified by either Protein A or Protein G column chromatography depending upon the isotype. Antibodies were eluted in pH 3 citrate and passed through a desalting column to switch the buffer to pH 7.4 phosphate buffered saline.

### Proteolytic Cleavage.

Masks were proteolytically cleaved from the antibody with Matrix Metallopeptidase 9 (MMP9). Pre-activated MMP9 (Sigma catalogue #SAE0078) was added at a 1:100 weight ratio to 2 mg/mL masked antibody in phosphate buffered saline. The reaction was incubated overnight at room temperature.

### BLI and Flow Cytometry Binding.

Octet Binding Kinetics. Antibody binding kinetics were measured by bio-layer interferometry on an Octet. The target antigen was loaded onto a biosensor tip coated in anti-penta-histidine antibody, and the masked antibody test articles were flowed over the tip at a concentration of 50 nM. For each masked antibody, binding kinetics were measured for three samples: [1] antibody without a mask, [2] antibody with a mask, and [3] antibody with a mask+MMP9 treatment.

Flow Cytometry. Antibody binding to the target antigen expressed on cells was measured with flow cytometry. Antibodies were incubated with cells transiently expressing the target antigen for 1 hour, washed once with DPBS+1% FBS, then labeled with anti-mouse IgG secondary antibody for 1 hour. Cells were then washed again and measured by flow cytometry. All measurements were performed on ice and with chilled buffers.

The skilled artisan will recognize that antibodies which exhibit little or no binding to a target antigen can be described as having a low affinity, and a high equilibrium dissociation constant (KD) for the target antigen. The skilled artisan will also recognize that antibodies which exhibit little or no binding to a collective assembly of target antigenic epitopes can be described as having a low avidity, and a high equilibrium dissociation constant (KD) for the collective assembly of target antigenic epitopes.

### Example. 1. Masked, Conditionally Active, Anti-CD3 Antibodies—Sequences

Provided herein are sequences for exemplary masked, conditionally active, anti-CD3 antibodies of the disclosure. Included are the mask region, the linker, and the antibody including the complementarity determining region (CDR) sequences and the variable heavy and light domain sequences (VH, VL) that constitute the anti-CD3 antigen binding sites of the masked antibodies of the disclosure.

TABLE 1
Exemplary Masked Anti-CD3 Antibody CDR.

| HCDR1 | HCDR2 | HCDR3 | LCDR1 | LCDR2 | LCDR3 |
|---|---|---|---|---|---|
| GYSFTGYT | INPYKGVS | ARSGYYGDSDWYFDV | QDIRNY | YTS | QQGNTLPWT |
| SEQ ID | SEQ ID | SEQ ID NO: 3 | SEQ ID | SEQ ID | SEQ ID NO: 6 |
| NO: 1 | NO: 2 | | NO: 4 | NO: 5 | |

TABLE 2
Exemplary Masked Anti-CD3 Antibodies.

| UCHT1 long linker mask on VH domain | VL Domain |
|---|---|
| CPQYPGSEILWQKWGNKKVCYPRGSKPEDAN | DIQMTQSPSSLSASVGDRVTITCRASQDIRNYLN |
| GGGGSSGGPGPAGMKGLPGSEVQLVESGGGL | WYQQKPGKAPKLLIYYTSRLESGVPSRFSGSGSG |
| VQPGGSLRLSCAASGYSFTGYTMNWVRQAPGK | TDYTLTISSLQPEDFATYYCQQGNTLPWTFGQGT |
| GLEWVALINPYKGVSTYNQKFKDRFTISVDKSKN | KVEIK |
| TAYLQMNSLRAEDTAVYYCARSGYYGDSDWYF | |
| DVWGQGTLVTVSS | |
| SEQ ID NO: 7 | SEQ ID NO: 8 |
| Bold = Engineered Epitope | VL Domain |
| Underlined = Spacer | |
| Italics = Cleavage site | |
| Double Underline = VH domain | |
| VH Domain | UCHT1 long linker mask on VL domain |
| EVQLVESGGGLVQPGGSLRLSCAASGYSFTGYT | CPQYPGSEILWQKWGNKKVCYPRGSKPEDAN |
| MNWVRQAPGKGLEWVALINPYKGVSTYNQKF | GGGGSSGGPGPAGMKGLPGSDIQMTQSPSSLS |
| KDRFTISVDKSKNTAYLQMNSLRAEDTAVYYCA | ASVGDRVTITCRASQDIRNYLNWYQQKPGKAPK |
| RSGYYGDSDWYFDVWGQGTLVTVSS | LLIYYTSRLESGVPSRFSGSGSGTDYTLTISSLQPED |
| | FATYYCQQGNTLPWTFGQGTKVEIK |
| SEQ ID NO: 9 | SEQ ID NO: 10 |
| VH Domain | Bold = Engineered Epitope |
| | Underlined = Spacer |
| | Italics = Cleavage site |
| | Double Underline = VL domain |

TABLE 3
Exemplary Variable Heavy Chain and Variable Light Chain Nucleic Acid
Sequences of Masked anti-CD3 Antibodies.

| Antibody | Variable Heavy Chain | SEQ ID | Variable Light Chain | SEQ ID |
|---|---|---|---|---|
| ID | Nucleic Acid Sequence | NO: | Nucleic Acid Sequence | NO: |
| UCHT1 | tgcccccagtaccccggctccga | 11 | gacatccagatgacccagtccccctcctc | 12 |
| long linker | gatcctgtggcagaagtggggca | | cctgtccgcctccgtgggcgacagggtga | |
| mask on | acaagaaggtgtgctaccccagg | | ccatcacctgcagggcctcccaggacatc | |
| VH domain | ggctccaagcccgaggacgccaa | | aggaactacctgaactggtaccagcaga | |
| | cggcggcggcggctcctccggcg | | agcccggcaaggcccccaagctgctgat | |
| | gccccggccccgccggcatgaag | | ctactacacctccaggctggagtccggcg | |
| | ggcctgcccggctccgaggtgca | | tgccctccaggttctccggctccggctccg | |
| | gctggtggagtccggcggcggcc | | gcaccgactacaccctgaccatctcctcc | |
| | tggtgcagcccggcggctccctg | | ctgcagcccgaggacttcgccacctacta | |
| | aggctgtcctgcgccgcctccggc | | ctgccagcagggcaacaccctgccctgg | |
| | tactccttcaccggctacaccatg | | accttcggccagggcaccaaggtggaga | |
| | aactgggtgaggcaggcccccgg | | tcaag | |
| | caagggcctggagtgggtggccc | | | |
| | tgatcaacccctacaagggcgtgt | | | |
| | ccacctacaaccagaagttcaag | | | |
| | gacaggttcaccatctccgtggac | | | |
| | aagtccaagaacaccgcctacct | | | |
| | gcagatgaactccctgagggccg | | | |
| | aggacaccgccgtgtactactgc | | | |
| | gccaggtccggctactacggcga | | | |
| | ctccgactggtacttcgacgtgtg | | | |
| | gggccagggcaccctggtgaccg | | | |
| | tgtcctcc | | | |
| UCHT1 | gaggtgcagctggtggagtccgg | 13 | tgcccccagtaccccggctccgagatcct | 14 |
| long linker | cggcggcctggtgcagcccggcg | | gtggcagaagtggggcaacaagaaggt | |
| mask on VL | gctccctgaggctgtcctgcgccg | | gtgctaccccaggggctccaagcccgag | |
| domain | cctccggctactccttcaccggct | | gacgccaacggcggcggcggctcctccg | |
| | acaccatgaactgggtgaggcag | | gcggccccggccccgccggcatgaaggg | |
| | gcccccggcaagggcctggagtg | | cctgcccggctccgacatccagatgaccc | |
| | ggtggccctgatcaacccctaca | | agtccccctcctccctgtccgcctccgtgg | |
| | agggcgtgtccacctacaaccag | | gcgacagggtgaccatcacctgcagggc | |
| | aagttcaaggacaggttcaccat | | ctcccaggacatcaggaactacctgaact | |
| | ctccgtggacaagtccaagaaca | | ggtaccagcagaagcccggcaaggcccc | |
| | ccgcctacctgcagatgaactccc | | caagctgctgatctactacacctccaggc | |
| | tgagggccgaggacaccgccgtg | | tggagtccggcgtgccctccaggttctcc | |
| | tactactgcgccaggtccggctac | | ggctccggctccggcaccgactacaccct | |
| | tacggcgactccgactggtacttc | | gaccatctcctccctgcagcccgaggact | |
| | gacgtgtggggccagggcaccct | | tcgccacctactactgccagcagggcaac | |
| | ggtgaccgtgtcctcc | | accctgccctggaccttcggccagggcac | |
| | | | caaggtggagatcaag | |

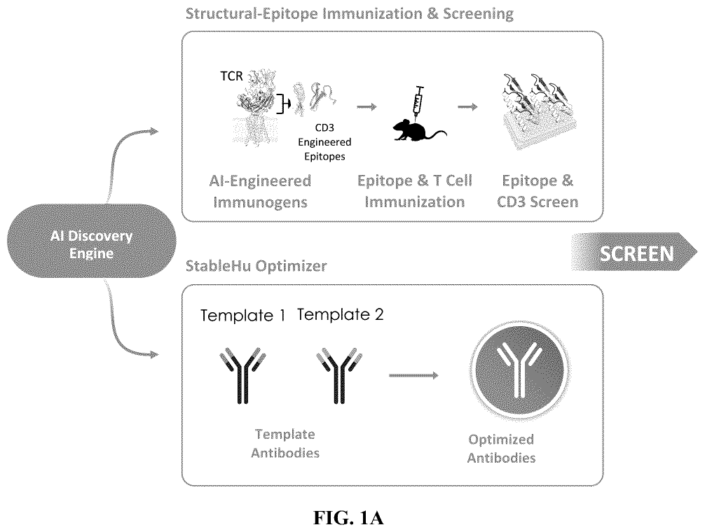

FIGS. 1A and 1B illustrate the dual approaches used to generate a diverse panel of anti-CD3 antibodies. FIG. 1A shows the use of the AI discovery engine applied to either a structure-epitope immunization and screen, or the use of a StableHu optimizer. FIG. 1B shows that the results of the structure-epitope immunization and screen, or the use of a StableHu optimizer are then screened for binding or T cell activation.

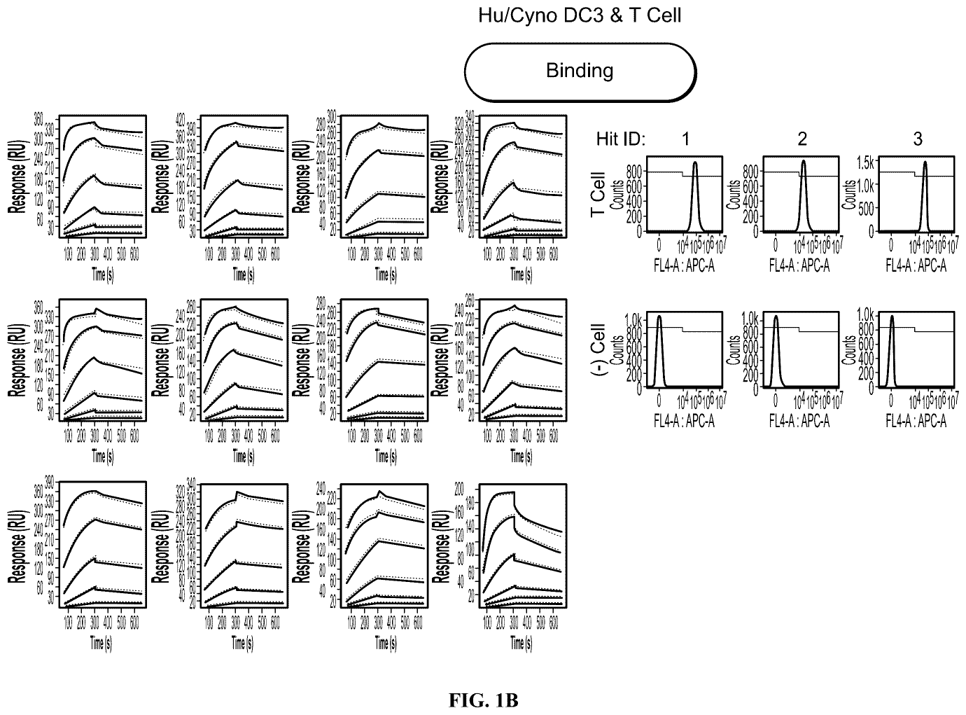

FIG. 2 shows that the engineered epitopes guide immunization to TCR-accessible CD3 epitopes and are potential masks for conditionally-activated antibodies.

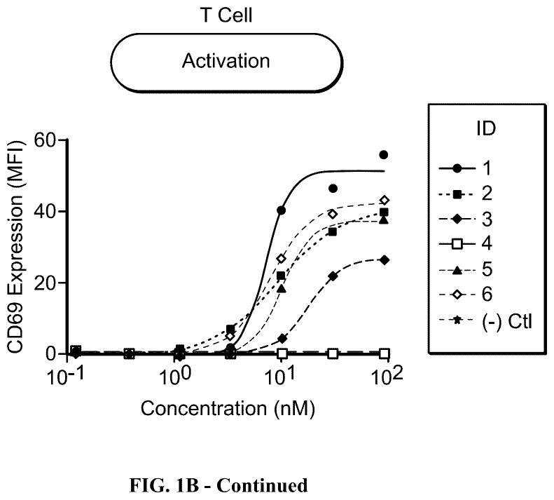

FIG. 3A is a graph showing that engineered epitope 1 is a conditionally-activated mask for anti-CD3 clone UCHT1, measuring engineered epitope 1 binding to UCHT1.

FIG. 3B is a graph showing that engineered epitope 1 is a conditionally-activated mask for anti-CD3 clone UCHT1 which shows the conditionally-activated UCHT1 Ab binding to T cells.

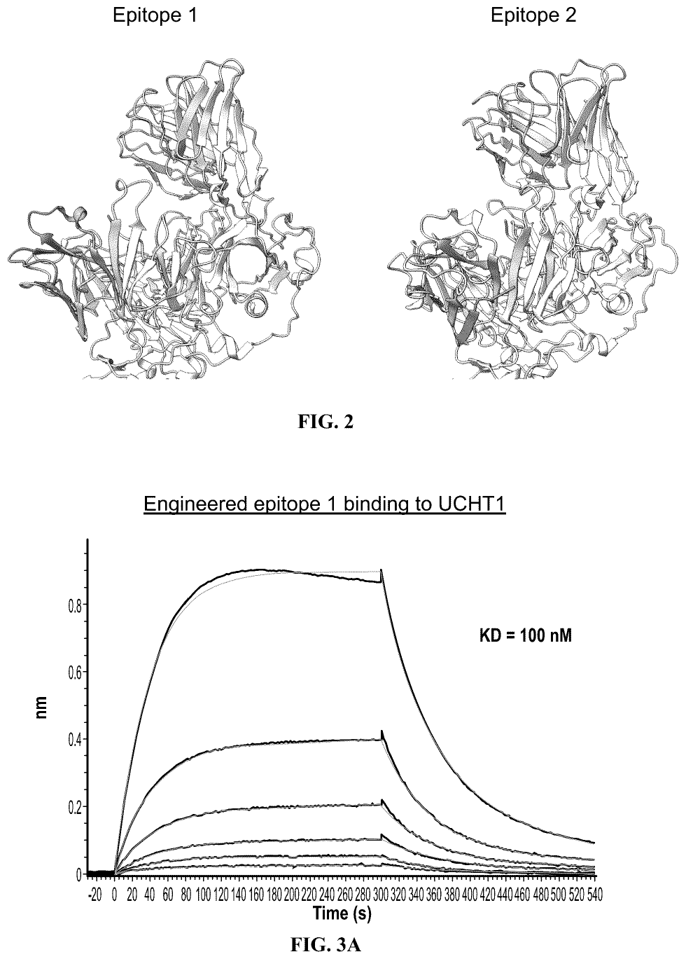

FIG. 4 shows that engineered epitope 2 is a conditionally-activated mask for anti-CD3 clones SP34 and 6-F05.

### Example. 2. Masked, Conditionally Active, Anti-MUC16 Antibodies—Sequences

Provided herein are sequences for exemplary masked, conditionally active, anti-MUC16 antibodies of the disclosure. Included are the mask region, the linker, and the antibody including the complementarity determining region (CDR) sequences and the variable heavy and light domain sequences (VH, VL) that constitute the anti-CD3 antigen binding sites of the masked antibodies of the disclosure.

TABLE 4
Exemplary Masked Anti-MUC16 Antibody CDR.

| HCDR1 | HCDR2 | HCDR3 | LCDR1 | LCDR2 | LCDR3 |
|---|---|---|---|---|---|
| GYSITSDYA | ISYSGST | ATLGLDY | QSLLYSSNQKNY | WAS | QQYHSYRT |
| SEQ ID | SEQ ID | SEQ ID | SEQ ID NO: 18 | SEQ ID | SEQ ID |
| NO: 15 | NO: 16 | NO: 17 | | NO: 19 | NO: 20 |

TABLE 5
Exemplary Masked Anti-MUC16 Antibodies.

| 21G6 long linker mask on VH domain | VL Domain |
|---|---|
| NFTLDRSSVLVDGYSPNRNEPLTGNSDLPGGG | DIVMTQSPDSLAVSLGERATINCKSSQSLLYSSNQ |
| GSSGGPGPAGMKGLPGSQVQLQESGPGLVKP | KNYLAWYQQKPGQPPKLLIYWASTRGSGVPDRF |
| SQTLSLTCTVSGYSITSDYAWNWIRQPPGKGLE | SGSGSGTDFTLTISSLQAEDVAVYYCQQYHSYRTF |
| WIGYISYSGSTIYNPSLKSRISISVDTSKNQFSLKL | GQGTKVEIK |
| NSVTAADTAVYYCATLGLDYWGQGTLVTVSS | |
| SEQ ID NO: 21 | SEQ ID NO: 22 |
| Bold = Engineered Epitope | VL Domain |
| Underlined = Spacer | |
| Italics = Cleavage site | |
| Double Underline = VH domain | |
| VH Domain | 21G6 short linker mask on VL domain |
| QVQLQESGPGLVKPSQTLSLTCTVSGYSITSDYA | NFTLDRSSVLVDGYSPNRNEPLTGNSDLPSGGPG |
| WNWIRQPPGKGLEWIGYISYSGSTIYNPSLKSRI | PAGMKGLPGSDIVMTQSPDSLAVSLGERATINCK |
| SISVDTSKNQFSLKLNSVTAADTAVYYCATLGLD | SSQSLLYSSNQKNYLAWYQQKPGQPPKLLIYWAS |
| YWGQGTLVTVSS | TRGSGVPDRFSGSGSGTDFTLTISSLQAEDVAVYY |
| | CQQYHSYRTFGQGTKVEIK |
| SEQ ID NO: 23 | SEQ ID NO: 24 |
| VH Domain | Bold = Engineered Epitope |
| | Underlined = Spacer |
| | Italics = Cleavage site |
| | Double Underline = VL domain |

TABLE 6
Exemplary Variable Heavy Chain and Variable Light Chain Nucleic Acid
Sequences of Masked anti-MUC16 Antibodies.

| | | SEQ | | SEQ |
|---|---|---|---|---|
| Antibody | Variable Heavy Chain Nucleic | ID | Variable Light Chain Nucleic | ID |
| ID | Acid Sequence | NO: | Acid Sequence | NO: |
| 21G6 | aacttcaccctggacaggtcctccgtgc | 25 | gacatcgtgatgacccagtcccccgactccctg | 26 |
| long | tggtggacggctactcccccaacagga | | gccgtgtccctgggcgagagggccaccatcaa | |
| linker | acgagcccctgaccggcaactccgacc | | ctgcaagtcctcccagtccctgctgtactcctcc | |
| mask on | tgcccggcggcggcggctcctccggcg | | aaccagaagaactacctggcctggtaccagca | |
| VH | gccccggccccgccggcatgaagggcc | | gaagcccggccagccccccaagctgctgatct | |
| domain | tgcccggctcccaggtgcagctgcagg | | actgggcctccaccaggggctccggcgtgccc | |
| | agtccggccccggcctggtgaagccctc | | gacaggttctccggctccggctccggcaccgac | |
| | ccagaccctgtccctgacctgcaccgtg | | ttcaccctgaccatctcctccctgcaggccgag | |
| | tccggctactccatcacctccgactacg | | gacgtggccgtgtactactgccagcagtaccac | |
| | cctggaactggatcaggcagccccccg | | tcctacaggaccttcggccagggcaccaaggt | |
| | gcaagggcctggagtggatcggctaca | | ggagatcaag | |
| | tctcctactccggctccaccatctacaac | | | |
| | ccctccctgaagtccaggatctccatctc | | | |
| | cgtggacacctccaagaaccagttctcc | | | |
| | ctgaagctgaactccgtgaccgccgcc | | | |
| | gacaccgccgtgtactactgcgccaccc | | | |
| | tgggcctggactactggggccagggca | | | |
| | ccctggtgaccgtgtcctcc | | | |
| 21G6 | caggtgcagctgcaggagtccggcccc | 27 | aacttcaccctggacaggtcctccgtgctggtg | 28 |
| short | ggcctggtgaagccctcccagaccctgt | | gacggctactcccccaacaggaacgagcccct | |
| linker | ccctgacctgcaccgtgtccggctactc | | gaccggcaactccgacctgccctccggcggcc | |
| mask on | catcacctccgactacgcctggaactgg | | ccggccccgccggcatgaagggcctgcccggc | |
| VL | atcaggcagccccccggcaagggcctg | | tccgacatcgtgatgacccagtcccccgactcc | |
| domain | gagtggatcggctacatctcctactccg | | ctggccgtgtccctgggcgagagggccaccatc | |
| | gctccaccatctacaacccctccctgaa | | aactgcaagtcctcccagtccctgctgtactcct | |
| | gtccaggatctccatctccgtggacacc | | ccaaccagaagaactacctggcctggtaccag | |
| | tccaagaaccagttctccctgaagctga | | cagaagcccggccagccccccaagctgctgat | |
| | actccgtgaccgccgccgacaccgccg | | ctactgggcctccaccaggggctccggcgtgcc | |
| | tgtactactgcgccaccctgggcctgga | | cgacaggttctccggctccggctccggcaccga | |
| | ctactggggccagggcaccctggtgac | | cttcaccctgaccatctcctccctgcaggccga | |
| | cgtgtcctcc | | ggacgtggccgtgtactactgccagcagtacca | |
| | | | ctcctacaggaccttcggccagggcaccaagg | |
| | | | tggagatcaag | |

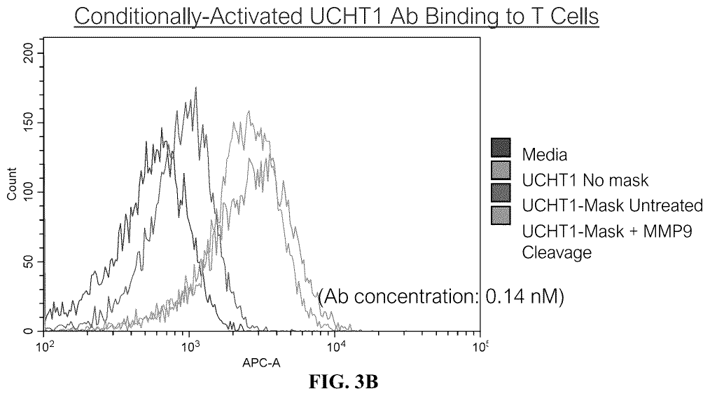

FIG. 5 shows the method of immunizations steered to a MUC16 epitope that avoids epitope shedding.

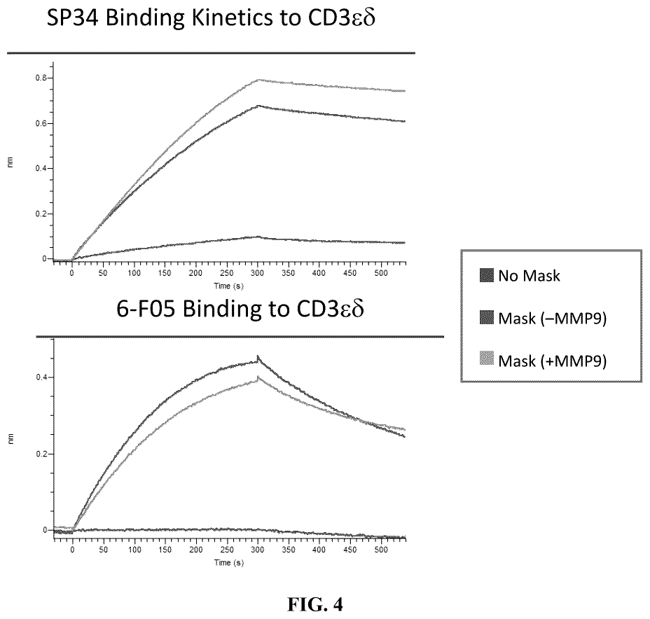

FIG. 6 summarizes MUC16 engineered epitope is a cleavable mask for the 21G6 Anti-MUC16 antibody.

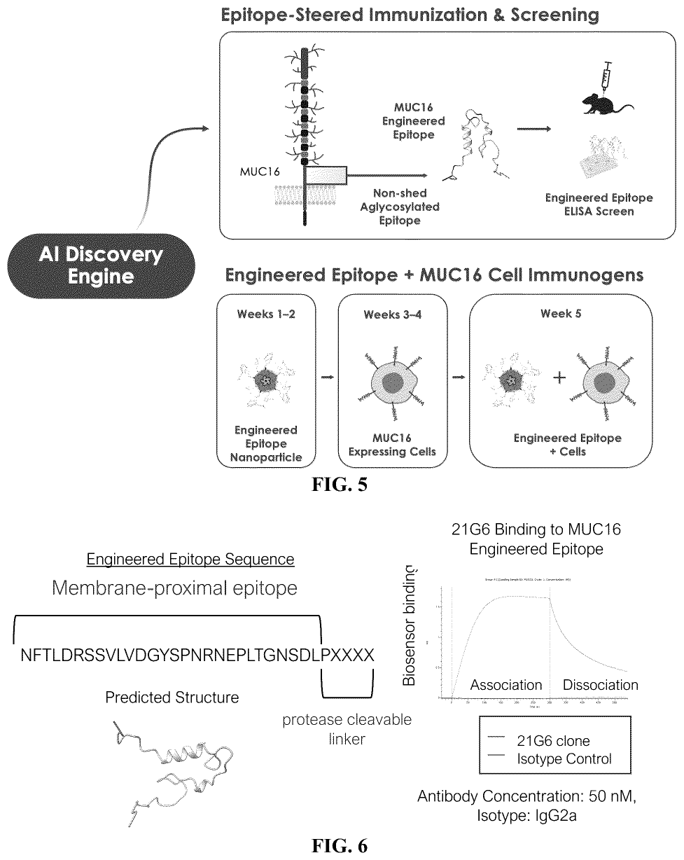

FIGS. 7A and 7B show that the conditionally-activated 21G6 Anti-MUC16 Clone Masked with engineered epitope only binds MUC16 membrane-proximal region after protease treatment. FIG. 11A shows the results from the construct with the long linker mask on VH domain. FIG. 11B shows the results from the construct with the long linker mask on VL domain.

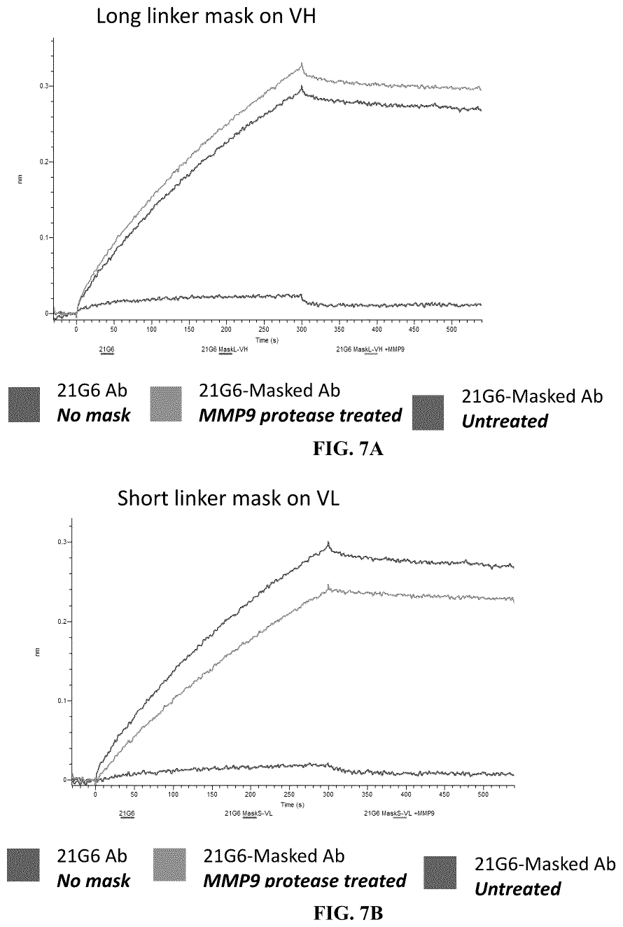

FIG. 8 shows the results from a CD3 epitope masking construct by ELISA. CD3 ELISA: Coat plate with antibody, detect with RP-labeled CD3. A greater than 100-fold shift observed when comparing the masked CD3 antibody before (hexagon) and after (square) digestion with MMP9.

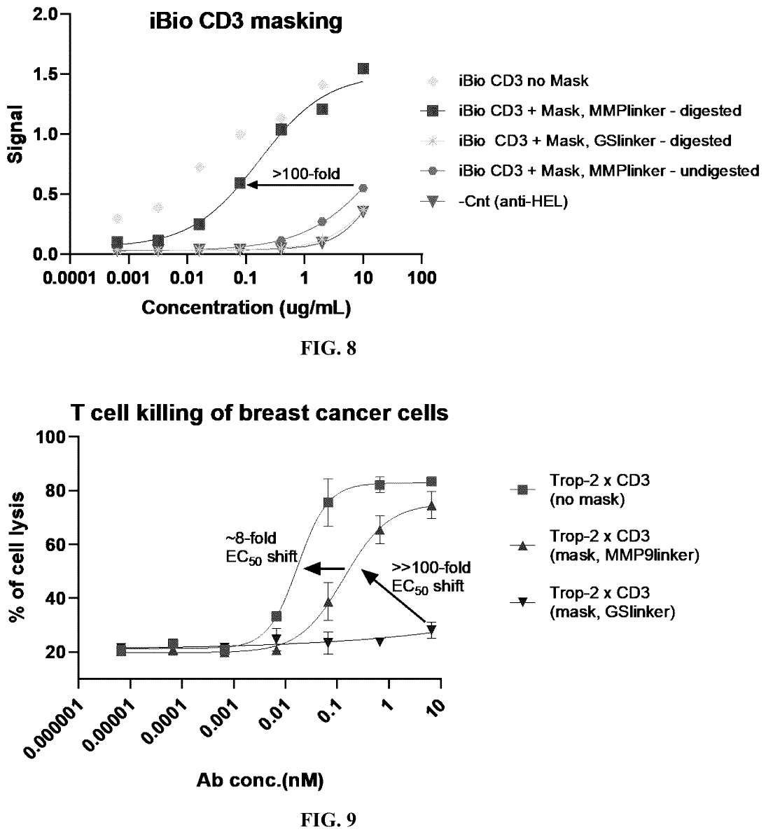

FIG. 9 shows T cell killing with masked CD3 bispecific. PBMC coculture with MDA-MB-231 breast cancer cell line. Antibodies added undigested, MMP9/other proteases produced by cancer cells. Bispecific with CD3 arm masked and cleavable linker had >>100-fold greater cell killing potency than the same molecule with an uncleavable linker, and was within 10-fold potency of the unmasked variant of the bispecific.

In some embodiments, the antibodies provided herein are useful for the treatment of an autoimmune disease. An autoimmune disease consists of a potentially harmful immune response to a self-antigen. Examples of autoimmune diseases include: alopecia, ankylosing spondylitis, atopic dermatitis, celiac disease, Crohn's disease, cutaneous lupus erythematosus (CLE), lupus nephritis, multiple sclerosis, neuromyelitis optica, psoriasis, psoriatic arthritis, rheumatoid arthritis, scleroderma, Sjogren's syndrome, systemic lupus, systemic lupus erythematosus (SLE), temporal arteritis, type I diabetes, ulcerative colitis, uveitis, and vitiligo.

In some embodiments, the antibodies provided herein are useful for the treatment of a hyperinflammatory disease. A hyperinflammatory disease consists of a potentially harmful overstimulated immune response. Examples of hyperinflammatory diseases include: chronic allergy, hypersensitivity vasculitis, and T cell hypersensitivity disease.

In some embodiments, the antibodies provided herein are useful for the treatment of cancer selected from acute lymphoblastic leukemia, acute myelogenous leukemia, adrenal glands cancer, and combinations thereof., bladder tumor, bone marrow cancer, breast cancer, chronic lymphocytic leukemia, chronic myeloid leukemia, central nervous system cancer, colorectal cancer, colorectal carcinoma, endometrial cancer, endometrial carcinoma, gastric cancer, gut carcinoma, head and neck squamous cell carcinoma, hematopoietic malignancies, liver cancer, lung carcinoma, lymph node cancer, melanoma, metastatic colorectal cancer, ovarian cancer, pancreatic adenocarcinoma, pituitary tumor, prostate cancer, prostate carcinoma, renal cell carcinoma, retinal cancer, sarcoma, skin cancer, spleen cancer, stomach cancer, thymus cancer, and/or thyroid cancer.

### Administration of Therapeutic Antibodies.

The in vivo administration of the therapeutic antibodies described herein may be carried out intravenously, intramuscularly, subcutaneously, topically, orally, transdermally, intraperitoneally, intraorbitally, intrathecally, intraventricularly, intranasally, transmucosally, through implantation, or through inhalation. Intravenous administration may be carried out via injection or infusion. In some embodiments, the epitope-targeted, conditionally-activated, pro-drug, antibody of the disclosure are administered intravenously. In some embodiments, the epitope-targeted, conditionally-activated, pro-drug, antibody of the disclosure are administered subcutaneously. Administration of the therapeutic epitope-targeted, conditionally-activated, pro-drug, antibody may be performed with any suitable excipients, carriers, or other agents to provide suitable or improved tolerance, transfer, delivery, and the like.

### Additional CDR Sequences.

The CDRs of antibodies in the tables below can be veneered or engineered into an antibody framework such as those disclosed herein above. The 6 CDRs (3 heavy chain and 3 light chain) generally match those of the matching set from those taught below for each of the clones, and can be engineered to include the engineered epitope and linker (linker) as taught herein above.

TABLE 7
Anti-CD3-Heavy Chain CDRs.

| | | SEQ | | SEQ | | SEQ |
|---|---|---|---|---|---|---|
| | | ID | | ID | | ID |
| Clone ID | HCDR1 | NO: | HCDR2 | NO: | HCDR3 | NO: |
| SP34-H1- | GYTFNIYS | 29 | IRSKYNNYA | 30 | VRHGNFGNSYVS | 31 |
| StableHu- | | | T | | WFAY | |
| 1 | | | | | | |
| SP34-H1- | GFGFNAYA | 32 | IRSKYNNYA | 33 | VRHGNFGNSYVS | 34 |
| StableHu- | | | T | | WFAY | |
| 2 | | | | | | |
| SP34-H1- | RFTFETYA | 35 | IRSKYNNYA | 36 | VRHGNFGNSYVS | 37 |
| StableHu- | | | T | | WFAY | |
| 3 | | | | | | |
| SP34-H1- | GFAFNAYA | 38 | IRSKYNNYA | 39 | VRHGNFGNSYVS | 40 |
| StableHu- | | | T | | WFAY | |
| 4 | | | | | | |
| SP34-H1- | DFTFETYA | 41 | IRSKYNNYA | 42 | VRHGNFGNSYVS | 43 |
| StableHu- | | | T | | WFAY | |
| 5 | | | | | | |
| SP34-H1- | GFAFNVYA | 44 | IRSKYNNYA | 45 | VRHGNFGNSYVS | 46 |
| StableHu- | | | T | | WFAY | |
| 6 | | | | | | |
| SP34-H1- | GFDFNAYA | 47 | IRSKYNNYA | 48 | VRHGNFGNSYVS | 49 |
| StableHu- | | | T | | WFAY | |
| 7 | | | | | | |
| SP34-H1- | GFTFNIQA | 50 | IRSKYNNYA | 51 | VRHGNFGNSYVS | 52 |
| StableHu- | | | T | | WFAY | |
| 8 | | | | | | |
| SP34-H1- | GFTFETYA | 53 | IRSKYNNYA | 54 | VRHGNFGNSYVS | 55 |
| StableHu- | | | T | | WFAY | |
| 9 | | | | | | |
| SP34-H1- | GFSFVTYA | 56 | IRSKYNNYA | 57 | VRHGNFGNSYVS | 58 |
| StableHu- | | | T | | WFAY | |
| 10 | | | | | | |
| SP34-H1- | EFTFETYA | 59 | IRSKYNNYA | 60 | VRHGNFGNSYVS | 61 |
| StableHu- | | | T | | WFAY | |
| 11 | | | | | | |
| SP34-H1- | GFGFNVYA | 62 | IRSKYNNYA | 63 | VRHGNFGNSYVS | 64 |
| StableHu- | | | T | | WFAY | |
| 12 | | | | | | |
| SP34-H1- | GFTFYTYI | 65 | IRSKYNNYA | 66 | VRHGNFGNSYVS | 67 |
| StableHu- | | | T | | WFAY | |
| 13 | | | | | | |
| SP34-H1- | GYTFNVYA | 68 | IRSKYNNYA | 69 | VRHGNFGNSYVS | 70 |
| StableHu- | | | T | | WFAY | |
| 14 | | | | | | |
| SP34-H1- | GFTFNPYA | 71 | IRSKYNNYA | 72 | VRHGNFGNSYVS | 73 |
| StableHu- | | | T | | WFAY | |
| 15 | | | | | | |
| SP34-H1- | GFTFTTYA | 74 | IRSKYNNYA | 75 | VRHGNFGNSYVS | 76 |
| StableHu- | | | T | | WFAY | |
| 16 | | | | | | |
| SP34-H2- | GFTFNTYA | 77 | IRSKRSNYA | 78 | VRHGNFGNSYVS | 79 |
| StableHu- | | | T | | WFAY | |
| 1 | | | | | | |
| SP34-H2- | GFTFNTYA | 80 | IRSKGNNYA | 81 | VRHGNFGNSYVS | 82 |
| StableHu- | | | T | | WFAY | |
| 2 | | | | | | |
| SP34-H2- | GFTFNTYA | 83 | IRTKSNNYA | 84 | VRHGNFGNSYVS | 85 |
| StableHu- | | | V | | WFAY | |
| 3 | | | | | | |
| SP34-H2- | GFTFNTYA | 86 | IKTKHDNYA | 87 | VRHGNFGNSYVS | 88 |
| StableHu- | | | T | | WFAY | |
| 4 | | | | | | |
| SP34-H2- | GFTFNTYA | 89 | IRSKNFNYA | 90 | VRHGNFGNSYVS | 91 |
| StableHu- | | | T | | WFAY | |
| 5 | | | | | | |
| SP34-H2- | GFTFNTYA | 92 | VRSKQTNYA | 93 | VRHGNFGNSYVS | 94 |
| StableHu- | | | T | | WFAY | |
| 6 | | | | | | |
| SP34-H2- | GFTFNTYA | 95 | IRSKHNNYE | 96 | VRHGNFGNSYVS | 97 |
| StableHu- | | | T | | WFAY | |
| 7 | | | | | | |
| SP34-H2- | GFTFNTYA | 98 | IRSYINNYAT | 99 | VRHGNFGNSYVS | 100 |
| StableHu- | | | | | WFAY | |
| 8 | | | | | | |
| SP34-H2- | GFTFNTYA | 101 | IRSKVRNYA | 102 | VRHGNFGNSYVS | 103 |
| StableHu- | | | T | | WFAY | |
| 9 | | | | | | |
| SP34-H2- | GFTFNTYA | 104 | VRSKVNNYA | 105 | VRHGNFGNSYVS | 106 |
| StableHu- | | | T | | WFAY | |
| 10 | | | | | | |
| SP34-H2- | GFTFNTYA | 107 | IRSKSGNYA | 108 | VRHGNFGNSYVS | 109 |
| StableHu- | | | T | | WFAY | |
| 11 | | | | | | |
| SP34-H2- | GFTFNTYA | 110 | IRSKPNEYA | 111 | VRHGNFGNSYVS | 112 |
| StableHu- | | | T | | WFAY | |
| 12 | | | | | | |
| SP34-H2- | GFTFNTYA | 113 | IRSKFKNYA | 114 | VRHGNFGNSYVS | 115 |
| StableHu- | | | T | | WFAY | |
| 13 | | | | | | |
| SP34-H2- | GFTFNTYA | 116 | IRSKYTNYA | 117 | VRHGNFGNSYVS | 118 |
| StableHu- | | | T | | WFAY | |
| 14 | | | | | | |
| SP34-H2- | GFTFNTYA | 119 | IRSQYNNYA | 120 | VRHGNFGNSYVS | 121 |
| StableHu- | | | T | | WFAY | |
| 15 | | | | | | |
| SP34-H2- | GFTFNTYA | 122 | IRSKYHNYA | 123 | VRHGNFGNSYVS | 124 |
| StableHu- | | | T | | WFAY | |
| 16 | | | | | | |
| SP34-H2- | GFTFNTYA | 125 | IRSKVNNYA | 126 | VRHGNFGNSYVS | 127 |
| StableHu- | | | T | | WFAY | |
| 17 | | | | | | |
| SP34-H2- | GFTFNTYA | 128 | IRSSNNNYA | 129 | VRHGNFGNSYVS | 130 |
| StableHu- | | | T | | WFAY | |
| 18 | | | | | | |
| SP34-H2- | GFTFNTYA | 131 | IRSKYNKYA | 132 | VRHGNFGNSYVS | 133 |
| StableHu- | | | T | | WFAY | |
| 19 | | | | | | |
| SP34-H2- | GFTFNTYA | 134 | VRSKASNYA | 135 | VRHGNFGNSYVS | 136 |
| StableHu- | | | T | | WFAY | |
| 20 | | | | | | |
| SP34-H2- | GFTFNTYA | 137 | IRSKSRNYA | 138 | VRHGNFGNSYVS | 139 |
| StableHu- | | | T | | WFAY | |
| 21 | | | | | | |
| SP34-H3- | GFTFNTYA | 140 | IRSKYNNYA | 141 | VREGGSGNYYNV | 142 |
| StableHu- | | | T | | YFAY | |
| 1 | | | | | | |
| SP34-H3- | GFTFNTYA | 143 | IRSKYNNYA | 144 | VRSGRFGESHVG | 145 |
| StableHu- | | | T | | PFAY | |
| 2 | | | | | | |
| SP34-H3- | GFTFNTYA | 146 | IRSKYNNYA | 147 | ARHGEYSSGWPY | 148 |
| StableHu- | | | T | | YFAY | |
| 3 | | | | | | |
| SP34-H3- | GFTFNTYA | 149 | IRSKYNNYA | 150 | VRHGNFGNVGTS | 151 |
| StableHu- | | | T | | YFAY | |
| 4 | | | | | | |
| SP34-H3- | GFTFNTYA | 152 | IRSKYNNYA | 153 | VREGIGGTSYRG | 154 |
| StableHu- | | | T | | NFAY | |
| 5 | | | | | | |
| SP34-L1- | GFTFNTYA | 155 | IRSKYNNYA | 156 | VRHGNFGNSYVS | 157 |
| StableHu- | | | T | | WFAY | |
| 1 | | | | | | |
| SP34-L1- | GFTFNTYA | 158 | IRSKYNNYA | 159 | VRHGNFGNSYVS | 160 |
| StableHu- | | | T | | WFAY | |
| 2 | | | | | | |
| SP34-L1- | GFTFNTYA | 161 | IRSKYNNYA | 162 | VRHGNFGNSYVS | 163 |
| StableHu- | | | T | | WFAY | |
| 3 | | | | | | |
| SP34-L1- | GFTFNTYA | 164 | IRSKYNNYA | 165 | VRHGNFGNSYVS | 166 |
| StableHu- | | | T | | WFAY | |
| 4 | | | | | | |
| SP34-L1- | GFTFNTYA | 167 | IRSKYNNYA | 168 | VRHGNFGNSYVS | 169 |
| StableHu- | | | T | | WFAY | |
| 5 | | | | | | |
| SP34-L1- | GFTFNTYA | 170 | IRSKYNNYA | 171 | VRHGNFGNSYVS | 172 |
| StableHu- | | | T | | WFAY | |
| 6 | | | | | | |
| SP34-L1- | GFTFNTYA | 173 | IRSKYNNYA | 174 | VRHGNFGNSYVS | 175 |
| StableHu- | | | T | | WFAY | |
| 7 | | | | | | |
| SP34-L1- | GFTFNTYA | 176 | IRSKYNNYA | 177 | VRHGNFGNSYVS | 178 |
| StableHu- | | | T | | WFAY | |
| 8 | | | | | | |
| SP34-L1- | GFTFNTYA | 179 | IRSKYNNYA | 180 | VRHGNFGNSYVS | 181 |
| StableHu- | | | T | | WFAY | |
| 9 | | | | | | |
| SP34-L2- | GFTFNTYA | 182 | IRSKYNNYA | 183 | VRHGNFGNSYVS | 184 |
| StableHu- | | | T | | WFAY | |
| 1 | | | | | | |
| SP34-L2- | GFTFNTYA | 185 | IRSKYNNYA | 186 | VRHGNFGNSYVS | 187 |
| StableHu- | | | T | | WFAY | |
| 2 | | | | | | |
| SP34-L2- | GFTFNTYA | 188 | IRSKYNNYA | 189 | VRHGNFGNSYVS | 190 |
| StableHu- | | | T | | WFAY | |
| 3 | | | | | | |
| SP34-L2- | GFTFNTYA | 191 | IRSKYNNYA | 192 | VRHGNFGNSYVS | 193 |
| StableHu- | | | T | | WFAY | |
| 4 | | | | | | |
| SP34-L3- | GFTFNTYA | 194 | IRSKYNNYA | 195 | VRHGNFGNSYVS | 196 |
| StableHu- | | | T | | WFAY | |
| 1 | | | | | | |
| SP34-L3- | GFTFNTYA | 197 | IRSKYNNYA | 198 | VRHGNFGNSYVS | 199 |
| StableHu- | | | T | | WFAY | |
| 2 | | | | | | |
| SP34-L3- | GFTFNTYA | 200 | IRSKYNNYA | 201 | VRHGNFGNSYVS | 202 |
| StableHu- | | | T | | WFAY | |
| 3 | | | | | | |
| SP34-L3- | GFTFNTYA | 203 | IRSKYNNYA | 204 | VRHGNFGNSYVS | 205 |
| StableHu- | | | T | | WFAY | |
| 4 | | | | | | |
| SP34-L3- | GFTFNTYA | 206 | IRSKYNNYA | 207 | VRHGNFGNSYVS | 208 |
| StableHu- | | | T | | WFAY | |
| 5 | | | | | | |
| SP34-L3- | GFTFNTYA | 209 | IRSKYNNYA | 210 | VRHGNFGNSYVS | 211 |
| StableHu- | | | T | | WFAY | |
| 6 | | | | | | |
| SP34-L3- | GFTFNTYA | 212 | IRSKYNNYA | 213 | VRHGNFGNSYVS | 214 |
| StableHu- | | | T | | WFAY | |
| 7 | | | | | | |
| SP34-L3- | GFTFNTYA | 215 | IRSKYNNYA | 216 | VRHGNFGNSYVS | 217 |
| StableHu- | | | T | | WFAY | |
| 8 | | | | | | |
| SP34-L3- | GFTFNTYA | 218 | IRSKYNNYA | 219 | VRHGNFGNSYVS | 220 |
| StableHu- | | | T | | WFAY | |
| 9 | | | | | | |
| SP34-L3- | GFTFNTYA | 221 | IRSKYNNYA | 222 | VRHGNFGNSYVS | 223 |
| StableHu- | | | T | | WFAY | |
| 10 | | | | | | |
| SP34-L3- | GFTFNTYA | 224 | IRSKYNNYA | 225 | VRHGNFGNSYVS | 226 |
| StableHu- | | | T | | WFAY | |
| 11 | | | | | | |
| SP34-L3- | GFTFNTYA | 227 | IRSKYNNYA | 228 | VRHGNFGNSYVS | 229 |
| StableHu- | | | T | | WFAY | |
| 12 | | | | | | |
| SP34-L3- | GFTFNTYA | 230 | IRSKYNNYA | 231 | VRHGNFGNSYVS | 232 |
| StableHu- | | | T | | WFAY | |
| 13 | | | | | | |
| SP34-L3- | GFTFNTYA | 233 | IRSKYNNYA | 234 | VRHGNFGNSYVS | 235 |
| StableHu- | | | T | | WFAY | |
| 14 | | | | | | |
| SP34-L3- | GFTFNTYA | 236 | IRSKYNNYA | 237 | VRHGNFGNSYVS | 238 |
| StableHu- | | | T | | WFAY | |
| 15 | | | | | | |
| SP34-L3- | GFTFNTYA | 239 | IRSKYNNYA | 240 | VRHGNFGNSYVS | 241 |
| StableHu- | | | T | | WFAY | |
| 16 | | | | | | |
| SP34-L3- | GFTFNTYA | 242 | IRSKYNNYA | 243 | VRHGNFGNSYVS | 244 |
| StableHu- | | | T | | WFAY | |
| 17 | | | | | | |
| SP34-L3- | GFTFNTYA | 245 | IRSKYNNYA | 246 | VRHGNFGNSYVS | 247 |
| StableHu- | | | T | | WFAY | |
| 18 | | | | | | |
| SP34-L3- | GFTFNTYA | 248 | IRSKYNNYA | 249 | VRHGNFGNSYVS | 250 |
| StableHu- | | | T | | WFAY | |
| 19 | | | | | | |
| SP34-L3- | GFTFNTYA | 251 | IRSKYNNYA | 252 | VRHGNFGNSYVS | 253 |
| StableHu- | | | T | | WFAY | |
| 20 | | | | | | |
| SP34-L3- | GFTFNTYA | 254 | IRSKYNNYA | 255 | VRHGNFGNSYVS | 256 |
| StableHu- | | | T | | WFAY | |
| 21 | | | | | | |
| SP34-L3- | GFTFNTYA | 257 | IRSKYNNYA | 258 | VRHGNFGNSYVS | 259 |
| StableHu- | | | T | | WFAY | |
| 22 | | | | | | |
| SP34-L3- | GFTFNTYA | 260 | IRSKYNNYA | 261 | VRHGNFGNSYVS | 262 |
| StableHu- | | | T | | WFAY | |
| 23 | | | | | | |
| SP34-L3- | GFTFNTYA | 263 | IRSKYNNYA | 264 | VRHGNFGNSYVS | 265 |
| StableHu- | | | T | | WFAY | |
| 24 | | | | | | |
| SP34-L3- | GFTFNTYA | 266 | IRSKYNNYA | 267 | VRHGNFGNSYVS | 268 |
| StableHu- | | | T | | WFAY | |
| 25 | | | | | | |
| SP34-L3- | GFTFNTYA | 269 | IRSKYNNYA | 270 | VRHGNFGNSYVS | 271 |
| StableHu- | | | T | | WFAY | |
| 26 | | | | | | |
| SP34-L3- | GFTFNTYA | 272 | IRSKYNNYA | 273 | VRHGNFGNSYVS | 274 |
| StableHu- | | | T | | WFAY | |
| 27 | | | | | | |
| SP34-L3- | GFTFNTYA | 275 | IRSKYNNYA | 276 | VRHGNFGNSYVS | 277 |
| StableHu- | | | T | | WFAY | |
| 28 | | | | | | |
| SP34-L3- | GFTFNTYA | 278 | IRSKYNNYA | 279 | VRHGNFGNSYVS | 280 |
| StableHu- | | | T | | WFAY | |
| 29 | | | | | | |
| S34_6- | GFDFNAYA | 281 | IRSKYNNYA | 282 | VRHGNFGNSYVS | 283 |
| F05 | | | T | | WFAY | |

TABLE 8
Anti-CD3-Light Chain CDRs

| | | SEQ ID | | SEQ ID | | SEQ ID |
|---|---|---|---|---|---|---|
| Clone ID | LCDR1 | NO: | LCDR2 | NO: | LCDR3 | NO: |
| SP34-H1- | TGAVTT | 284 | GTN | 285 | ALWYSNLW | 286 |
| StableHu-1 | SNY | | | | V | |
| SP34-H1- | TGAVTT | 287 | GTN | 288 | ALWYSNLW | 289 |
| StableHu-2 | SNY | | | | V | |
| SP34-H1- | TGAVTT | 290 | GTN | 291 | ALWYSNLW | 292 |
| StableHu-3 | SNY | | | | V | |
| SP34-H1- | TGAVTT | 293 | GTN | 294 | ALWYSNLW | 295 |
| StableHu-4 | SNY | | | | V | |
| SP34-H1- | TGAVTT | 296 | GTN | 297 | ALWYSNLW | 298 |
| StableHu-5 | SNY | | | | V | |
| SP34-H1- | TGAVTT | 299 | GTN | 300 | ALWYSNLW | 301 |
| StableHu-6 | SNY | | | | V | |
| SP34-H1- | TGAVTT | 302 | GTN | 303 | ALWYSNLW | 304 |
| StableHu-7 | SNY | | | | V | |
| SP34-H1- | TGAVTT | 305 | GTN | 306 | ALWYSNLW | 307 |
| StableHu-8 | SNY | | | | V | |
| SP34-H1- | TGAVTT | 308 | GTN | 309 | ALWYSNLW | 310 |
| StableHu-9 | SNY | | | | V | |
| SP34-H1- | TGAVTT | 311 | GTN | 312 | ALWYSNLW | 313 |
| StableHu-10 | SNY | | | | V | |
| SP34-H1- | TGAVTT | 314 | GTN | 315 | ALWYSNLW | 316 |
| StableHu-11 | SNY | | | | V | |
| SP34-H1- | TGAVTT | 317 | GTN | 318 | ALWYSNLW | 319 |
| StableHu-12 | SNY | | | | V | |
| SP34-H1- | TGAVTT | 320 | GTN | 321 | ALWYSNLW | 322 |
| StableHu-13 | SNY | | | | V | |
| SP34-H1- | TGAVTT | 323 | GTN | 324 | ALWYSNLW | 325 |
| StableHu-14 | SNY | | | | V | |
| SP34-H1- | TGAVTT | 326 | GTN | 327 | ALWYSNLW | 328 |
| StableHu-15 | SNY | | | | V | |
| SP34-H1- | TGAVTT | 329 | GTN | 330 | ALWYSNLW | 331 |
| StableHu-16 | SNY | | | | V | |
| SP34-H2- | TGAVTT | 332 | GTN | 333 | ALWYSNLW | 334 |
| StableHu-1 | SNY | | | | V | |
| SP34-H2- | TGAVTT | 335 | GTN | 336 | ALWYSNLW | 337 |
| StableHu-2 | SNY | | | | V | |
| SP34-H2- | TGAVTT | 338 | GTN | 339 | ALWYSNLW | 340 |
| StableHu-3 | SNY | | | | V | |
| SP34-H2- | TGAVTT | 341 | GTN | 342 | ALWYSNLW | 343 |
| StableHu-4 | SNY | | | | V | |
| SP34-H2- | TGAVTT | 344 | GTN | 345 | ALWYSNLW | 346 |
| StableHu-5 | SNY | | | | V | |
| SP34-H2- | TGAVTT | 347 | GTN | 348 | ALWYSNLW | 349 |
| StableHu-6 | SNY | | | | V | |
| SP34-H2- | TGAVTT | 350 | GTN | 351 | ALWYSNLW | 352 |
| StableHu-7 | SNY | | | | V | |
| SP34-H2- | TGAVTT | 353 | GTN | 354 | ALWYSNLW | 355 |
| StableHu-8 | SNY | | | | V | |
| SP34-H2- | TGAVTT | 356 | GTN | 357 | ALWYSNLW | 358 |
| StableHu-9 | SNY | | | | V | |
| SP34-H2- | TGAVTT | 359 | GTN | 360 | ALWYSNLW | 361 |
| StableHu-10 | SNY | | | | V | |
| SP34-H2- | TGAVTT | 362 | GTN | 363 | ALWYSNLW | 364 |
| StableHu-11 | SNY | | | | V | |
| SP34-H2- | TGAVTT | 365 | GTN | 366 | ALWYSNLW | 367 |
| StableHu-12 | SNY | | | | V | |
| SP34-H2- | TGAVTT | 368 | GTN | 369 | ALWYSNLW | 370 |
| StableHu-13 | SNY | | | | V | |
| SP34-H2- | TGAVTT | 371 | GTN | 372 | ALWYSNLW | 373 |
| StableHu-14 | SNY | | | | V | |
| SP34-H2- | TGAVTT | 374 | GTN | 375 | ALWYSNLW | 376 |
| StableHu-15 | SNY | | | | V | |
| SP34-H2- | TGAVTT | 377 | GTN | 378 | ALWYSNLW | 379 |
| StableHu-16 | SNY | | | | V | |
| SP34-H2- | TGAVTT | 380 | GTN | 381 | ALWYSNLW | 382 |
| StableHu-17 | SNY | | | | V | |
| SP34-H2- | TGAVTT | 383 | GTN | 384 | ALWYSNLW | 385 |
| StableHu-18 | SNY | | | | V | |
| SP34-H2- | TGAVTT | 386 | GTN | 387 | ALWYSNLW | 388 |
| StableHu-19 | SNY | | | | V | |
| SP34-H2- | TGAVTT | 389 | GTN | 390 | ALWYSNLW | 391 |
| StableHu-20 | SNY | | | | V | |
| SP34-H2- | TGAVTT | 392 | GTN | 393 | ALWYSNLW | 394 |
| StableHu-21 | SNY | | | | V | |
| SP34-H3- | TGAVTT | 395 | GTN | 396 | ALWYSNLW | 397 |
| StableHu-1 | SNY | | | | V | |
| SP34-H3- | TGAVTT | 398 | GTN | 399 | ALWYSNLW | 400 |
| StableHu-2 | SNY | | | | V | |
| SP34-H3- | TGAVTT | 401 | GTN | 402 | ALWYSNLW | 403 |
| StableHu-3 | SNY | | | | V | |
| SP34-H3- | TGAVTT | 404 | GTN | 405 | ALWYSNLW | 406 |
| StableHu-4 | SNY | | | | V | |
| SP34-H3- | TGAVTT | 407 | GTN | 408 | ALWYSNLW | 409 |
| StableHu-5 | SNY | | | | V | |
| SP34-L1- | TGAVTT | 410 | GTN | 411 | ALWYSNLW | 412 |
| StableHu-1 | RNY | | | | V | |
| SP34-L1- | TGAVTT | 413 | GTN | 414 | ALWYSNLW | 415 |
| StableHu-2 | STY | | | | V | |
| SP34-L1- | TGAVS | 416 | GTN | 417 | ALWYSNLW | 418 |
| StableHu-3 | TNYY | | | | V | |
| SP34-L1- | TGAVP | 419 | GTN | 420 | ALWYSNLW | 421 |
| StableHu-4 | TSNY | | | | V | |
| SP34-L1- | TGAVP | 422 | GTN | 423 | ALWYSNLW | 424 |
| StableHu-5 | TSLY | | | | V | |
| SP34-L1- | TGAVT | 425 | GTN | 426 | ALWYSNLW | 427 |
| StableHu-6 | PSNY | | | | V | |
| SP34-L1- | GGSVT | 428 | GTN | 429 | ALWYSNLW | 430 |
| StableHu-7 | THNY | | | | V | |
| SP34-L1- | TGAVT | 431 | GTN | 432 | ALWYSNLW | 433 |
| StableHu-8 | SPSY | | | | V | |
| SP34-L1- | LGSVTT | 434 | GTN | 435 | ALWYSNLW | 436 |
| StableHu-9 | FNY | | | | V | |
| SP34-L2- | TGAVTT | 437 | GTL | 438 | ALWYSNLW | 439 |
| StableHu-1 | SNY | | | | V | |
| SP34-L2- | TGAVTT | 440 | AYN | 441 | ALWYSNLW | 442 |
| StableHu-2 | SNY | | | | V | |
| SP34-L2- | TGAVTT | 443 | GQN | 444 | ALWYSNLW | 445 |
| StableHu-3 | SNY | | | | V | |
| SP34-L2- | TGAVTT | 446 | STD | 447 | ALWYSNLW | 448 |
| StableHu-4 | SNY | | | | V | |
| SP34-L3- | TGAVTT | 449 | GTN | 450 | ALYYSFSW | 451 |
| StableHu-1 | SNY | | | | V | |
| SP34-L3- | TGAVTT | 452 | GTN | 453 | QTWGNNIW | 454 |
| StableHu-2 | SNY | | | | V | |
| SP34-L3- | TGAVTT | 455 | GTN | 456 | AIWYSRVW | 457 |
| StableHu-3 | SNY | | | | V | |
| SP34-L3- | TGAVTT | 458 | GTN | 459 | AIWYSTSW | 460 |
| StableHu-4 | SNY | | | | V | |
| SP34-L3- | TGAVTT | 461 | GTN | 462 | VIWHSGAW | 463 |
| StableHu-5 | SNY | | | | V | |
| SP34-L3- | TGAVTT | 464 | GTN | 465 | WVWHSSLW | 466 |
| StableHu-6 | SNY | | | | L | |
| SP34-L3- | TGAVTT | 467 | GTN | 468 | LLSYSRSW | 469 |
| StableHu-7 | SNY | | | | V | |
| SP34-L3- | TGAVTT | 470 | GTN | 471 | LLFYNRLW | 472 |
| StableHu-8 | SNY | | | | V | |
| SP34-L3- | TGAVTT | 473 | GTN | 474 | VIWHGSVW | 475 |
| StableHu-9 | SNY | | | | V | |
| SP34-L3- | TGAVTT | 476 | GTN | 477 | ANWKSNIL | 478 |
| StableHu-10 | SNY | | | | V | |
| SP34-L3- | TGAVTT | 479 | GTN | 480 | RIWHSSSW | 481 |
| StableHu-11 | SNY | | | | V | |
| SP34-L3- | TGAVTT | 482 | GTN | 483 | RLDYSGVW | 484 |
| StableHu-12 | SNY | | | | V | |
| SP34-L3- | TGAVTT | 485 | GTN | 486 | VIWHNLAW | 487 |
| StableHu-13 | SNY | | | | V | |
| SP34-L3- | TGAVTT | 488 | GTN | 489 | TIWHSGVW | 490 |
| StableHu-14 | SNY | | | | V | |
| SP34-L3- | TGAVTT | 491 | GTN | 492 | AIWPGDAW | 493 |
| StableHu-15 | SNY | | | | V | |
| SP34-L3- | TGAVTT | 494 | GTN | 495 | LTWHTSAW | 496 |
| StableHu-16 | SNY | | | | V | |
| SP34-L3- | TGAVTT | 497 | GTN | 498 | QAWYSNIG | 499 |
| StableHu-17 | SNY | | | | V | |
| SP34-L3- | TGAVTT | 500 | GTN | 501 | AIWYSTAYI | 502 |
| StableHu-18 | SNY | | | | | |
| SP34-L3- | TGAVTT | 503 | GTN | 504 | LIWYNNIWV | 505 |
| StableHu-19 | SNY | | | | | |
| SP34-L3- | TGAVTT | 506 | GTN | 507 | RVWHSDA | 508 |
| StableHu-20 | SNY | | | | WV | |
| SP34-L3- | TGAVTT | 509 | GTN | 510 | LIWYGSAW | 511 |
| StableHu-21 | SNY | | | | V | |
| SP34-L3- | TGAVTT | 512 | GTN | 513 | AAWDVSLW | 514 |
| StableHu-22 | SNY | | | | V | |
| SP34-L3- | TGAVTT | 515 | GTN | 516 | AAWHDNA | 517 |
| StableHu-23 | SNY | | | | WV | |
| SP34-L3- | TGAVTT | 518 | GTN | 519 | QPYYSNNW | 520 |
| StableHu-24 | SNY | | | | V | |
| SP34-L3- | TGAVTT | 521 | GTN | 522 | QAWDRSIW | 523 |
| StableHu-25 | SNY | | | | V | |
| SP34-L3- | TGAVTT | 524 | GTN | 525 | LLYYGSIWV | 526 |
| StableHu-26 | SNY | | | | | |
| SP34-L3- | TGAVTT | 527 | GTN | 528 | AIWYDNIW | 529 |
| StableHu-27 | SNY | | | | V | |
| SP34-L3- | TGAVTT | 530 | GTN | 531 | LSWHSSSW | 532 |
| StableHu-28 | SNY | | | | V | |
| SP34-L3- | TGAVTT | 533 | GTN | 534 | LIWQTSAW | 535 |
| StableHu-29 | SNY | | | | V | |
| S34 6-F05 | TGAVTT | 536 | GTN | 537 | AAWHDNA | 538 |
| | SNY | | | | WV | |

### EMBODIMENTS

Embodiment 1. A method of making an epitope-targeted, conditionally-activated, pro-drug, antibody in a single discovery cycle comprising: obtaining an epitope-specific antibody comprising an antigen binding site with complementarity determining regions (CDRs) from an in vivo, in vitro, or in silico antibody library; concurrently designing or obtaining in silico one or more engineered epitope masks with a linker, wherein the linker comprises a peptide, a polymer, or a chemical-linker that is cleaved in vivo at a target site by an enzyme or cleaved chemically, or a structural-switch linker that switches to an active state at a target site by one or more environmental condition(s), and wherein the one or more engineered epitope masks binds the epitope-specific antibody at the CDRs of the epitope-specific antibody; and making the epitope-targeted, conditionally-activated, pro-drug, antibody as a fusion protein or an antibody with the one or more engineered epitope masks linked to the antibody via the linker, wherein the one or more engineered epitope masks are conditionally bound to the CDRs of the epitope-specific antibody, wherein cleavage of the linker releases the one or more engineered epitope masks from the antigen binding site.

Embodiment 2. The method of embodiment 1, wherein the peptide, polymer, or chemical-linker epitope are not obtained by steric inhibition in vitro.

Embodiment 3. The method of embodiments 1 or 2, wherein the antibody and the peptide, polymer, or chemical-linker epitope are selected concurrently in a single discovery cycle.

Embodiment 4. The method of any one of embodiments 1 to 3, wherein the linker is a cleavable peptide, polymer, or chemical linker.

Embodiment 5. The method of any one of embodiments 1 to 4, wherein the linker is a structure-switch peptide, polymer, or chemical linker.

Embodiment 6. The method of embodiment 4, further comprising concurrently designing in silico the peptide, polymer, or epitope and the cleavable or structure-switch peptide, polymer, or chemical link.

Embodiment 7. The method of any one of embodiments 1 to 6, further comprising designing a cleavable or structure-switch peptide, polymer, or chemical linker to at least one of: increase or decrease proteolytic or cleavage activity of the antibody, one or more engineered-epitopes, the linker, or all three; increase or decrease an on/off tissue conditional-activation of the antibody, one or more engineered-epitopes, the linker, or all three; increase or decrease solubility of the antibody, one or more engineered-epitopes, the linker, or all three; increase or decrease expression of the antibody; increase or decrease stability of the antibody, one or more engineered-epitopes, the linker, or all three; increase or decrease immunogenicity of the antibody, one or more engineered-epitopes, the linker, or all three; mask the complementarity determining regions (CDRs) that generate anti-drug antibodies; add an inhibitory or stimulatory activity in the one or more engineered-epitopes, the linker, or both; or add a cell or tissue localization in the one or more engineered-epitopes, the linker, or both.

Embodiment 8. The method of any one of embodiments 1 to 7, wherein the peptide, polymer, or epitope and the linker are not designed concurrently.

Embodiment 9. The method of any one of embodiments 1 to 8, further comprising modifying the peptide, polymer, or chemical linker of the linker to increase or decrease cleavage of the linker at the target site.

Embodiment 10. The method of any one of embodiments 1 to 9, wherein the one or more engineered epitope masks and the scaffold are not obtained from a random library of peptides, polymers, or chemical linkers.

Embodiment 11. The method of any one of embodiments 1 to 10, wherein the one or more engineered epitope masks and the scaffold are modeled in silico to fit an antigen binding site of a specific antibody.

Embodiment 12. The method of any one of embodiments 1 to 11, wherein the steps of obtaining the epitope-specific antibody comprising and the one or more engineered epitope masks and the scaffold further comprises: training a machine learning model based on an epitope-specific antibody record and one or more engineered epitopes, or representations thereof, and a first plurality of scores, each epitope-specific antibody record from the first plurality of epitope-specific antibody record and one or more engineered epitopes associated with each score from the first plurality of scores; and executing, after the training, the machine learning model to generate a second plurality of epitope-specific antibody records and one or more engineered epitopes having at least one desired score;
the second plurality of epitope-specific antibody records configured to be received as input in computational protein modeling to generate the one or more engineered epitopes based on a second plurality of epitope-specific antibody record with the one or more engineered epitopes in an antigen binding site of the epitope-specific antibody.

Embodiment 13. The method of any one of embodiments 1 to 12, further comprising receiving a representation of the one or more engineered epitope masks and the linker; and generating a first plurality of epitope-specific antibody records from a predetermined portion of the one or more engineered epitope masks and the linker, each epitope-specific antibody records from the first plurality of epitope-specific antibody records comprising target residue positions and linker residue positions, each target residue position corresponding to one target residue from a plurality of target residues of the one or more engineered epitope masks and the linker.

Embodiment 14. The method of embodiment 11, further comprising labeling a first plurality of epitope-specific antibody records by, for each epitope-specific antibody records from the first plurality of epitope-specific antibody records: performing computational protein modeling on that epitope-specific antibody record to generate a polypeptide structure, calculating a score for the polypeptide structure, and associating the score with that epitope-specific antibody record.

Embodiment 15. An epitope-targeted, conditionally-activated, pro-drug, antibody comprising: an antigen binding site with complementarity determining regions (CDRs) from an epitope-specific antibody identified from an in vivo, in vitro, or in silico antibody library; and one or more engineered epitope masks on a linker, wherein the linker comprises a peptide, a polymer, or a chemical-linker that is cleavable in vivo at a target site by an enzyme or cleaved chemically, and wherein the one or more engineered epitope masks binds the epitope-specific antibody at the CDRs of the epitope-specific antibody; and wherein the epitope-targeted, conditionally-activated, pro-drug, antibody is a fusion protein or an antibody with the one or more engineered epitope masks linked to the antibody via the linker, wherein the one or more engineered epitope masks are conditionally bound to the CDRs of the epitope-specific antibody, and wherein cleavage of the linker releases the one or more engineered epitope masks from the antigen binding site from the epitope-targeted, conditionally-activated, pro-drug, antibody.

Embodiment 16. The epitope-targeted, conditionally-activated, pro-drug, antibody of embodiment 15, wherein the antibody comprises:
a heavy chain variable domain (VH) complementarity determining region (CDRs) 1 to CDR3 comprising an amino acid sequence of any one of the following SEQ ID NOS: 1, 2, 3; 15, 16, 17; 29, 30, 31; 32, 33, 34; 35, 36, 37; 38, 39, 40; 41, 42, 43; 44, 45, 46; 47, 48, 49; 50, 51, 52; 53, 54, 55; 56, 57, 58; 59, 60, 61; 62, 63, 64; 65, 66, 67; 68, 69, 70; 71, 72, 73; 74, 75, 76; 77, 78, 79; 80, 81, 82; 83, 84, 85; 86, 87, 88; 89, 90, 91; 92, 93, 94; 95, 96, 97; 98, 99, 100; 101, 102, 103; 104, 105, 106; 107, 108, 109; 110, 111, 112; 113, 114, 115; 116, 117, 118; 119, 120, 121; 122, 123, 124; 125, 126, 127; 128, 129, 130; 131, 132, 133; 134, 135, 136; 137, 138, 139; 140, 141, 142; 143, 144, 145; 146, 147, 148; 149, 150, 151; 152, 153, 154; 155, 156, 157; 158, 159, 160; 161, 162, 163; 164, 165, 166; 167, 168, 169; 170, 171, 172; 173, 174, 175; 176, 177, 178; 179, 180, 181; 182, 183, 184; 185, 186, 187; 188, 189, 190; 191, 192, 193; 194, 195, 196; 197, 198, 199; 200, 201, 202; 203, 204, 205; 206, 207, 208; 209, 210, 211; 212, 213, 214; 215, 216, 217; 218, 219, 220; 221, 222, 223; 224, 225, 226; 227, 228, 229; 230, 231, 232; 233, 234, 235; 236, 237, 238; 239, 240, 241; 242, 243, 244; 245, 246, 247; 248, 249, 250; 251, 252, 253; 254, 255, 256; 257, 258, 259; 260, 261, 262; 263, 264, 265; 266, 267, 268; 269, 270, 271; 272, 273, 274; 275, 276, 277; 278, 279, 280; or 281, 282, 283;
a light chain variable domain (VL) CDR1 to CDR3 comprising the amino acid sequence of any one of the following SEQ ID NOs: 4, 5, 6; 18, 19, 20; 284, 285, 286; 287, 288, 289; 290, 291, 292; 293, 294, 295; 296, 297, 298; 299, 300, 301; 302, 303, 304; 305, 306, 307; 308, 309, 310; 311, 312, 313; 314, 315, 316; 317, 318, 319; 320, 321, 322; 323, 324, 325; 326, 327, 328; 329, 330, 331; 332, 333, 334; 335, 336, 337; 338, 339, 340; 341, 342, 343; 344, 345, 346; 347, 348, 349; 350, 351, 352; 353, 354, 355; 356, 357, 358; 359, 360, 361; 362, 363, 364; 365, 366, 367; 368, 369, 370; 371, 372, 373; 374, 375, 376; 377, 378, 379; 380, 381, 382; 383, 384, 385; 386, 387, 388; 389, 390, 391; 392, 393, 394; 395, 396, 397; 398, 399, 400; 401, 402, 403; 404, 405, 406; 407, 408, 409; 410, 411, 412; 413, 414, 415; 416, 417, 418; 419, 420, 421; 422, 423, 424; 425, 426, 427; 428, 429, 430; 431, 432, 433; 434, 435, 436; 437, 438, 439; 440, 441, 442; 443, 444, 445; 446, 447, 448; 449, 450, 451; 452, 453, 454; 455, 456, 457; 458, 459, 460; 461, 462, 463; 464, 465, 466; 467, 468, 469; 470, 471, 472; 473, 474, 475; 476, 477, 478; 479, 480, 481; 482, 483, 484; 485, 486, 487; 488, 489, 490; 491, 492, 493; 494, 495, 496; 497, 498, 499; 500, 501, 502; 503, 504, 505; 506, 507, 508; 509, 510, 511; 512, 513, 514; 515, 516, 517; 518, 519, 520; 521, 522, 523; 524, 525, 526; 527, 528, 529; 530, 531, 532; 533, 534, 535; or 536, 537, 538.

Embodiment 17. The epitope-targeted, conditionally-activated, pro-drug, antibody of embodiment 15 or 16, wherein the antibody comprises a VH and VL pair comprising the amino acid sequence of any one of the following SEQ ID NOS: 7 and 8, 9 and 10, 21 and 22, or 23 and 24.

Embodiment 18. The epitope-targeted, conditionally-activated, pro-drug, antibody of any one of embodiments 15 to 17, wherein the antibody is a monoclonal antibody.

Embodiment 19. The epitope-targeted, conditionally-activated, pro-drug, antibody of any one of embodiments 15 to 18, wherein the antibody is a full-length antibody.

Embodiment 20. The epitope-targeted, conditionally-activated, pro-drug, antibody of any one of embodiments 15 to 19, wherein the antibody is an antibody fragment.

Embodiment 21. The epitope-targeted, conditionally-activated, pro-drug, antibody of embodiments 18 or 19, wherein the antibody is fused to an Fc domain of any one of the following: human IgG1, human IgG2, human IgG3, and human IgG4.

Embodiment 22. The epitope-targeted, conditionally-activated, pro-drug, antibody of any one of embodiments 15 to 21, wherein the antibody specifically binds to: 4-1BBL, AFP, AKAP-4, ALK, androgen receptor, bcr-abl, Clorf186, CA-125, CA6, CA6, CA19-9, CAMPATH-1, Carbonic anhydrase IX, carcinoembryonic antigen (CEA), CCR8, CD1, CD2, CD3, CD4, CD5, CD8, CD16A, CD19, CD1A, CD20, CD204, CD204, CD204, CD206, CD206, CD206, CD25, CD276, CD28, CD30, CD301, CD301, CD32A, CD32B, CD33, CD36, CD37, CD39, CD40, CD45, CD47, CD5, CD64, CD73, CEA, CLEC4C, CLDN16, CLDN6, CLDN18.2, CLEC10A, CLEC12A, CLECSA, CLEC9A, CMET, CTCFL, cyclin B1, CYP1B1, DC-SIGN, DEC-205, Dectin1, Dectin2, DLL3, EGFR, EGFRvIII, Endoglin, endosialin, EPCAM, EphA2, epidermal growth factor, ERG, ETV6-AML, ferritin, fibroblast activation protein (FAP), FLT3, folate-binding protein, Fos-related antigen 1, FRA, fucosyl GM1, G250, GD2, GD3, Glycoprotein A33, GloboH, GLP-3, GM2, GM3, gp100, HER2, HER2/neu, HER3, HLD-DR, HMWMAA, HPV E6, HPV E7, hTERT, HVEM, IL-2 receptor, IV1, Latent-TGFB, LCK, Legumain, Ley, LIV1, LMP2, LY6E, MAD-CT-1, MAD-CT-2, MAGE A1, MAGE A3, mannose scavenger receptor1, MARCO, MelanA/MART1, mesothelin (MSLN), metalloproteinase, ML-IAP, MSLN, MUC1, MUC15, MUC16, MYCN, NA17, NAPI2B, NAPI2B, NY-BR-1, NY-ESO-1, OX40L, OY-TES1, p185HER2, P53 mutant, P53 nonmutant, PAGE4, PAP, PAX3, PAX5, PDGFR-B, PDL, PDL1, PLAV1, polysialic acid, PR1, PSA, PSCA, PSMA, PTK7, Ras mutant, RGS5, RhoC, RON, ROR1, ROR2, RRC15, SART3, SIRPA, Sperm protein 17, SSX2, STn, survivin, TAG-72, tenascin, TGFB, Tie 3, TMEM238, TMPRSS3, TMPRSS4, Tn, TRA6, TROP2, TRP-2, tyrosinase, UPK1B, vascular endothelial growth factor, VEGFR, VEGFR2, VISTA, VTCN1, WT1, XAGE 1, or Y6E.

Embodiment 23. The epitope-targeted, conditionally-activated, pro-drug, antibody of any one of embodiments 15 to 22, wherein the antibody is ABT806 (EGFRvIII), adecatumumab (EPCAM), alemtuzumab (CD33), AMG595 (EGFRvIII), anetumab (MSLN), anti-ACE2, anti-EphA2 (EphA2), anti-hyaluronidase, anti-neuraminidase, anti-NY-ESO-1, anti-PTK7 (PTK7), cetuximab (EGFR), cirmtuzumab (ROR1), clivatuzumab (MUC1), CT-011 (PD1), DS-8895a variant 1 (EphA2), DS-8895a variant 2 (EphA2), durvalumab (PDL1) anti-MAGE-A3, edrecolomab (EPCAM), farletuzumab (FRA/folate receptor alpha), Gemtuzumab ozogamicin (CD33), huDS6 (CA6), humanized Ab 2-3 (CEA), humanized PR1A3 (CEA), ibritumomab tiuxetan (CD52), IMAB362/claudiximab (Claudin18.2), ipilimumab (CTLA4), J591 variant 1 (PSMA), J591 variant 2 (PSMA), ladiratuzumab (LIV1), lifastuzumab (NAPI2B), MEDI-547 (EphA2), mirvetuximab (FRA), narnatumab (RON), nimotuzumab (EGFR), nivolumab (PD1), onartuzumab (c-MET), panitumumab (EGFR), patritumab (HER3), pembrolizumab (PD1), pertuzumab (HER2/neu), PF-06647020 (PTK7), RG7841 (LY6E), rituximab (CD20), rovalpituzumab (DLL3), sacituzumab (TROP2), sibrotuzumab (FAP), sofituzumab (MUC16), tositumomab (CD20), trastuzumab (HER2/neu), tremelimumab (CP-675,206)(CTLA4), or zalutumumab (EGFR).

Embodiment 24. The epitope-targeted, conditionally-activated, pro-drug, antibody of any one of embodiments 15 to 23, wherein the linker is a peptide cleaved by actinidain, activated protein C, ADAM10, ADAM12, ADAMS, bromelain, bromelain, calpain, Caspase, caspase-3, Cathepsin, Cathepsin B, Cathepsin C, Cathepsin D, Cathepsin E, cathepsin G, Cathepsin K, Cathepsin L, Chymase, chymosin, chymotrypsin-like protease, CMV protease, collagenase, DESC1, dipeptidyl peptidase, dipeptidyl peptidase IV (DPPIV/CD26), disintegrin and metalloproteinase (ADAM), DPP-4, Elastase, elastase-like protease, enterokinase, Factor Xa, FAP, FAP (FAP-α), Granzyme B, Guanidinobenzoatase, Hepsin, HIV-1 protease, hK1, hK15, hK3, HSV protease, HtrAl, HumNeutrophil Elastase, interleukin-13 converting enzyme, kallikrein, kallikrein-related peptidase (KLK), Lactoferrin, legumain, Marapsin, mast cell chymase, mast cell tryptase, matriptase, Matriptase-2, matrix metalloprotease (MMP), metalloendopeptidase, metalloexopeptidase, Mirl-CP, metalloproteases (MMP1, MMP2, MMP3, MMP7, MMP8, MMP9, MMP10, MMP11, MMP12, MMP13, MMP14), nepenthesin, neutrophil elastase, neutrophil serine protease 4, NS3/4A, PACE4, papain, pepsin, plasmepsin, plasmin, prostate-specific antigen (PSA), proteinase 3, renin, secretase, stromelysin, subtilisin-like protease, thrombin, tissue plasminogen activator (tPA), transmembrane Serine Protease (TMPRSS), trypsin, trypsin-like protease, tryptase, type II transmembrane serine protease (TTSP), Type IV collagenase, urokinase plasminogen activator (uPA), or urokinase plasminogen activator receptor (uPAR).

Embodiment 25. The epitope-targeted, conditionally-activated, pro-drug, antibody of any one of embodiments 15 to 24, wherein the linker comprises non-amino acid cleavage polymers or linkers selected from nucleic acids, lipids, carbohydrates, or chemical linkers, such as those that are cleaved or dissociated by radiation, electromagnetic, pH, chemically, or enzymatically.

Embodiment 26. The epitope-targeted, conditionally-activated, pro-drug, antibody of any one of embodiments 15 to 25, wherein the linker comprises a structure-switch linker that includes amino acid or non-amino acid polymers that adopt an inactivated prodrug conformation with the mask blocking the antibody-antigen binding site and upon exposure to specific microenvironmental conditions the linker switches to an activated antibody conformation.

Embodiment 27. The epitope-targeted, conditionally-activated, pro-drug, antibody of embodiment 26, wherein the microenvironmental conditions are selected from pH, enzyme(s), or metabolite(s).

Embodiment 28. A method of treating a disease in a subject in need thereof, comprising administering to the subject a therapeutically effective amount of the epitope-targeted, conditionally-activated, pro-drug, antibody of any one of embodiments 15 to 27.

Embodiment 29. The method of embodiment 28, where the disease is an autoimmune disease, an inflammatory disease, an infectious disease, or a cancer.

Embodiment 30. The method of embodiments 28 or 29, wherein the cancer is selected from: acute lymphoblastic leukemia, acute myelogenous leukemia, adrenal glands cancer, bladder cancer, bone marrow cancer, breast cancer, chronic lymphocytic leukemia, chronic myeloid leukemia, central nervous system cancer, colorectal cancer, colorectal carcinoma, endometrial cancer, endometrial carcinoma, gastric cancer, gut carcinoma, head and neck squamous cell carcinoma, hematopoietic malignancies, liver cancer, lung carcinoma, lymph node cancer, melanoma, metastatic colorectal cancer, ovarian cancer, pancreatic adenocarcinoma, pituitary tumor, prostate cancer, prostate carcinoma, renal cell carcinoma, retinal cancer, sarcoma, skin cancer, spleen cancer, stomach cancer, thymus cancer, or thyroid cancer.

Embodiment 31. The method of any one of embodiments 28 to 30, wherein the subject is human.

Embodiment 32. A nucleic acid encoding an epitope-targeted, conditionally-activated, pro-drug, antibody comprising:
a nucleic acid encoding an antigen binding site with complementarity determining regions (CDRs) from an antibody identified from an in vivo, in vitro, or in silico antibody library; and
one or more engineered epitope masks connected to a linker, wherein the linker comprises a peptide, a polymer, or a chemical-linker that is cleavable in vivo at a target site by an enzyme or cleaved chemically, and wherein the one or more engineered epitope masks binds the epitope-specific antibody at the CDRs of the epitope-targeted antibody; and
wherein the epitope-targeted, conditionally-activated, pro-drug, antibody is a fusion protein or an antibody with the one or more engineered epitope masks linked to the antibody via the linker,
wherein the one or more engineered epitope masks are conditionally bound to the CDRs of the epitope-specific antibody, and
wherein cleavage of the linker releases the one or more engineered epitope masks from the antigen binding site.

Embodiment 33. The nucleic acid of embodiment 32, the nucleic acid comprises:
a first polynucleotide encoding a heavy chain variable domain having at least 95, 96, 97, 98, 99, or 100% sequence identity to SEQ ID NOS: 11, 13, 25, 27; and
a second polynucleotide encoding light chain variable domain encoding polynucleotide having at least 95, 96, 97, 98, 99, or 100% sequence identity to SEQ ID NOS: 12, 14, 26, 28.

Embodiment 34. The nucleic acid of embodiment 33, wherein the antibody is a monoclonal, bispecific, multivalent, multi-specific, diabody, chimeric, scFv antibody, or fragments thereof.

Embodiment 35. The nucleic acid of embodiments 33 or 34, wherein an antibody binding domain is fused to an Fc domain of any one of the following: human IgG1, human IgG2, human IgG3, and human IgG4.

Embodiment 36. The nucleic acid of any one of embodiments 32 to 35, wherein the nucleic acid sequence is optimized for expression in a bacterial, fungal, mammalian, insect, or plant cell.

Embodiment 37. A vector comprising the nucleic acid of embodiment 32.

Embodiment 38. A host cell comprising nucleic acid the vector of embodiment 37.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/of” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of making an epitope-targeted, conditionally-activated, pro-drug, antibody in a single discovery cycle comprising:
obtaining an epitope-specific antibody comprising an antigen binding site with complementarity determining regions (CDRs) from an in vivo, in vitro, or in silico antibody library;
concurrently designing or obtaining in silico one or more engineered epitope masks connected to a linker, wherein the linker comprises a peptide, a polymer, or a chemical-linker that is cleaved in vivo at a target site by an enzyme or cleaved chemically, or a structural-switch linker that switches to an active state at the target site by one or more environmental condition(s), and wherein the one or more engineered epitope masks binds the epitope-specific antibody at the CDRs of the epitope-specific antibody; and
making the epitope-targeted, conditionally-activated, pro-drug, antibody as a fusion protein or an antibody with the one or more engineered epitope masks linked to the antibody via the linker, wherein the one or more engineered epitope masks are conditionally bound to the CDRs of the epitope-specific antibody,
wherein cleavage of the linker releases the one or more engineered epitope masks from the antigen binding site.

2. The method of claim 1, wherein the peptide, polymer, or chemical-linker, the epitope, or both are not obtained by stearic inhibition in vitro.

3. The method of claim 1, wherein the antibody and the peptide, polymer, or chemical-linker epitope are selected concurrently in a single discovery cycle.

4. The method of claim 1, wherein the linker is a cleavable peptide, polymer, or chemical linker.

5. The method of claim 1, wherein the linker is a structure-switch peptide, polymer, or chemical linker.

6. The method of claim 4, further comprising concurrently designing in silico the peptide, polymer, or epitope and a cleavable or structure-switch peptide, polymer, or chemical link.

7. The method of claim 1, further comprising designing a cleavable or structure-switch peptide, polymer, or chemical linker to at least one of:
increase or decrease proteolytic or cleavage activity of the antibody, one or more engineered-epitopes, the linker, or all three;
increase or decrease an on/off tissue conditional-activation of the antibody, one or more engineered-epitopes, the linker, or all three;
increase or decrease solubility of the antibody, one or more engineered-epitopes, the linker, or all three;
increase or decrease expression of the antibody;
increase or decrease stability of the antibody, one or more engineered-epitopes, the linker, or all three;
increase or decrease immunogenicity of the antibody, one or more engineered-epitopes, the linker, or all three;
mask the complementarity determining regions (CDRs) that generate anti-drug antibodies;
add an inhibitory or stimulatory activity in the one or more engineered-epitopes, the linker, or both; or
add a cell or tissue localization in the one or more engineered-epitopes, the linker, or both.

8. The method of claim 1, wherein the peptide, polymer, or epitope and the linker are not designed concurrently.

9. The method of claim 1, further comprising modifying the peptide, polymer, or chemical linker of the linker to increase or decrease cleavage of the linker at the target site.

10. The method of claim 1, wherein the one or more engineered epitope masks and the linker are not obtained from a random library of peptides, polymers, or chemical linkers.

11. The method of claim 1, wherein the one or more engineered epitope masks and the linker are modeled in silico to fit an antigen binding site of a specific antibody.

12. The method of claim 1, wherein the steps of obtaining the epitope-specific antibody comprising and the one or more engineered epitope masks and the linker further comprises:
training a machine learning model based on an epitope-specific antibody record and one or more engineered epitopes, or representations thereof, and a first plurality of scores, each epitope-specific antibody record from the first plurality of epitope-specific antibody record and one or more engineered epitopes associated with each score from the first plurality of scores; and
executing, after the training, the machine learning model to generate a second plurality of epitope-specific antibody records and one or more engineered epitopes having at least one desired score;
the second plurality of epitope-specific antibody records configured to be received as input in computational protein modeling to generate the one or more engineered epitopes based on a second plurality of epitope-specific antibody record with the one or more engineered epitopes in an antigen binding site of the epitope-specific antibody.

13. The method of claim 1, further comprising receiving a representation of the one or more engineered epitope masks and the linker; and
generating a first plurality of epitope-specific antibody records from a predetermined portion of the one or more engineered epitope masks and the linker, each epitope-specific antibody records from the first plurality of epitope-specific antibody records comprising target residue positions and linker residue positions, each target residue position corresponding to one target residue from a plurality of target residues of the one or more engineered epitope masks and the linker.

14. The method of claim 1, further comprising labeling a first plurality of epitope-specific antibody records by, for each epitope-specific antibody records from the first plurality of epitope-specific antibody records:
performing computational protein modeling on that epitope-specific antibody record to generate a polypeptide structure,
calculating a score for the polypeptide structure, and
associating the score with that epitope-specific antibody record.

15. An epitope-targeted, conditionally-activated, pro-drug, antibody comprising:
an antigen binding site with complementarity determining regions (CDRs) from an epitope-specific antibody identified from an in vivo, in vitro, or in silico antibody library; and
one or more engineered epitope masks connected to a linker, wherein the linker comprises a peptide, a polymer, or a chemical-linker that is cleavable in vivo at a target site by an enzyme or cleaved chemically, and wherein the one or more engineered epitope masks binds the epitope-specific antibody at the CDRs of the epitope-specific antibody; and
wherein the epitope-targeted, conditionally-activated, pro-drug, antibody is a fusion protein or an antibody with the one or more engineered epitope masks linked to the antibody via the linker,
wherein the one or more engineered epitope masks are conditionally bound to the CDRs of the epitope-specific antibody, and
wherein cleavage of the linker releases the one or more engineered epitope masks from the antigen binding site from the epitope-targeted, conditionally-activated, pro-drug, antibody.

16. The epitope-targeted, conditionally-activated, pro-drug, antibody of claim 15, wherein the antibody comprises:
a heavy chain variable domain (VH) complementarity determining region (CDRs) 1 to CDR3 comprising an amino acid sequence of any one of SEQ ID NOS: 1, 2, 3; 15, 16, 17; 29, 30, 31; 32, 33, 34; 35, 36, 37; 38, 39, 40; 41, 42, 43; 44, 45, 46; 47, 48, 49; 50, 51, 52; 53, 54, 55; 56, 57, 58; 59, 60, 61; 62, 63, 64; 65, 66, 67; 68, 69, 70; 71, 72, 73; 74, 75, 76; 77, 78, 79; 80, 81, 82; 83, 84, 85; 86, 87, 88; 89, 90, 91; 92, 93, 94; 95, 96, 97; 98, 99, 100; 101, 102, 103; 104, 105, 106; 107, 108, 109; 110, 111, 112; 113, 114, 115; 116, 117, 118; 119, 120, 121; 122, 123, 124; 125, 126, 127; 128, 129, 130; 131, 132, 133; 134, 135, 136; 137, 138, 139; 140, 141, 142; 143, 144, 145; 146, 147, 148; 149, 150, 151; 152, 153, 154; 155, 156, 157; 158, 159, 160; 161, 162, 163; 164, 165, 166; 167, 168, 169; 170, 171, 172; 173, 174, 175; 176, 177, 178; 179, 180, 181; 182, 183, 184; 185, 186, 187; 188, 189, 190; 191, 192, 193; 194, 195, 196; 197, 198, 199; 200, 201, 202; 203, 204, 205; 206, 207, 208; 209, 210, 211; 212, 213, 214; 215, 216, 217; 218, 219, 220; 221, 222, 223; 224, 225, 226; 227, 228, 229; 230, 231, 232; 233, 234, 235; 236, 237, 238; 239, 240, 241; 242, 243, 244; 245, 246, 247; 248, 249, 250; 251, 252, 253; 254, 255, 256; 257, 258, 259; 260, 261, 262; 263, 264, 265; 266, 267, 268; 269, 270, 271; 272, 273, 274; 275, 276, 277; 278, 279, 280; or 281, 282, 283;
a light chain variable domain (VL) CDR1 to CDR3 comprising the amino acid sequence of any one of SEQ ID NOs: 4, 5, 6; 18, 19, 20; 284, 285, 286; 287, 288, 289; 290, 291, 292; 293, 294, 295; 296, 297, 298; 299, 300, 301; 302, 303, 304; 305, 306, 307; 308, 309, 310; 311, 312, 313; 314, 315, 316; 317, 318, 319; 320, 321, 322; 323, 324, 325; 326, 327, 328; 329, 330, 331; 332, 333, 334; 335, 336, 337; 338, 339, 340; 341, 342, 343; 344, 345, 346; 347, 348, 349; 350, 351, 352; 353, 354, 355; 356, 357, 358; 359, 360, 361; 362, 363, 364; 365, 366, 367; 368, 369, 370; 371, 372, 373; 374, 375, 376; 377, 378, 379; 380, 381, 382; 383, 384, 385; 386, 387, 388; 389, 390, 391; 392, 393, 394; 395, 396, 397; 398, 399, 400; 401, 402, 403; 404, 405, 406; 407, 408, 409; 410, 411, 412; 413, 414, 415; 416, 417, 418; 419, 420, 421; 422, 423, 424; 425, 426, 427; 428, 429, 430; 431, 432, 433; 434, 435, 436; 437, 438, 439; 440, 441, 442; 443, 444, 445; 446, 447, 448; 449, 450, 451; 452, 453, 454; 455, 456, 457; 458, 459, 460; 461, 462, 463; 464, 465, 466; 467, 468, 469; 470, 471, 472; 473, 474, 475; 476, 477, 478; 479, 480, 481; 482, 483, 484; 485, 486, 487; 488, 489, 490; 491, 492, 493; 494, 495, 496; 497, 498, 499; 500, 501, 502; 503, 504, 505; 506, 507, 508; 509, 510, 511; 512, 513, 514; 515, 516, 517; 518, 519, 520; 521, 522, 523; 524, 525, 526; 527, 528, 529; 530, 531, 532; 533, 534, 535; or 536, 537, 538.

17. The epitope-targeted, conditionally-activated, pro-drug, antibody of claim 15, wherein the antibody comprises a VH and VL pair comprising the amino acid sequence of any one of the following SEQ ID NOS: 7 and 8, 9 and 10, 21 and 22, or 23 and 24.

18. The epitope-targeted, conditionally-activated, pro-drug, antibody of claim 15, wherein the antibody is a monoclonal antibody.

19. The epitope-targeted, conditionally-activated, pro-drug, antibody of claim 15, wherein the antibody is a full-length antibody.

20. The epitope-targeted, conditionally-activated, pro-drug, antibody of claim 15, wherein the antibody is an antibody fragment.

21. The epitope-targeted, conditionally-activated, pro-drug, antibody of claim 15, wherein the antibody is fused to an Fc domain of any one of the following: human IgG1, human IgG2, human IgG3, and human IgG4.

22. The epitope-targeted, conditionally-activated, pro-drug, antibody of claim 15, wherein the antibody specifically binds to: 4-1BBL, AFP, AKAP-4, ALK, androgen receptor, bcr-abl, Clorf186, CA-125, CA6, CA6, CA19-9, CAMPATH-1, Carbonic anhydrase IX, carcinoembryonic antigen (CEA), CCR8, CD1, CD2, CD3, CD4, CD5, CD8, CD16A, CD19, CD1A, CD20, CD204, CD204, CD204, CD206, CD206, CD206, CD25, CD276, CD28, CD30, CD301, CD301, CD32A, CD32B, CD33, CD36, CD37, CD39, CD40, CD45, CD47, CD5, CD64, CD73, CEA, CLEC4C, CLDN16, CLDN6, CLDN18.2, CLEC10A, CLEC12A, CLEC5A, CLEC9A, CMET, CTCFL, cyclin B1, CYP1B1, DC-SIGN, DEC-205, Dectin1, Dectin2, DLL3, EGFR, EGFRvIII, Endoglin, endosialin, EPCAM, EphA2, epidermal growth factor, ERG, ETV6-AML, ferritin, fibroblast activation protein (FAP), FLT3, folate-binding protein, Fos-related antigen 1, FRA, fucosyl GM1, G250, GD2, GD3, Glycoprotein A33, GloboH, GLP-3, GM2, GM3, gp100, HER2, HER2/neu, HER3, HLD-DR, HMWMAA, HPV E6, HPV E7, hTERT, HVEM, IL-2 receptor, IV1, Latent-TGFB, LCK, Legumain, Ley, LIV1, LMP2, LY6E, MAD-CT-1, MAD-CT-2, MAGE A1, MAGE A3, mannose scavenger receptor1, MARCO, MelanA/MART1, mesothelin (MSLN), metalloproteinase, ML-IAP, MSLN, MUC1, MUC15, MUC16, MYCN, NA17, NAPI2B, NAPI2B, NY-BR-1, NY-ESO-1, OX40L, OY-TES1, p185HER2, P53 mutant, P53 nonmutant, PAGE4, PAP, PAX3, PAX5, PDGFR-B, PDL, PDL1, PLAV1, polysialic acid, PR1, PSA, PSCA, PSMA, PTK7, Ras mutant, RGS5, RhoC, RON, ROR1, ROR2, RRC15, SART3, SIRPA, Sperm protein 17, SSX2, STn, survivin, TAG-72, tenascin, TGFB, Tie 3, TMEM238, TMPRSS3, TMPRSS4, Tn, TRA6, TROP2, TRP-2, tyrosinase, UPK1B, vascular endothelial growth factor, VEGFR, VEGFR2, VISTA, VTCN1, WT1, XAGE 1, or Y6E.

23. The epitope-targeted, conditionally-activated, pro-drug, antibody of claim 15, wherein the antibody is ABT806 (EGFRvIII), adecatumumab (EPCAM), alemtuzumab (CD33), AMG595 (EGFRvIII), anetumab (MSLN), anti-ACE2, anti-EphA2 (EphA2), anti-hyaluronidase, anti-neuraminidase, anti-NY-ESO-1, anti-PTK7 (PTK7), cetuximab (EGFR), cirmtuzumab (ROR1), clivatuzumab (MUC1), CT-011 (PD1), DS-8895a variant 1 (EphA2), DS-8895a variant 2 (EphA2), durvalumab (PDL1) anti-MAGE-A3, edrecolomab (EPCAM), farletuzumab (FRA/folate receptor alpha), Gemtuzumab ozogamicin (CD33), huDS6 (CA6), humanized Ab 2-3 (CEA), humanized PR1A3 (CEA), ibritumomab tiuxetan (CD52), IMAB362/claudiximab (Claudin18.2), ipilimumab (CTLA4), J591 variant 1 (PSMA), J591 variant 2 (PSMA), ladiratuzumab (LIV1), lifastuzumab (NAPI2B), MEDI-547 (EphA2), mirvetuximab (FRA), narnatumab (RON), nimotuzumab (EGFR), nivolumab (PD1), onartuzumab (c-MET), panitumumab (EGFR), patritumab (HER3), pembrolizumab (PD1), pertuzumab (HER2/neu), PF-06647020 (PTK7), RG7841 (LY6E), rituximab (CD20), rovalpituzumab (DLL3), sacituzumab (TROP2), sibrotuzumab (FAP), sofituzumab (MUC16), tositumomab (CD20), trastuzumab (HER2/neu), tremelimumab (CP-675,206)(CTLA4), or zalutumumab (EGFR).

24. The epitope-targeted, conditionally-activated, pro-drug, antibody of claim 15, wherein the linker is a peptide cleaved by actinidain, activated protein C, ADAM10, ADAM12, ADAMS, bromelain, bromelain, calpain, Caspase, caspase-3, Cathepsin, Cathepsin B, Cathepsin C, Cathepsin D, Cathepsin E, cathepsin G, Cathepsin K, Cathepsin L, Chymase, chymosin, chymotrypsin-like protease, CMV protease, collagenase, DESC1, dipeptidyl peptidase, dipeptidyl peptidase IV (DPPIV/CD26), disintegrin and metalloproteinase (ADAM), DPP-4, Elastase, elastase-like protease, enterokinase, Factor Xa, FAP, FAP (FAP-α), Granzyme B, Guanidinobenzoatase, Hepsin, HIV-1 protease, hK1, hK15, hK3, HSV protease, HtrAl, HumNeutrophil Elastase, interleukin-1β converting enzyme, kallikrein, kallikrein-related peptidase (KLK), Lactoferrin, legumain, Marapsin, mast cell chymase, mast cell tryptase, matriptase, Matriptase-2, matrix metalloprotease (MMP), metalloendopeptidase, metalloexopeptidase, Mirl-CP, metalloproteases (MMP1, MMP2, MMP3, MMP7, MMP8, MMP9, MMP10, MMP11, MMP12, MMP13, MMP14), nepenthesin, neutrophil elastase, neutrophil serine protease 4, NS3/4A, PACE4, papain, pepsin, plasmepsin, plasmin, prostate-specific antigen (PSA), proteinase 3, renin, secretase, stromelysin, subtilisin-like protease, thrombin, tissue plasminogen activator (tPA), transmembrane Serine Protease (TMPRSS), trypsin, trypsin-like protease, tryptase, type II transmembrane serine protease (TTSP), Type IV collagenase, urokinase plasminogen activator (uPA), or urokinase plasminogen activator receptor (uPAR).

25. The epitope-targeted, conditionally-activated, pro-drug, antibody of claim 15, wherein the linker comprises non-amino acid cleavage polymers or linkers selected from nucleic acids, lipids, carbohydrates, or chemical linkers, such as those that are cleaved or dissociated by radiation, electromagnetic, pH, chemically, or enzymatically.

26. The epitope-targeted, conditionally-activated, pro-drug, antibody of claim 15, wherein the linker comprises a structure-switch linker that includes amino acid or non-amino acid polymers that adopt an inactivated prodrug conformation with the mask blocking the antibody-antigen binding site and upon exposure to specific microenvironmental conditions the linker switches to an activated antibody conformation.

27. The epitope-targeted, conditionally-activated, pro-drug, antibody of claim 26, wherein the microenvironmental conditions are selected from pH, enzyme(s), or metabolite(s).

28. A method of treating a disease in a subject in need thereof, comprising administering to the subject a therapeutically effective amount of the epitope-targeted, conditionally-activated, pro-drug, antibody comprising:
an antigen binding site with complementarity determining regions (CDRs) from an epitope-specific antibody identified from an in vivo, in vitro, or in silico antibody library; and
one or more engineered epitope masks connected to a linker, wherein the linker comprises a peptide, a polymer, or a chemical-linker that is cleavable in vivo at a target site by an enzyme or cleaved chemically, and wherein the one or more engineered epitope masks binds the epitope-specific antibody at the CDRs of the epitope-specific antibody; and
wherein the epitope-targeted, conditionally-activated, pro-drug, antibody is a fusion protein or an antibody with the one or more engineered epitope masks linked to the antibody via the linker,
wherein the one or more engineered epitope masks are conditionally bound to the CDRs of the epitope-specific antibody, and
wherein cleavage of the linker releases the one or more engineered epitope masks from the antigen binding site from the epitope-targeted, conditionally-activated, pro-drug, antibody.

29. The method of claim 28, where the disease is an autoimmune disease, an inflammatory disease, an infectious disease, or a cancer.

30. The method of claim 29, wherein the cancer is selected from: acute lymphoblastic leukemia, acute myelogenous leukemia, adrenal glands cancer, bladder cancer, bone marrow cancer, breast cancer, chronic lymphocytic leukemia, chronic myeloid leukemia, central nervous system cancer, colorectal cancer, colorectal carcinoma, endometrial cancer, endometrial carcinoma, gastric cancer, gut carcinoma, head and neck squamous cell carcinoma, hematopoietic malignancies, liver cancer, lung carcinoma, lymph node cancer, melanoma, metastatic colorectal cancer, ovarian cancer, pancreatic adenocarcinoma, pituitary tumor, prostate cancer, prostate carcinoma, renal cell carcinoma, retinal cancer, sarcoma, skin cancer, spleen cancer, stomach cancer, thymus cancer, or thyroid cancer.

31. The method of claim 28, wherein the subject is human.

32. A nucleic acid encoding an epitope-targeted, conditionally-activated, pro-drug, antibody comprising:
a nucleic acid encoding an antigen binding site with complementarity determining regions (CDRs) from an antibody identified from an in vivo, in vitro, or in silico antibody library; and
one or more engineered epitope masks linker connected to a linker, wherein the linker comprises a peptide, a polymer, or a chemical-linker that is cleavable in vivo at a target site by an enzyme or cleaved chemically, and wherein the one or more engineered epitope masks binds the epitope-specific antibody at the CDRs of the epitope-targeted antibody; and
wherein the epitope-targeted, conditionally-activated, pro-drug, antibody is a fusion protein or an antibody with the one or more engineered epitope masks linked to the antibody via the linker,
wherein the one or more engineered epitope masks are conditionally bound to the CDRs of the epitope-specific antibody, and
wherein cleavage of the linker releases the one or more engineered epitope masks from the antigen binding site.

33. The nucleic acid of claim 32, the nucleic acid comprises:
a first polynucleotide encoding a heavy chain variable domain having at least 95, 96, 97, 98, 99, or 100% sequence identity to SEQ ID NOS: 11, 13, 25, 27; and
a second polynucleotide encoding light chain variable domain encoding polynucleotide having at least 95, 96, 97, 98, 99, or 100% sequence identity to SEQ ID NOS: 12, 14, 26, 28.

34. The nucleic acid of claim 33, wherein the antibody is a monoclonal, bispecific, multivalent, multi-specific, diabody, chimeric, scFv antibody, or fragments thereof.

35. The nucleic acid of claim 34, wherein an antibody binding domain is fused to an Fc domain of any one of the following: human IgG1, human IgG2, human IgG3, and human IgG4.

36. The nucleic acid of claim 33, wherein the nucleic acid sequence is optimized for expression in a bacterial, fungal, mammalian, insect, or plant cell.

37. The nucleic acid of claim 33, further defined as comprising a vector.

38. The nucleic acid of claim 33, further defined as comprising a vector in a host cell.


SEQUENCE LISTING (538 sequences)

SEQ ID NO: 1 - protein, 8 aa, synthetic construct
  REGION 1..8: note = Synthetic
GYSFTGYT

SEQ ID NO: 2 - protein, 8 aa, synthetic construct
  REGION 1..8: note = Synthetic
INPYKGVS

SEQ ID NO: 3 - protein, 15 aa, synthetic construct
  REGION 1..15: note = Synthetic
ARSGYYGDSDWYFDV

SEQ ID NO: 4 - protein, 6 aa, synthetic construct
  REGION 1..6: note = Synthetic
QDIRNY

SEQ ID NO: 5 - skipped

SEQ ID NO: 6 - protein, 9 aa, synthetic construct
  REGION 1..9: note = Synthetic
QQGNTLPWT

SEQ ID NO: 7 - protein, 173 aa, synthetic construct
  REGION 1..173: note = Synthetic
CPQYPGSEILWQKWGNKKVCYPRGSKPEDANGGGGSSGGPGPAGMKGLPGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 8 - protein, 107 aa, synthetic construct [sequence omitted]

SEQ ID NO: 9 - protein, 122 aa, synthetic construct [sequence omitted]

SEQ ID NO: 10 - protein, 158 aa, synthetic construct
  REGION 1..158: note = Synthetic
CPQYPGSEILWQKWGNKKVCYPRGSKPEDANGGGGSSGGPGPAGMKGLPGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 11 - DNA, 519 bp, synthetic construct
  misc_feature 1..519: note = Synthetic
  source 1..519: mol_type = other DNA
tgcccccagtaccccggctccgagatcctgtggcagaagtggggcaacaagaaggtgtgctaccccaggggctccaagcccgaggacgccaacggcggcggcggctcctccggcggccccggccccgccggcatgaagggcctgcccggctccgaggtgcagctggtggagtccggcggcggcctggtgcagcccggcggctccctgaggctgtcctgcgccgcctccggctactccttcaccggctacaccatgaactgggtgaggcaggcccccggcaagggcctggagtgggtggccctgatcaacccctacaagggcgtgtccacctacaaccagaagttcaaggacaggttcaccatctccgtggacaagtccaagaacaccgcctacctgcagatgaactccctgagggccgaggacaccgccgtgtactactgcgccaggtccggctactacggcgactccgactggtacttcgacgtgtggggccagggcaccctggtgaccgtgtcctcc

SEQ ID NO: 12 - DNA, 321 bp, synthetic construct
  misc_feature 1..321: note = Synthetic
  source 1..321: mol_type = other DNA
gacatccagatgacccagtccccctcctccctgtccgcctccgtgggcgacagggtgaccatcacctgcagggcctcccaggacatcaggaactacctgaactggtaccagcagaagcccggcaaggcccccaagctgctgatctactacacctccaggctggagtccggcgtgccctccaggttctccggctccggctccggcaccgactacaccctgaccatctcctccctgcagcccgaggacttcgccacctactactgccagcagggcaacaccctgccctggaccttcggccagggcaccaaggtggagatcaag

SEQ ID NO: 13 - DNA, 366 bp, synthetic construct
  misc_feature 1..366: note = Synthetic
  source 1..366: mol_type = other DNA
gaggtgcagctggtggagtccggcggcggcctggtgcagcccggcggctccctgaggctgtcctgcgccgcctccggctactccttcaccggctacaccatgaactgggtgaggcaggcccccggcaagggcctggagtgggtggccctgatcaacccctacaagggcgtgtccacctacaaccagaagttcaaggacaggttcaccatctccgtggacaagtccaagaacaccgcctacctgcagatgaactccctgagggccgaggacaccgccgtgtactactgcgccaggtccggctactacggcgactccgactggtacttcgacgtgtggggccagggcaccctggtgaccgtgtcctcc

SEQ ID NO: 14 - DNA, 474 bp, synthetic construct
  misc_feature 1..474: note = Synthetic
  source 1..474: mol_type = other DNA
tgcccccagtaccccggctccgagatcctgtggcagaagtggggcaacaagaaggtgtgctaccccaggggctccaagcccgaggacgccaacggcggcggcggctcctccggcggccccggccccgccggcatgaagggcctgcccggctccgacatccagatgacccagtccccctcctccctgtccgcctccgtgggcgacagggtgaccatcacctgcagggcctcccaggacatcaggaactacctgaactggtaccagcagaagcccggcaaggcccccaagctgctgatctactacacctccaggctggagtccggcgtgccctccaggttctccggctccggctccggcaccgactacaccctgaccatctcctccctgcagcccgaggacttcgccacctactactgccagcagggcaacaccctgccctggaccttcggccagggcaccaaggtggagatcaag

SEQ ID NO: 15 - protein, 9 aa, synthetic construct
  REGION 1..9: note = Synthetic
GYSITSDYA

SEQ ID NO: 16 - protein, 7 aa, synthetic construct
  REGION 1..7: note = Synthetic
ISYSGST

SEQ ID NO: 17 - protein, 7 aa, synthetic construct
  REGION 1..7: note = Synthetic
ATLGLDY

SEQ ID NO: 18 - protein, 12 aa, synthetic construct
  REGION 1..12: note = Synthetic
QSLLYSSNQKNY

SEQ ID NO: 19 - skipped

SEQ ID NO: 20 - protein, 8 aa, synthetic construct
  REGION 1..8: note = Synthetic
QQYHSYRT

SEQ ID NO: 21 - protein, 163 aa, synthetic construct
  REGION 1..163: note = Synthetic
NFTLDRSSVLVDGYSPNRNEPLTGNSDLPGGGGSSGGPGPAGMKGLPGSQVQLQESGPGLVKPSQTLSLTCTVSGYSITSDYAWNWIRQPPGKGLEWIGYISYSGSTIYNPSLKSRISISVDTSKNQFSLKLNSVTAADTAVYYCATLGLDYWGQGTLVTVSS

SEQ ID NO: 22 - protein, 112 aa, synthetic construct
  REGION 1..112: note = Synthetic
DIVMTQSPDSLAVSLGERATINCKSSQSLLYSSNQKNYLAWYQQKPGQPPKLLIYWASTRGSGVPDRFSGSGSGTDFTLTISSLQAEDVAVYYCQQYHSYRTFGQGTKVEIK

SEQ ID NO: 23 - protein, 114 aa, synthetic construct
  REGION 1..114: note = Synthetic
QVQLQESGPGLVKPSQTLSLTCTVSGYSITSDYAWNWIRQPPGKGLEWIGYISYSGSTIYNPSLKSRISISVDTSKNQFSLKLNSVTAADTAVYYCATLGLDYWGQGTLVTVSS

SEQ ID NO: 24 - protein, 156 aa, synthetic construct
  REGION 1..156: note = Synthetic
NFTLDRSSVLVDGYSPNRNEPLTGNSDLPSGGPGPAGMKGLPGSDIVMTQSPDSLAVSLGERATINCKSSQSLLYSSNQKNYLAWYQQKPGQPPKLLIYWASTRGSGVPDRFSGSGSGTDFTLTISSLQAEDVAVYYCQQYHSYRTFGQGTKVEIK

SEQ ID NO: 25 - DNA, 489 bp, synthetic construct
  misc_feature 1..489: note = Synthetic
  source 1..489: mol_type = other DNA
aacttcaccctggacaggtcctccgtgctggtggacggctactcccccaacaggaacgagcccctgaccggcaactccgacctgcccggcggcggcggctcctccggcggccccggccccgccggcatgaagggcctgcccggctcccaggtgcagctgcaggagtccggccccggcctggtgaagccctcccagaccctgtccctgacctgcaccgtgtccggctactccatcacctccgactacgcctggaactggatcaggcagccccccggcaagggcctggagtggatcggctacatctcctactccggctccaccatctacaacccctccctgaagtccaggatctccatctccgtggacacctccaagaaccagttctccctgaagctgaactccgtgaccgccgccgacaccgccgtgtactactgcgccaccctgggcctggactactggggccagggcaccctggtgaccgtgtcctcc

SEQ ID NO: 26 - DNA, 336 bp, synthetic construct
  misc_feature 1..336: note = Synthetic
  source 1..336: mol_type = other DNA
gacatcgtgatgacccagtcccccgactccctggccgtgtccctgggcgagagggccaccatcaactgcaagtcctcccagtccctgctgtactcctccaaccagaagaactacctggcctggtaccagcagaagcccggccagccccccaagctgctgatctactgggcctccaccaggggctccggcgtgcccgacaggttctccggctccggctccggcaccgacttcaccctgaccatctcctccctgcaggccgaggacgtggccgtgtactactgccagcagtaccactcctacaggaccttcggccagggcaccaaggtggagatcaag

SEQ ID NO: 27 - DNA, 342 bp, synthetic construct
  misc_feature 1..342: note = Synthetic
  source 1..342: mol_type = other DNA
caggtgcagctgcaggagtccggccccggcctggtgaagccctcccagaccctgtccctgacctgcaccgtgtccggctactccatcacctccgactacgcctggaactggatcaggcagccccccggcaagggcctggagtggatcggctacatctcctactccggctccaccatctacaacccctccctgaagtccaggatctccatctccgtggacacctccaagaaccagttctccctgaagctgaactccgtgaccgccgccgacaccgccgtgtactactgcgccaccctgggcctggactactggggccagggcaccctggtgaccgtgtcctcc

SEQ ID NO: 28 - DNA, 468 bp, synthetic construct
  misc_feature 1..468: note = Synthetic
  source 1..468: mol_type = other DNA
aacttcaccctggacaggtcctccgtgctggtggacggctactcccccaacaggaacgagcccctgaccggcaactccgacctgccctccggcggccccggccccgccggcatgaagggcctgcccggctccgacatcgtgatgacccagtcccccgactccctggccgtgtccctgggcgagagggccaccatcaactgcaagtcctcccagtccctgctgtactcctccaaccagaagaactacctggcctggtaccagcagaagcccggccagccccccaagctgctgatctactgggcctccaccaggggctccggcgtgcccgacaggttctccggctccggctccggcaccgacttcaccctgaccatctcctccctgcaggccgaggacgtggccgtgtactactgccagcagtaccactcctacaggaccttcggccagggcaccaaggtggagatcaag

SEQ ID NO: 29 - protein, 8 aa, synthetic construct
  REGION 1..8: note = Synthetic
GYTFNIYS

SEQ ID NO: 30 - protein, 10 aa, synthetic construct
  REGION 1..10: note = Synthetic
IRSKYNNYAT

SEQ ID NO: 31 - protein, 16 aa, synthetic construct
  REGION 1..16: note = Synthetic
VRHGNFGNSYVSWFAY

SEQ ID NO: 32 - protein, 8 aa, synthetic construct
  REGION 1..8: note = Synthetic
GFGFNAYA

SEQ ID NO: 33 - protein, 10 aa, synthetic construct
  REGION 1..10: note = Synthetic
IRSKYNNYAT

SEQ ID NO: 34 - protein, 16 aa, synthetic construct
  REGION 1..16: note = Synthetic
VRHGNFGNSYVSWFAY

SEQ ID NO: 35 - protein, 8 aa, synthetic construct
  REGION 1..8: note = Synthetic
RFTFETYA

SEQ ID NO: 36 - protein, 10 aa, synthetic construct
  REGION 1..10: note = Synthetic
IRSKYNNYAT

SEQ ID NO: 37 - protein, 16 aa, synthetic construct
  REGION 1..16: note = Synthetic
VRHGNFGNSYVSWFAY

SEQ ID NO: 38 - protein, 8 aa, synthetic construct
  REGION 1..8: note = Synthetic
GFAFNAYA

SEQ ID NO: 39 - protein, 10 aa, synthetic construct
  REGION 1..10: note = Synthetic
IRSKYNNYAT

SEQ ID NO: 40 - protein, 16 aa, synthetic construct
  REGION 1..16: note = Synthetic
VRHGNFGNSYVSWFAY

SEQ ID NO: 41 - protein, 8 aa, synthetic construct
  REGION 1..8: note = Synthetic
DFTFETYA

SEQ ID NO: 42 - protein, 10 aa, synthetic construct
  REGION 1..10: note = Synthetic
IRSKYNNYAT

SEQ ID NO: 43 - protein, 16 aa, synthetic construct
  REGION 1..16: note = Synthetic
VRHGNFGNSYVSWFAY

SEQ ID NO: 44 - protein, 8 aa, synthetic construct
  REGION 1..8: note = Synthetic
GFAFNVYA

SEQ ID NO: 45 - protein, 10 aa, synthetic construct
  REGION 1..10: note = Synthetic
IRSKYNNYAT

SEQ ID NO: 46 - protein, 16 aa, synthetic construct
  REGION 1..16: note = Synthetic
VRHGNFGNSYVSWFAY

SEQ ID NO: 47 - protein, 8 aa, synthetic construct
  REGION 1..8: note = Synthetic
GFDFNAYA

SEQ ID NO: 48 - protein, 10 aa, synthetic construct
  REGION 1..10: note = Synthetic
IRSKYNNYAT

SEQ ID NO: 49 - protein, 16 aa, synthetic construct
  REGION 1..16: note = Synthetic
VRHGNFGNSYVSWFAY

SEQ ID NO: 50 - protein, 8 aa, synthetic construct
  REGION 1..8: note = Synthetic
GFTFNIQA

SEQ ID NO: 51 - protein, 10 aa, synthetic construct
  REGION 1..10: note = Synthetic
IRSKYNNYAT

SEQ ID NO: 52 - protein, 16 aa, synthetic construct
  REGION 1..16: note = Synthetic
VRHGNFGNSYVSWFAY

SEQ ID NO: 53 - protein, 8 aa, synthetic construct
  REGION 1..8: note = Synthetic
GFTFETYA

SEQ ID NO: 54 - protein, 10 aa, synthetic construct
  REGION 1..10: note = Synthetic
IRSKYNNYAT

SEQ ID NO: 55 - protein, 16 aa, synthetic construct
  REGION 1..16: note = Synthetic
VRHGNFGNSYVSWFAY

SEQ ID NO: 56 - protein, 8 aa, synthetic construct
  REGION 1..8: note = Synthetic
GFSFVTYA

SEQ ID NO: 57 - protein, 10 aa, synthetic construct
  REGION 1..10: note = Synthetic
IRSKYNNYAT

SEQ ID NO: 58 - protein, 16 aa, synthetic construct
  REGION 1..16: note = Synthetic
VRHGNFGNSYVSWFAY

SEQ ID NO: 59 - protein, 8 aa, synthetic construct
  REGION 1..8: note = Synthetic
EFTFETYA

SEQ ID NO: 60 - protein, 10 aa, synthetic construct
  REGION 1..10: note = Synthetic
IRSKYNNYAT

SEQ ID NO: 61 - protein, 16 aa, synthetic construct
  REGION 1..16: note = Synthetic
VRHGNFGNSYVSWFAY

SEQ ID NO: 62 - protein, 8 aa, synthetic construct
  REGION 1..8: note = Synthetic
GFGFNVYA

SEQ ID NO: 63 - protein, 10 aa, synthetic construct
  REGION 1..10: note = Synthetic
IRSKYNNYAT

SEQ ID NO: 64 - protein, 16 aa, synthetic construct
  REGION 1..16: note = Synthetic
VRHGNFGNSYVSWFAY

SEQ ID NO: 65 - protein, 8 aa, synthetic construct
  REGION 1..8: note = Synthetic
GFTFYTYI

SEQ ID NO: 66 - protein, 10 aa, synthetic construct
  REGION 1..10: note = Synthetic
IRSKYNNYAT

SEQ ID NO: 67 - protein, 16 aa, synthetic construct
  REGION 1..16: note = Synthetic
VRHGNFGNSYVSWFAY

SEQ ID NO: 68 - protein, 8 aa, synthetic construct
  REGION 1..8: note = Synthetic
GYTFNVYA

SEQ ID NO: 69 - protein, 10 aa, synthetic construct
  REGION 1..10: note = Synthetic
IRSKYNNYAT

SEQ ID NO: 70 - protein, 16 aa, synthetic construct
  REGION 1..16: note = Synthetic
VRHGNFGNSYVSWFAY

SEQ ID NO: 71 - protein, 8 aa, synthetic construct
  REGION 1..8: note = Synthetic
GFTFNPYA

SEQ ID NO: 72 - protein, 10 aa, synthetic construct
  REGION 1..10: note = Synthetic
IRSKYNNYAT

SEQ ID NO: 73 - protein, 16 aa, synthetic construct
  REGION 1..16: note = Synthetic
VRHGNFGNSYVSWFAY

SEQ ID NO: 74 - protein, 8 aa, synthetic construct
  REGION 1..8: note = Synthetic
GFTFTTYA

SEQ ID NO: 75 - protein, 10 aa, synthetic construct
  REGION 1..10: note = Synthetic
IRSKYNNYAT

SEQ ID NO: 76 - protein, 16 aa, synthetic construct
  REGION 1..16: note = Synthetic
VRHGNFGNSYVSWFAY

SEQ ID NO: 77 - protein, 8 aa, synthetic construct
  REGION 1..8: note = Synthetic
GFTFNTYA

SEQ ID NO: 78 - protein, 10 aa, synthetic construct
  REGION 1..10: note = Synthetic
IRSKRSNYAT

SEQ ID NO: 79 - protein, 16 aa, synthetic construct
  REGION 1..16: note = Synthetic
VRHGNFGNSYVSWFAY

SEQ ID NO: 80 - protein, 8 aa, synthetic construct
  REGION 1..8: note = Synthetic
GFTFNTYA

SEQ ID NO: 81 - protein, 10 aa, synthetic construct
  REGION 1..10: note = Synthetic
IRSKGNNYAT

SEQ ID NO: 82 - protein, 16 aa, synthetic construct
  REGION 1..16: note = Synthetic
VRHGNFGNSYVSWFAY

SEQ ID NO: 83 - protein, 8 aa, synthetic construct
  REGION 1..8: note = Synthetic
GFTFNTYA

SEQ ID NO: 84 - protein, 10 aa, synthetic construct
  REGION 1..10: note = Synthetic
IRTKSNNYAV

SEQ ID NO: 85 - protein, 16 aa, synthetic construct
  REGION 1..16: note = Synthetic
VRHGNFGNSYVSWFAY

SEQ ID NO: 86 - protein, 8 aa, synthetic construct
  REGION 1..8: note = Synthetic
GFTFNTYA

SEQ ID NO: 87 - protein, 10 aa, synthetic construct
  REGION 1..10: note = Synthetic
IKTKHDNYAT

SEQ ID NO: 88 - protein, 16 aa, synthetic construct
  REGION 1..16: note = Synthetic
VRHGNFGNSYVSWFAY

SEQ ID NO: 89 - protein, 8 aa, synthetic construct
  REGION 1..8: note = Synthetic
GFTFNTYA

SEQ ID NO: 90 - protein, 10 aa, synthetic construct
  REGION 1..10: note = Synthetic
IRSKNFNYAT

SEQ ID NO: 91 - protein, 16 aa, synthetic construct
  REGION 1..16: note = Synthetic
VRHGNFGNSYVSWFAY

SEQ ID NO: 92 - protein, 8 aa, synthetic construct
  REGION 1..8: note = Synthetic
GFTFNTYA

SEQ ID NO: 93 - protein, 10 aa, synthetic construct
  REGION 1..10: note = Synthetic
VRSKQTNYAT

SEQ ID NO: 94 - protein, 16 aa, synthetic construct
  REGION 1..16: note = Synthetic
VRHGNFGNSYVSWFAY

SEQ ID NO: 95 - protein, 8 aa, synthetic construct
  REGION 1..8: note = Synthetic
GFTFNTYA

SEQ ID NO: 96 - protein, 10 aa, synthetic construct
  REGION 1..10: note = Synthetic
IRSKHNNYET

SEQ ID NO: 97 - protein, 16 aa, synthetic construct
  REGION 1..16: note = Synthetic
VRHGNFGNSYVSWFAY

SEQ ID NO: 98 - protein, 8 aa, synthetic construct
  REGION 1..8: note = Synthetic
GFTFNTYA

SEQ ID NO: 99 - protein, 10 aa, synthetic construct
  REGION 1..10: note = Synthetic
IRSYINNYAT

SEQ ID NO: 100 - protein, 16 aa, synthetic construct
  REGION 1..16: note = Synthetic
VRHGNFGNSYVSWFAY

SEQ ID NO: 101 - protein, 8 aa, synthetic construct
  REGION 1..8: note = Synthetic
GFTFNTYA

SEQ ID NO: 102 - protein, 10 aa, synthetic construct
  REGION 1..10: note = Synthetic
IRSKVRNYAT

SEQ ID NO: 103 - protein, 16 aa, synthetic construct
  REGION 1..16: note = Synthetic
VRHGNFGNSYVSWFAY

SEQ ID NO: 104 - protein, 8 aa, synthetic construct
  REGION 1..8: note = Synthetic
GFTFNTYA

SEQ ID NO: 105 - protein, 10 aa, synthetic construct
  REGION 1..10: note = Synthetic
VRSKVNNYAT

SEQ ID NO: 106 - protein, 16 aa, synthetic construct
  REGION 1..16: note = Synthetic
VRHGNFGNSYVSWFAY

SEQ ID NO: 107 - protein, 8 aa, synthetic construct
  REGION 1..8: note = Synthetic
GFTFNTYA

SEQ ID NO: 108 - protein, 10 aa, synthetic construct
  REGION 1..10: note = Synthetic
IRSKSGNYAT

SEQ ID NO: 109 - protein, 16 aa, synthetic construct
  REGION 1..16: note = Synthetic
VRHGNFGNSYVSWFAY

SEQ ID NO: 110 - protein, 8 aa, synthetic construct
  REGION 1..8: note = Synthetic
GFTFNTYA

SEQ ID NO: 111 - protein, 10 aa, synthetic construct
  REGION 1..10: note = Synthetic
IRSKPNEYAT

SEQ ID NO: 112 - protein, 16 aa, synthetic construct
  REGION 1..16: note = Synthetic
VRHGNFGNSYVSWFAY

SEQ ID NO: 113 - protein, 8 aa, synthetic construct
  REGION 1..8: note = Synthetic
GFTFNTYA

SEQ ID NO: 114 - protein, 10 aa, synthetic construct
  REGION 1..10: note = Synthetic
IRSKFKNYAT

SEQ ID NO: 115 - protein, 16 aa, synthetic construct
  REGION 1..16: note = Synthetic
VRHGNFGNSYVSWFAY

SEQ ID NO: 116 - protein, 8 aa, synthetic construct
  REGION 1..8: note = Synthetic
GFTFNTYA

SEQ ID NO: 117 - protein, 10 aa, synthetic construct
  REGION 1..10: note = Synthetic
IRSKYTNYAT

SEQ ID NO: 118 - protein, 16 aa, synthetic construct
  REGION 1..16: note = Synthetic
VRHGNFGNSYVSWFAY

SEQ ID NO: 119 - protein, 8 aa, synthetic construct
  REGION 1..8: note = Synthetic
GFTFNTYA

SEQ ID NO: 120 - protein, 10 aa, synthetic construct
  REGION 1..10: note = Synthetic
IRSQYNNYAT

SEQ ID NO: 121 - protein, 16 aa, synthetic construct
  REGION 1..16: note = Synthetic
VRHGNFGNSYVSWFAY

SEQ ID NO: 122 - protein, 8 aa, synthetic construct
  REGION 1..8: note = Synthetic
GFTFNTYA

SEQ ID NO: 123 - protein, 10 aa, synthetic construct
  REGION 1..10: note = Synthetic
IRSKYHNYAT

SEQ ID NO: 124 - protein, 16 aa, synthetic construct
  REGION 1..16: note = Synthetic
VRHGNFGNSYVSWFAY

SEQ ID NO: 125 - protein, 8 aa, synthetic construct
  REGION 1..8: note = Synthetic
GFTFNTYA

SEQ ID NO: 126 - protein, 10 aa, synthetic construct
  REGION 1..10: note = Synthetic
IRSKVNNYAT

SEQ ID NO: 127 - protein, 16 aa, synthetic construct
  REGION 1..16: note = Synthetic
VRHGNFGNSYVSWFAY

SEQ ID NO: 128 - protein, 8 aa, synthetic construct
  REGION 1..8: note = Synthetic
GFTFNTYA

SEQ ID NO: 129 - protein, 10 aa, synthetic construct
  REGION 1..10: note = Synthetic
IRSSNNNYAT

SEQ ID NO: 130 - protein, 16 aa, synthetic construct
  REGION 1..16: note = Synthetic
VRHGNFGNSYVSWFAY

SEQ ID NO: 131 - protein, 8 aa, synthetic construct
  REGION 1..8: note = Synthetic
GFTFNTYA

SEQ ID NO: 132 - protein, 10 aa, synthetic construct
  REGION 1..10: note = Synthetic
IRSKYNKYAT

SEQ ID NO: 133 - protein, 16 aa, synthetic construct
  REGION 1..16: note = Synthetic
VRHGNFGNSYVSWFAY

SEQ ID NO: 134 - protein, 8 aa, synthetic construct
  REGION 1..8: note = Synthetic
GFTFNTYA

SEQ ID NO: 135 - protein, 10 aa, synthetic construct
  REGION 1..10: note = Synthetic
VRSKASNYAT

SEQ ID NO: 136 - protein, 16 aa, synthetic construct
  REGION 1..16: note = Synthetic
VRHGNFGNSYVSWFAY

SEQ ID NO: 137 - protein, 8 aa, synthetic construct
  REGION 1..8: note = Synthetic
GFTFNTYA

SEQ ID NO: 138 - protein, 10 aa, synthetic construct
  REGION 1..10: note = Synthetic
IRSKSRNYAT

SEQ ID NO: 139 - protein, 16 aa, synthetic construct
  REGION 1..16: note = Synthetic
VRHGNFGNSYVSWFAY

SEQ ID NO: 140 - protein, 8 aa, synthetic construct
  REGION 1..8: note = Synthetic
GFTFNTYA

SEQ ID NO: 141 - protein, 10 aa, synthetic construct
  REGION 1..10: note = Synthetic
IRSKYNNYAT

SEQ ID NO: 142 - protein, 16 aa, synthetic construct
  REGION 1..16: note = Synthetic
VREGGSGNYYNVYFAY

SEQ ID NO: 143 - protein, 8 aa, synthetic construct
  REGION 1..8: note = Synthetic
GFTFNTYA

SEQ ID NO: 144 - protein, 10 aa, synthetic construct
  REGION 1..10: note = Synthetic
IRSKYNNYAT

SEQ ID NO: 145 - protein, 16 aa, synthetic construct
  REGION 1..16: note = Synthetic
VRSGRFGESHVGPFAY

SEQ ID NO: 146 - protein, 8 aa, synthetic construct
  REGION 1..8: note = Synthetic
GFTFNTYA

SEQ ID NO: 147 - protein, 10 aa, synthetic construct
  REGION 1..10: note = Synthetic
IRSKYNNYAT

SEQ ID NO: 148 - protein, 16 aa, synthetic construct
  REGION 1..16: note = Synthetic
ARHGEYSSGWPYYFAY

SEQ ID NO: 149 - protein, 8 aa, synthetic construct
  REGION 1..8: note = Synthetic
GFTFNTYA

SEQ ID NO: 150 - protein, 10 aa, synthetic construct
  REGION 1..10: note = Synthetic
IRSKYNNYAT

SEQ ID NO: 151 - protein, 16 aa, synthetic construct
  REGION 1..16: note = Synthetic
VRHGNFGNVGTSYFAY

SEQ ID NO: 152 - protein, 8 aa, synthetic construct
  REGION 1..8: note = Synthetic
GFTFNTYA

SEQ ID NO: 153 - protein, 10 aa, synthetic construct
  REGION 1..10: note = Synthetic
IRSKYNNYAT

SEQ ID NO: 154 - protein, 16 aa, synthetic construct
  REGION 1..16: note = Synthetic
VREGIGGTSYRGNFAY

SEQ ID NO: 155 - protein, 8 aa, synthetic construct
  REGION 1..8: note = Synthetic
GFTFNTYA

SEQ ID NO: 156 - protein, 10 aa, synthetic construct
  REGION 1..10: note = Synthetic
IRSKYNNYAT

SEQ ID NO: 157 - protein, 16 aa, synthetic construct
  REGION 1..16: note = Synthetic
VRHGNFGNSYVSWFAY

SEQ ID NO: 158 - protein, 8 aa, synthetic construct
  REGION 1..8: note = Synthetic
GFTFNTYA

SEQ ID NO: 159 - protein, 10 aa, synthetic construct
  REGION 1..10: note = Synthetic
IRSKYNNYAT

SEQ ID NO: 160 - protein, 16 aa, synthetic construct
  REGION 1..16: note = Synthetic
VRHGNFGNSYVSWFAY

SEQ ID NO: 161 - protein, 8 aa, synthetic construct
  REGION 1..8: note = Synthetic
GFTFNTYA

SEQ ID NO: 162 - protein, 10 aa, synthetic construct
  REGION 1..10: note = Synthetic
IRSKYNNYAT

SEQ ID NO: 163 - protein, 16 aa, synthetic construct
  REGION 1..16: note = Synthetic
VRHGNFGNSYVSWFAY

SEQ ID NO: 164 - protein, 8 aa, synthetic construct
  REGION 1..8: note = Synthetic
GFTFNTYA

SEQ ID NO: 165 - protein, 10 aa, synthetic construct
  REGION 1..10: note = Synthetic
IRSKYNNYAT

SEQ ID NO: 166 - protein, 16 aa, synthetic construct
  REGION 1..16: note = Synthetic
VRHGNFGNSYVSWFAY

SEQ ID NO: 167 - protein, 8 aa, synthetic construct
  REGION 1..8: note = Synthetic
GFTFNTYA

SEQ ID NO: 168 - protein, 10 aa, synthetic construct
  REGION 1..10: note = Synthetic
IRSKYNNYAT

SEQ ID NO: 169 - protein, 16 aa, synthetic construct
  REGION 1..16: note = Synthetic
VRHGNFGNSYVSWFAY

SEQ ID NO: 170 - protein, 8 aa, synthetic construct
  REGION 1..8: note = Synthetic
GFTFNTYA

SEQ ID NO: 171 - protein, 10 aa, synthetic construct
  REGION 1..10: note = Synthetic
IRSKYNNYAT

SEQ ID NO: 172 - protein, 16 aa, synthetic construct
  REGION 1..16: note = Synthetic
VRHGNFGNSYVSWFAY

SEQ ID NO: 173 - protein, 8 aa, synthetic construct
  REGION 1..8: note = Synthetic
GFTFNTYA

SEQ ID NO: 174 - protein, 10 aa, synthetic construct
  REGION 1..10: note = Synthetic
IRSKYNNYAT

SEQ ID NO: 175 - protein, 16 aa, synthetic construct
  REGION 1..16: note = Synthetic
VRHGNFGNSYVSWFAY

SEQ ID NO: 176 - protein, 8 aa, synthetic construct
  REGION 1..8: note = Synthetic
GFTFNTYA

SEQ ID NO: 177 - protein, 10 aa, synthetic construct
  REGION 1..10: note = Synthetic
IRSKYNNYAT

SEQ ID NO: 178 - protein, 16 aa, synthetic construct
  REGION 1..16: note = Synthetic
VRHGNFGNSYVSWFAY

SEQ ID NO: 179 - protein, 8 aa, synthetic construct
  REGION 1..8: note = Synthetic
GFTFNTYA

SEQ ID NO: 180 - protein, 10 aa, synthetic construct
  REGION 1..10: note = Synthetic
IRSKYNNYAT

SEQ ID NO: 181 - protein, 16 aa, synthetic construct
  REGION 1..16: note = Synthetic
VRHGNFGNSYVSWFAY

SEQ ID NO: 182 - protein, 8 aa, synthetic construct
  REGION 1..8: note = Synthetic
GFTFNTYA

SEQ ID NO: 183 - protein, 10 aa, synthetic construct
  REGION 1..10: note = Synthetic
IRSKYNNYAT

SEQ ID NO: 184 - protein, 16 aa, synthetic construct
  REGION 1..16: note = Synthetic
VRHGNFGNSYVSWFAY

SEQ ID NO: 185 - protein, 8 aa, synthetic construct
  REGION 1..8: note = Synthetic
GFTFNTYA

SEQ ID NO: 186 - protein, 10 aa, synthetic construct
  REGION 1..10: note = Synthetic
IRSKYNNYAT

SEQ ID NO: 187 - protein, 16 aa, synthetic construct
  REGION 1..16: note = Synthetic
VRHGNFGNSYVSWFAY

SEQ ID NO: 188 - protein, 8 aa, synthetic construct
  REGION 1..8: note = Synthetic
GFTFNTYA

SEQ ID NO: 189 - protein, 10 aa, synthetic construct
  REGION 1..10: note = Synthetic
IRSKYNNYAT

SEQ ID NO: 190 - protein, 16 aa, synthetic construct
  REGION 1..16: note = Synthetic
VRHGNFGNSYVSWFAY

SEQ ID NO: 191 - protein, 8 aa, synthetic construct
  REGION 1..8: note = Synthetic
GFTFNTYA

SEQ ID NO: 192 - protein, 10 aa, synthetic construct
  REGION 1..10: note = Synthetic
IRSKYNNYAT

SEQ ID NO: 193 - protein, 16 aa, synthetic construct
  REGION 1..16: note = Synthetic
VRHGNFGNSYVSWFAY

SEQ ID NO: 194 - protein, 8 aa, synthetic construct
  REGION 1..8: note = Synthetic
GFTFNTYA

SEQ ID NO: 195 - protein, 10 aa, synthetic construct
  REGION 1..10: note = Synthetic
IRSKYNNYAT

SEQ ID NO: 196 - protein, 16 aa, synthetic construct
  REGION 1..16: note = Synthetic
VRHGNFGNSYVSWFAY

SEQ ID NO: 197 - protein, 8 aa, synthetic construct
  REGION 1..8: note = Synthetic
GFTFNTYA

SEQ ID NO: 198 - protein, 10 aa, synthetic construct
  REGION 1..10: note = Synthetic
IRSKYNNYAT

SEQ ID NO: 199 - protein, 16 aa, synthetic construct
  REGION 1..16: note = Synthetic
VRHGNFGNSYVSWFAY

SEQ ID NO: 200 - protein, 8 aa, synthetic construct
  REGION 1..8: note = Synthetic
GFTFNTYA

SEQ ID NO: 201 - protein, 10 aa, synthetic construct
  REGION 1..10: note = Synthetic
IRSKYNNYAT

SEQ ID NO: 202 - protein, 16 aa, synthetic construct
  REGION 1..16: note = Synthetic
VRHGNFGNSYVSWFAY

SEQ ID NO: 203 - protein, 8 aa, synthetic construct
  REGION 1..8: note = Synthetic
GFTFNTYA

SEQ ID NO: 204 - protein, 10 aa, synthetic construct
  REGION 1..10: note = Synthetic
IRSKYNNYAT

SEQ ID NO: 205 - protein, 16 aa, synthetic construct
  REGION 1..16: note = Synthetic
VRHGNFGNSYVSWFAY

SEQ ID NO: 206 - protein, 8 aa, synthetic construct
  REGION 1..8: note = Synthetic
GFTFNTYA

SEQ ID NO: 207 - protein, 10 aa, synthetic construct
  REGION 1..10: note = Synthetic
IRSKYNNYAT

SEQ ID NO: 208 - protein, 16 aa, synthetic construct
  REGION 1..16: note = Synthetic
VRHGNFGNSYVSWFAY

SEQ ID NO: 209 - protein, 8 aa, synthetic construct
  REGION 1..8: note = Synthetic
GFTFNTYA

SEQ ID NO: 210 - protein, 10 aa, synthetic construct
  REGION 1..10: note = Synthetic
IRSKYNNYAT

SEQ ID NO: 211 - protein, 16 aa, synthetic construct
  REGION 1..16: note = Synthetic
VRHGNFGNSYVSWFAY

SEQ ID NO: 212 - protein, 8 aa, synthetic construct
  REGION 1..8: note = Synthetic
GFTFNTYA

SEQ ID NO: 213 - protein, 10 aa, synthetic construct
  REGION 1..10: note = Synthetic
IRSKYNNYAT

SEQ ID NO: 214 - protein, 16 aa, synthetic construct
  REGION 1..16: note = Synthetic
VRHGNFGNSYVSWFAY

SEQ ID NO: 215 - protein, 8 aa, synthetic construct
  REGION 1..8: note = Synthetic
GFTFNTYA

SEQ ID NO: 216 - protein, 10 aa, synthetic construct
  REGION 1..10: note = Synthetic
IRSKYNNYAT

SEQ ID NO: 217 - protein, 16 aa, synthetic construct
  REGION 1..16: note = Synthetic
VRHGNFGNSYVSWFAY

SEQ ID NO: 218 - protein, 8 aa, synthetic construct
  REGION 1..8: note = Synthetic
GFTFNTYA

SEQ ID NO: 219 - protein, 10 aa, synthetic construct
  REGION 1..10: note = Synthetic
IRSKYNNYAT

SEQ ID NO: 220 - protein, 16 aa, synthetic construct
  REGION 1..16: note = Synthetic
VRHGNFGNSYVSWFAY

SEQ ID NO: 221 - protein, 8 aa, synthetic construct
  REGION 1..8: note = Synthetic
GFTFNTYA

SEQ ID NO: 222 - protein, 10 aa, synthetic construct
  REGION 1..10: note = Synthetic
IRSKYNNYAT

SEQ ID NO: 223 - protein, 16 aa, synthetic construct
  REGION 1..16: note = Synthetic
VRHGNFGNSYVSWFAY

SEQ ID NO: 224 - protein, 8 aa, synthetic construct
  REGION 1..8: note = Synthetic
GFTFNTYA

SEQ ID NO: 225 - protein, 10 aa, synthetic construct
  REGION 1..10: note = Synthetic
IRSKYNNYAT

SEQ ID NO: 226 - protein, 16 aa, synthetic construct
  REGION 1..16: note = Synthetic
VRHGNFGNSYVSWFAY

SEQ ID NO: 227 - protein, 8 aa, synthetic construct
  REGION 1..8: note = Synthetic
GFTFNTYA

SEQ ID NO: 228 - protein, 10 aa, synthetic construct
  REGION 1..10: note = Synthetic
IRSKYNNYAT

SEQ ID NO: 229 - protein, 16 aa, synthetic construct
  REGION 1..16: note = Synthetic
VRHGNFGNSYVSWFAY

SEQ ID NO: 230 - protein, 8 aa, synthetic construct
  REGION 1..8: note = Synthetic
GFTFNTYA

SEQ ID NO: 231 - protein, 10 aa, synthetic construct
  REGION 1..10: note = Synthetic
IRSKYNNYAT

SEQ ID NO: 232 - protein, 16 aa, synthetic construct
  REGION 1..16: note = Synthetic
VRHGNFGNSYVSWFAY

SEQ ID NO: 233 - protein, 8 aa, synthetic construct
  REGION 1..8: note = Synthetic
GFTFNTYA

SEQ ID NO: 234 - protein, 10 aa, synthetic construct
  REGION 1..10: note = Synthetic
IRSKYNNYAT

SEQ ID NO: 235 - protein, 16 aa, synthetic construct
  REGION 1..16: note = Synthetic
VRHGNFGNSYVSWFAY

SEQ ID NO: 236 - protein, 8 aa, synthetic construct
  REGION 1..8: note = Synthetic
GFTFNTYA

SEQ ID NO: 237 - protein, 10 aa, synthetic construct
  REGION 1..10: note = Synthetic
IRSKYNNYAT

SEQ ID NO: 238 - protein, 16 aa, synthetic construct
  REGION 1..16: note = Synthetic
VRHGNFGNSYVSWFAY

SEQ ID NO: 239 - protein, 8 aa, synthetic construct
  REGION 1..8: note = Synthetic
GFTFNTYA

SEQ ID NO: 240 - protein, 10 aa, synthetic construct
  REGION 1..10: note = Synthetic
IRSKYNNYAT

SEQ ID NO: 241 - protein, 16 aa, synthetic construct
  REGION 1..16: note = Synthetic
VRHGNFGNSYVSWFAY

SEQ ID NO: 242 - protein, 8 aa, synthetic construct
  REGION 1..8: note = Synthetic
GFTFNTYA

SEQ ID NO: 243 - protein, 10 aa, synthetic construct
  REGION 1..10: note = Synthetic
IRSKYNNYAT

SEQ ID NO: 244 - protein, 16 aa, synthetic construct
  REGION 1..16: note = Synthetic
VRHGNFGNSYVSWFAY

SEQ ID NO: 245 - protein, 8 aa, synthetic construct
  REGION 1..8: note = Synthetic
GFTFNTYA

SEQ ID NO: 246 - protein, 10 aa, synthetic construct
  REGION 1..10: note = Synthetic
IRSKYNNYAT

SEQ ID NO: 247 - protein, 16 aa, synthetic construct
  REGION 1..16: note = Synthetic
VRHGNFGNSYVSWFAY

SEQ ID NO: 248 - protein, 8 aa, synthetic construct
  REGION 1..8: note = Synthetic
GFTFNTYA

SEQ ID NO: 249 - protein, 10 aa, synthetic construct
  REGION 1..10: note = Synthetic
IRSKYNNYAT

SEQ ID NO: 250 - protein, 16 aa, synthetic construct
  REGION 1..16: note = Synthetic
VRHGNFGNSYVSWFAY

SEQ ID NO: 251 - protein, 8 aa, synthetic construct
  REGION 1..8: note = Synthetic
GFTFNTYA

SEQ ID NO: 252 - protein, 10 aa, synthetic construct
  REGION 1..10: note = Synthetic
IRSKYNNYAT

SEQ ID NO: 253 - protein, 16 aa, synthetic construct
  REGION 1..16: note = Synthetic
VRHGNFGNSYVSWFAY

SEQ ID NO: 254 - protein, 8 aa, synthetic construct
  REGION 1..8: note = Synthetic
GFTFNTYA

SEQ ID NO: 255 - protein, 10 aa, synthetic construct
  REGION 1..10: note = Synthetic
IRSKYNNYAT

SEQ ID NO: 256 - protein, 16 aa, synthetic construct
  REGION 1..16: note = Synthetic
VRHGNFGNSYVSWFAY

SEQ ID NO: 257 - protein, 8 aa, synthetic construct
  REGION 1..8: note = Synthetic
GFTFNTYA

SEQ ID NO: 258 - protein, 10 aa, synthetic construct
  REGION 1..10: note = Synthetic
IRSKYNNYAT

SEQ ID NO: 259 - protein, 16 aa, synthetic construct
  REGION 1..16: note = Synthetic
VRHGNFGNSYVSWFAY

SEQ ID NO: 260 - protein, 8 aa, synthetic construct
  REGION 1..8: note = Synthetic
GFTFNTYA

SEQ ID NO: 261 - protein, 10 aa, synthetic construct
  REGION 1..10: note = Synthetic
IRSKYNNYAT

SEQ ID NO: 262 - protein, 16 aa, synthetic construct
  REGION 1..16: note = Synthetic
VRHGNFGNSYVSWFAY

SEQ ID NO: 263 - protein, 8 aa, synthetic construct
  REGION 1..8: note = Synthetic
GFTFNTYA

SEQ ID NO: 264 - protein, 10 aa, synthetic construct
  REGION 1..10: note = Synthetic
IRSKYNNYAT

SEQ ID NO: 265 - protein, 16 aa, synthetic construct
  REGION 1..16: note = Synthetic
VRHGNFGNSYVSWFAY

SEQ ID NO: 266 - protein, 8 aa, synthetic construct
  REGION 1..8: note = Synthetic
GFTFNTYA

SEQ ID NO: 267 - protein, 10 aa, synthetic construct
  REGION 1..10: note = Synthetic
IRSKYNNYAT

SEQ ID NO: 268 - protein, 16 aa, synthetic construct
  REGION 1..16: note = Synthetic
VRHGNFGNSYVSWFAY

SEQ ID NO: 269 - protein, 8 aa, synthetic construct
  REGION 1..8: note = Synthetic
GFTFNTYA

SEQ ID NO: 270 - protein, 10 aa, synthetic construct
  REGION 1..10: note = Synthetic
IRSKYNNYAT

SEQ ID NO: 271 - protein, 16 aa, synthetic construct
  REGION 1..16: note = Synthetic
VRHGNFGNSYVSWFAY

SEQ ID NO: 272 - protein, 8 aa, synthetic construct
  REGION 1..8: note = Synthetic
GFTFNTYA

SEQ ID NO: 273 - protein, 10 aa, synthetic construct
  REGION 1..10: note = Synthetic
IRSKYNNYAT

SEQ ID NO: 274 - protein, 16 aa, synthetic construct
  REGION 1..16: note = Synthetic
VRHGNFGNSYVSWFAY

SEQ ID NO: 275 - protein, 8 aa, synthetic construct
  REGION 1..8: note = Synthetic
GFTFNTYA

SEQ ID NO: 276 - protein, 10 aa, synthetic construct
  REGION 1..10: note = Synthetic
IRSKYNNYAT

SEQ ID NO: 277 - protein, 16 aa, synthetic construct
  REGION 1..16: note = Synthetic
VRHGNFGNSYVSWFAY

SEQ ID NO: 278 - protein, 8 aa, synthetic construct
  REGION 1..8: note = Synthetic
GFTFNTYA

SEQ ID NO: 279 - protein, 10 aa, synthetic construct
  REGION 1..10: note = Synthetic
IRSKYNNYAT

SEQ ID NO: 280 - protein, 16 aa, synthetic construct
  REGION 1..16: note = Synthetic
VRHGNFGNSYVSWFAY

SEQ ID NO: 281 - protein, 8 aa, synthetic construct
  REGION 1..8: note = Synthetic
GFDFNAYA

SEQ ID NO: 282 - protein, 10 aa, synthetic construct
  REGION 1..10: note = Synthetic
IRSKYNNYAT

SEQ ID NO: 283 - protein, 16 aa, synthetic construct
  REGION 1..16: note = Synthetic
VRHGNFGNSYVSWFAY

SEQ ID NO: 284 - protein, 9 aa, synthetic construct
  REGION 1..9: note = Synthetic
TGAVTTSNY

SEQ ID NO: 285 - skipped

SEQ ID NO: 286 - protein, 9 aa, synthetic construct
  REGION 1..9: note = Synthetic
ALWYSNLWV

SEQ ID NO: 287 - protein, 9 aa, synthetic construct
  REGION 1..9: note = Synthetic
TGAVTTSNY

SEQ ID NO: 288 - skipped

SEQ ID NO: 289 - protein, 9 aa, synthetic construct
  REGION 1..9: note = Synthetic
ALWYSNLWV

SEQ ID NO: 290 - protein, 9 aa, synthetic construct
  REGION 1..9: note = Synthetic
TGAVTTSNY

SEQ ID NO: 291 - skipped

SEQ ID NO: 292 - protein, 9 aa, synthetic construct
  REGION 1..9: note = Synthetic
ALWYSNLWV

SEQ ID NO: 293 - protein, 9 aa, synthetic construct
  REGION 1..9: note = Synthetic
TGAVTTSNY

SEQ ID NO: 294 - skipped

SEQ ID NO: 295 - protein, 9 aa, synthetic construct
  REGION 1..9: note = Synthetic
ALWYSNLWV

SEQ ID NO: 296 - protein, 9 aa, synthetic construct
  REGION 1..9: note = Synthetic
TGAVTTSNY

SEQ ID NO: 297 - skipped

SEQ ID NO: 298 - protein, 9 aa, synthetic construct
  REGION 1..9: note = Synthetic
ALWYSNLWV

SEQ ID NO: 299 - protein, 9 aa, synthetic construct
  REGION 1..9: note = Synthetic
TGAVTTSNY

SEQ ID NO: 300 - skipped

SEQ ID NO: 301 - protein, 9 aa, synthetic construct
  REGION 1..9: note = Synthetic
ALWYSNLWV

SEQ ID NO: 302 - protein, 9 aa, synthetic construct
  REGION 1..9: note = Synthetic
TGAVTTSNY

SEQ ID NO: 303 - skipped

SEQ ID NO: 304 - protein, 9 aa, synthetic construct
  REGION 1..9: note = Synthetic
ALWYSNLWV

SEQ ID NO: 305 - protein, 9 aa, synthetic construct
  REGION 1..9: note = Synthetic
TGAVTTSNY

SEQ ID NO: 306 - skipped

SEQ ID NO: 307 - protein, 9 aa, synthetic construct
  REGION 1..9: note = Synthetic
ALWYSNLWV

SEQ ID NO: 308 - protein, 9 aa, synthetic construct
  REGION 1..9: note = Synthetic
TGAVTTSNY

SEQ ID NO: 309 - skipped

SEQ ID NO: 310 - protein, 9 aa, synthetic construct
  REGION 1..9: note = Synthetic
ALWYSNLWV

SEQ ID NO: 311 - protein, 9 aa, synthetic construct
  REGION 1..9: note = Synthetic
TGAVTTSNY

SEQ ID NO: 312 - skipped

SEQ ID NO: 313 - protein, 9 aa, synthetic construct
  REGION 1..9: note = Synthetic
ALWYSNLWV

SEQ ID NO: 314 - protein, 9 aa, synthetic construct
  REGION 1..9: note = Synthetic
TGAVTTSNY

SEQ ID NO: 315 - skipped

SEQ ID NO: 316 - protein, 9 aa, synthetic construct
  REGION 1..9: note = Synthetic
ALWYSNLWV

SEQ ID NO: 317 - protein, 9 aa, synthetic construct
  REGION 1..9: note = Synthetic
TGAVTTSNY

SEQ ID NO: 318 - skipped

SEQ ID NO: 319 - protein, 9 aa, synthetic construct
  REGION 1..9: note = Synthetic
ALWYSNLWV

SEQ ID NO: 320 - protein, 9 aa, synthetic construct
  REGION 1..9: note = Synthetic
TGAVTTSNY

SEQ ID NO: 321 - skipped

SEQ ID NO: 322 - protein, 9 aa, synthetic construct
  REGION 1..9: note = Synthetic
ALWYSNLWV

SEQ ID NO: 323 - protein, 9 aa, synthetic construct
  REGION 1..9: note = Synthetic
TGAVTTSNY

SEQ ID NO: 324 - skipped

SEQ ID NO: 325 - protein, 9 aa, synthetic construct
  REGION 1..9: note = Synthetic
ALWYSNLWV

SEQ ID NO: 326 - protein, 9 aa, synthetic construct
  REGION 1..9: note = Synthetic
TGAVTTSNY

SEQ ID NO: 327 - skipped

SEQ ID NO: 328 - protein, 9 aa, synthetic construct
  REGION 1..9: note = Synthetic
ALWYSNLWV

SEQ ID NO: 329 - protein, 9 aa, synthetic construct
  REGION 1..9: note = Synthetic
TGAVTTSNY

SEQ ID NO: 330 - skipped

SEQ ID NO: 331 - protein, 9 aa, synthetic construct
  REGION 1..9: note = Synthetic
ALWYSNLWV

SEQ ID NO: 332 - protein, 9 aa, synthetic construct
  REGION 1..9: note = Synthetic
TGAVTTSNY

SEQ ID NO: 333 - skipped

SEQ ID NO: 334 - protein, 9 aa, synthetic construct
  REGION 1..9: note = Synthetic
ALWYSNLWV

SEQ ID NO: 335 - protein, 9 aa, synthetic construct
  REGION 1..9: note = Synthetic
TGAVTTSNY

SEQ ID NO: 336 - skipped

SEQ ID NO: 337 - protein, 9 aa, synthetic construct
  REGION 1..9: note = Synthetic
ALWYSNLWV

SEQ ID NO: 338 - protein, 9 aa, synthetic construct
  REGION 1..9: note = Synthetic
TGAVTTSNY

SEQ ID NO: 339 - skipped

SEQ ID NO: 340 - protein, 9 aa, synthetic construct
  REGION 1..9: note = Synthetic
ALWYSNLWV

SEQ ID NO: 341 - protein, 9 aa, synthetic construct
  REGION 1..9: note = Synthetic
TGAVTTSNY

SEQ ID NO: 342 - skipped

SEQ ID NO: 343 - protein, 9 aa, synthetic construct
  REGION 1..9: note = Synthetic
ALWYSNLWV

SEQ ID NO: 344 - protein, 9 aa, synthetic construct
  REGION 1..9: note = Synthetic
TGAVTTSNY

SEQ ID NO: 345 - skipped

SEQ ID NO: 346 - protein, 9 aa, synthetic construct
  REGION 1..9: note = Synthetic
ALWYSNLWV

SEQ ID NO: 347 - protein, 9 aa, synthetic construct
  REGION 1..9: note = Synthetic
TGAVTTSNY

SEQ ID NO: 348 - skipped

SEQ ID NO: 349 - protein, 9 aa, synthetic construct
  REGION 1..9: note = Synthetic
ALWYSNLWV

SEQ ID NO: 350 - protein, 9 aa, synthetic construct
  REGION 1..9: note = Synthetic
TGAVTTSNY

SEQ ID NO: 351 - skipped

SEQ ID NO: 352 - protein, 9 aa, synthetic construct
  REGION 1..9: note = Synthetic
ALWYSNLWV

SEQ ID NO: 353 - protein, 9 aa, synthetic construct
  REGION 1..9: note = Synthetic
TGAVTTSNY

SEQ ID NO: 354 - skipped

SEQ ID NO: 355 - protein, 9 aa, synthetic construct
  REGION 1..9: note = Synthetic
ALWYSNLWV

SEQ ID NO: 356 - protein, 9 aa, synthetic construct
  REGION 1..9: note = Synthetic
TGAVTTSNY

SEQ ID NO: 357 - skipped

SEQ ID NO: 358 - protein, 9 aa, synthetic construct
  REGION 1..9: note = Synthetic
ALWYSNLWV

SEQ ID NO: 359 - protein, 9 aa, synthetic construct
  REGION 1..9: note = Synthetic
TGAVTTSNY

SEQ ID NO: 360 - skipped

SEQ ID NO: 361 - protein, 9 aa, synthetic construct
  REGION 1..9: note = Synthetic
ALWYSNLWV

SEQ ID NO: 362 - protein, 9 aa, synthetic construct
  REGION 1..9: note = Synthetic
TGAVTTSNY

SEQ ID NO: 363 - skipped

SEQ ID NO: 364 - protein, 9 aa, synthetic construct
  REGION 1..9: note = Synthetic
ALWYSNLWV

SEQ ID NO: 365 - protein, 9 aa, synthetic construct
  REGION 1..9: note = Synthetic
TGAVTTSNY

SEQ ID NO: 366 - skipped

SEQ ID NO: 367 - protein, 9 aa, synthetic construct
  REGION 1..9: note = Synthetic
ALWYSNLWV

SEQ ID NO: 368 - protein, 9 aa, synthetic construct
  REGION 1..9: note = Synthetic
TGAVTTSNY

SEQ ID NO: 369 - skipped

SEQ ID NO: 370 - protein, 9 aa, synthetic construct
  REGION 1..9: note = Synthetic
ALWYSNLWV

SEQ ID NO: 371 - protein, 9 aa, synthetic construct
  REGION 1..9: note = Synthetic
TGAVTTSNY

SEQ ID NO: 372 - skipped

SEQ ID NO: 373 - protein, 9 aa, synthetic construct
  REGION 1..9: note = Synthetic
ALWYSNLWV

SEQ ID NO: 374 - protein, 9 aa, synthetic construct
  REGION 1..9: note = Synthetic
TGAVTTSNY

SEQ ID NO: 375 - skipped

SEQ ID NO: 376 - protein, 9 aa, synthetic construct
  REGION 1..9: note = Synthetic
ALWYSNLWV

SEQ ID NO: 377 - protein, 9 aa, synthetic construct
  REGION 1..9: note = Synthetic
TGAVTTSNY

SEQ ID NO: 378 - skipped

SEQ ID NO: 379 - protein, 9 aa, synthetic construct
  REGION 1..9: note = Synthetic
ALWYSNLWV

SEQ ID NO: 380 - protein, 9 aa, synthetic construct
  REGION 1..9: note = Synthetic
TGAVTTSNY

SEQ ID NO: 381 - skipped

SEQ ID NO: 382 - protein, 9 aa, synthetic construct
  REGION 1..9: note = Synthetic
ALWYSNLWV

SEQ ID NO: 383 - protein, 9 aa, synthetic construct
  REGION 1..9: note = Synthetic
TGAVTTSNY

SEQ ID NO: 384 - skipped

SEQ ID NO: 385 - protein, 9 aa, synthetic construct
  REGION 1..9: note = Synthetic
ALWYSNLWV

SEQ ID NO: 386 - protein, 9 aa, synthetic construct
  REGION 1..9: note = Synthetic
TGAVTTSNY

SEQ ID NO: 387 - skipped

SEQ ID NO: 388 - protein, 9 aa, synthetic construct
  REGION 1..9: note = Synthetic
ALWYSNLWV

SEQ ID NO: 389 - protein, 9 aa, synthetic construct
  REGION 1..9: note = Synthetic
TGAVTTSNY

SEQ ID NO: 390 - skipped

SEQ ID NO: 391 - protein, 9 aa, synthetic construct
  REGION 1..9: note = Synthetic
ALWYSNLWV

SEQ ID NO: 392 - protein, 9 aa, synthetic construct
  REGION 1..9: note = Synthetic
TGAVTTSNY

SEQ ID NO: 393 - skipped

SEQ ID NO: 394 - protein, 9 aa, synthetic construct
  REGION 1..9: note = Synthetic
ALWYSNLWV

SEQ ID NO: 395 - protein, 9 aa, synthetic construct
  REGION 1..9: note = Synthetic
TGAVTTSNY

SEQ ID NO: 396 - skipped

SEQ ID NO: 397 - protein, 9 aa, synthetic construct
  REGION 1..9: note = Synthetic
ALWYSNLWV

SEQ ID NO: 398 - protein, 9 aa, synthetic construct
  REGION 1..9: note = Synthetic
TGAVTTSNY

SEQ ID NO: 399 - skipped

SEQ ID NO: 400 - protein, 9 aa, synthetic construct
  REGION 1..9: note = Synthetic
ALWYSNLWV

SEQ ID NO: 401 - protein, 9 aa, synthetic construct
  REGION 1..9: note = Synthetic
TGAVTTSNY

SEQ ID NO: 402 - skipped

SEQ ID NO: 403 - protein, 9 aa, synthetic construct
  REGION 1..9: note = Synthetic
ALWYSNLWV

SEQ ID NO: 404 - protein, 9 aa, synthetic construct
  REGION 1..9: note = Synthetic
TGAVTTSNY

SEQ ID NO: 405 - skipped

SEQ ID NO: 406 - protein, 9 aa, synthetic construct
  REGION 1..9: note = Synthetic
ALWYSNLWV

SEQ ID NO: 407 - protein, 9 aa, synthetic construct
  REGION 1..9: note = Synthetic
TGAVTTSNY

SEQ ID NO: 408 - skipped

SEQ ID NO: 409 - protein, 9 aa, synthetic construct
  REGION 1..9: note = Synthetic
ALWYSNLWV

SEQ ID NO: 410 - protein, 9 aa, synthetic construct
  REGION 1..9: note = Synthetic
TGAVTTRNY

SEQ ID NO: 411 - skipped

SEQ ID NO: 412 - protein, 9 aa, synthetic construct
  REGION 1..9: note = Synthetic
ALWYSNLWV

SEQ ID NO: 413 - protein, 9 aa, synthetic construct
  REGION 1..9: note = Synthetic
TGAVTTSTY

SEQ ID NO: 414 - skipped

SEQ ID NO: 415 - protein, 9 aa, synthetic construct
  REGION 1..9: note = Synthetic
ALWYSNLWV

SEQ ID NO: 416 - protein, 9 aa, synthetic construct
  REGION 1..9: note = Synthetic
TGAVSTNYY

SEQ ID NO: 417 - skipped

SEQ ID NO: 418 - protein, 9 aa, synthetic construct
  REGION 1..9: note = Synthetic
ALWYSNLWV

SEQ ID NO: 419 - protein, 9 aa, synthetic construct
  REGION 1..9: note = Synthetic
TGAVPTSNY

SEQ ID NO: 420 - skipped

SEQ ID NO: 421 - protein, 9 aa, synthetic construct
  REGION 1..9: note = Synthetic
ALWYSNLWV

SEQ ID NO: 422 - protein, 9 aa, synthetic construct
  REGION 1..9: note = Synthetic
TGAVPTSLY

SEQ ID NO: 423 - skipped

SEQ ID NO: 424 - protein, 9 aa, synthetic construct
  REGION 1..9: note = Synthetic
ALWYSNLWV

SEQ ID NO: 425 - protein, 9 aa, synthetic construct
  REGION 1..9: note = Synthetic
TGAVTPSNY

SEQ ID NO: 426 - skipped

SEQ ID NO: 427 - protein, 9 aa, synthetic construct
  REGION 1..9: note = Synthetic
ALWYSNLWV

SEQ ID NO: 428 - protein, 9 aa, synthetic construct
  REGION 1..9: note = Synthetic
GGSVTTHNY

SEQ ID NO: 429 - skipped

SEQ ID NO: 430 - protein, 9 aa, synthetic construct
  REGION 1..9: note = Synthetic
ALWYSNLWV

SEQ ID NO: 431 - protein, 9 aa, synthetic construct
  REGION 1..9: note = Synthetic
TGAVTSPSY

SEQ ID NO: 432 - skipped

SEQ ID NO: 433 - protein, 9 aa, synthetic construct
  REGION 1..9: note = Synthetic
ALWYSNLWV

SEQ ID NO: 434 - protein, 9 aa, synthetic construct
  REGION 1..9: note = Synthetic
LGSVTTFNY

SEQ ID NO: 435 - skipped

SEQ ID NO: 436 - protein, 9 aa, synthetic construct
  REGION 1..9: note = Synthetic
ALWYSNLWV

SEQ ID NO: 437 - protein, 9 aa, synthetic construct
  REGION 1..9: note = Synthetic
TGAVTTSNY

SEQ ID NO: 438 - skipped

SEQ ID NO: 439 - protein, 9 aa, synthetic construct
  REGION 1..9: note = Synthetic
ALWYSNLWV

SEQ ID NO: 440 - protein, 9 aa, synthetic construct
  REGION 1..9: note = Synthetic
TGAVTTSNY

SEQ ID NO: 441 - skipped

SEQ ID NO: 442 - protein, 9 aa, synthetic construct
  REGION 1..9: note = Synthetic
ALWYSNLWV

SEQ ID NO: 443 - protein, 9 aa, synthetic construct
  REGION 1..9: note = Synthetic
TGAVTTSNY

SEQ ID NO: 444 - skipped

SEQ ID NO: 445 - protein, 9 aa, synthetic construct
  REGION 1..9: note = Synthetic
ALWYSNLWV

SEQ ID NO: 446 - protein, 9 aa, synthetic construct
  REGION 1..9: note = Synthetic
TGAVTTSNY

SEQ ID NO: 447 - skipped

SEQ ID NO: 448 - protein, 9 aa, synthetic construct
  REGION 1..9: note = Synthetic
ALWYSNLWV

SEQ ID NO: 449 - protein, 9 aa, synthetic construct
  REGION 1..9: note = Synthetic
TGAVTTSNY

SEQ ID NO: 450 - skipped

SEQ ID NO: 451 - protein, 9 aa, synthetic construct
  REGION 1..9: note = Synthetic
ALYYSFSWV

SEQ ID NO: 452 - protein, 9 aa, synthetic construct
  REGION 1..9: note = Synthetic
TGAVTTSNY

SEQ ID NO: 453 - skipped

SEQ ID NO: 454 - protein, 9 aa, synthetic construct
  REGION 1..9: note = Synthetic
QTWGNNIWV

SEQ ID NO: 455 - protein, 9 aa, synthetic construct
  REGION 1..9: note = Synthetic
TGAVTTSNY

SEQ ID NO: 456 - skipped

SEQ ID NO: 457 - protein, 9 aa, synthetic construct
  REGION 1..9: note = Synthetic
AIWYSRVWV

SEQ ID NO: 458 - protein, 9 aa, synthetic construct
  REGION 1..9: note = Synthetic
TGAVTTSNY

SEQ ID NO: 459 - skipped

SEQ ID NO: 460 - protein, 9 aa, synthetic construct
  REGION 1..9: note = Synthetic
AIWYSTSWV

SEQ ID NO: 461 - protein, 9 aa, synthetic construct
  REGION 1..9: note = Synthetic
TGAVTTSNY

SEQ ID NO: 462 - skipped

SEQ ID NO: 463 - protein, 9 aa, synthetic construct
  REGION 1..9: note = Synthetic
VIWHSGAWV

SEQ ID NO: 464 - protein, 9 aa, synthetic construct
  REGION 1..9: note = Synthetic
TGAVTTSNY

SEQ ID NO: 465 - skipped

SEQ ID NO: 466 - protein, 9 aa, synthetic construct
  REGION 1..9: note = Synthetic
VVWHSSLWL

SEQ ID NO: 467 - protein, 9 aa, synthetic construct
  REGION 1..9: note = Synthetic
TGAVTTSNY

SEQ ID NO: 468 - skipped

SEQ ID NO: 469 - protein, 9 aa, synthetic construct
  REGION 1..9: note = Synthetic
LLSYSRSWV

SEQ ID NO: 470 - protein, 9 aa, synthetic construct
  REGION 1..9: note = Synthetic
TGAVTTSNY

SEQ ID NO: 471 - skipped

SEQ ID NO: 472 - protein, 9 aa, synthetic construct
  REGION 1..9: note = Synthetic
LLFYNRLWV

SEQ ID NO: 473 - protein, 9 aa, synthetic construct
  REGION 1..9: note = Synthetic
TGAVTTSNY

SEQ ID NO: 474 - skipped

SEQ ID NO: 475 - protein, 9 aa, synthetic construct
  REGION 1..9: note = Synthetic
VIWHGSVWV

SEQ ID NO: 476 - protein, 9 aa, synthetic construct
  REGION 1..9: note = Synthetic
TGAVTTSNY

SEQ ID NO: 477 - skipped

SEQ ID NO: 478 - protein, 9 aa, synthetic construct
  REGION 1..9: note = Synthetic
ANWKSNILV

SEQ ID NO: 479 - protein, 9 aa, synthetic construct
  REGION 1..9: note = Synthetic
TGAVTTSNY

SEQ ID NO: 480 - skipped

SEQ ID NO: 481 - protein, 9 aa, synthetic construct
  REGION 1..9: note = Synthetic
RIWHSSSWV

SEQ ID NO: 482 - protein, 9 aa, synthetic construct
  REGION 1..9: note = Synthetic
TGAVTTSNY

SEQ ID NO: 483 - skipped

SEQ ID NO: 484 - protein, 9 aa, synthetic construct
  REGION 1..9: note = Synthetic
RLDYSGVWV

SEQ ID NO: 485 - protein, 9 aa, synthetic construct
  REGION 1..9: note = Synthetic
TGAVTTSNY

SEQ ID NO: 486 - skipped

SEQ ID NO: 487 - protein, 9 aa, synthetic construct
  REGION 1..9: note = Synthetic
VIWHNLAWV

SEQ ID NO: 488 - protein, 9 aa, synthetic construct
  REGION 1..9: note = Synthetic
TGAVTTSNY

SEQ ID NO: 489 - skipped

SEQ ID NO: 490 - protein, 9 aa, synthetic construct
  REGION 1..9: note = Synthetic
TIWHSGVWV

SEQ ID NO: 491 - protein, 9 aa, synthetic construct
  REGION 1..9: note = Synthetic
TGAVTTSNY

SEQ ID NO: 492 - skipped

SEQ ID NO: 493 - protein, 9 aa, synthetic construct
  REGION 1..9: note = Synthetic
AIWPGDAWV

SEQ ID NO: 494 - protein, 9 aa, synthetic construct
  REGION 1..9: note = Synthetic
TGAVTTSNY

SEQ ID NO: 495 - skipped

SEQ ID NO: 496 - protein, 9 aa, synthetic construct
  REGION 1..9: note = Synthetic
LTWHTSAWV

SEQ ID NO: 497 - protein, 9 aa, synthetic construct
  REGION 1..9: note = Synthetic
TGAVTTSNY

SEQ ID NO: 498 - skipped

SEQ ID NO: 499 - protein, 9 aa, synthetic construct
  REGION 1..9: note = Synthetic
QAWYSNIGV

SEQ ID NO: 500 - protein, 9 aa, synthetic construct
  REGION 1..9: note = Synthetic
TGAVTTSNY

SEQ ID NO: 501 - skipped

SEQ ID NO: 502 - protein, 9 aa, synthetic construct
  REGION 1..9: note = Synthetic
AIWYSTAYI

SEQ ID NO: 503 - protein, 9 aa, synthetic construct
  REGION 1..9: note = Synthetic
TGAVTTSNY

SEQ ID NO: 504 - skipped

SEQ ID NO: 505 - protein, 9 aa, synthetic construct
  REGION 1..9: note = Synthetic
LIWYNNIWV

SEQ ID NO: 506 - protein, 9 aa, synthetic construct
  REGION 1..9: note = Synthetic
TGAVTTSNY

SEQ ID NO: 507 - skipped

SEQ ID NO: 508 - protein, 9 aa, synthetic construct
  REGION 1..9: note = Synthetic
RVWHSDAWV

SEQ ID NO: 509 - protein, 9 aa, synthetic construct
  REGION 1..9: note = Synthetic
TGAVTTSNY

SEQ ID NO: 510 - skipped

SEQ ID NO: 511 - protein, 9 aa, synthetic construct
  REGION 1..9: note = Synthetic
LIWYGSAWV

SEQ ID NO: 512 - protein, 9 aa, synthetic construct
  REGION 1..9: note = Synthetic
TGAVTTSNY

SEQ ID NO: 513 - skipped

SEQ ID NO: 514 - protein, 9 aa, synthetic construct
  REGION 1..9: note = Synthetic
AAWDVSLWV

SEQ ID NO: 515 - protein, 9 aa, synthetic construct
  REGION 1..9: note = Synthetic
TGAVTTSNY

SEQ ID NO: 516 - skipped

SEQ ID NO: 517 - protein, 9 aa, synthetic construct
  REGION 1..9: note = Synthetic
AAWHDNAWV

SEQ ID NO: 518 - protein, 9 aa, synthetic construct
  REGION 1..9: note = Synthetic
TGAVTTSNY

SEQ ID NO: 519 - skipped

SEQ ID NO: 520 - protein, 9 aa, synthetic construct
  REGION 1..9: note = Synthetic
QPYYSNNWV

SEQ ID NO: 521 - protein, 9 aa, synthetic construct
  REGION 1..9: note = Synthetic
TGAVTTSNY

SEQ ID NO: 522 - skipped

SEQ ID NO: 523 - protein, 9 aa, synthetic construct
  REGION 1..9: note = Synthetic
QAWDRSIWV

SEQ ID NO: 524 - protein, 9 aa, synthetic construct
  REGION 1..9: note = Synthetic
TGAVTTSNY

SEQ ID NO: 525 - skipped

SEQ ID NO: 526 - protein, 9 aa, synthetic construct
  REGION 1..9: note = Synthetic
LLYYGSIWV

SEQ ID NO: 527 - protein, 9 aa, synthetic construct
  REGION 1..9: note = Synthetic
TGAVTTSNY

SEQ ID NO: 528 - skipped

SEQ ID NO: 529 - protein, 9 aa, synthetic construct
  REGION 1..9: note = Synthetic
AIWYDNIWV

SEQ ID NO: 530 - protein, 9 aa, synthetic construct
  REGION 1..9: note = Synthetic
TGAVTTSNY

SEQ ID NO: 531 - skipped

SEQ ID NO: 532 - protein, 9 aa, synthetic construct
  REGION 1..9: note = Synthetic
LSWHSSSWV

SEQ ID NO: 533 - protein, 9 aa, synthetic construct
  REGION 1..9: note = Synthetic
TGAVTTSNY

SEQ ID NO: 534 - skipped

SEQ ID NO: 535 - protein, 9 aa, synthetic construct
  REGION 1..9: note = Synthetic
LIWQTSAWV

SEQ ID NO: 536 - protein, 9 aa, synthetic construct
  REGION 1..9: note = Synthetic
TGAVTTSNY

SEQ ID NO: 537 - skipped

SEQ ID NO: 538 - protein, 9 aa, synthetic construct
  REGION 1..9: note = Synthetic
AAWHDNAWV